(19) United States
(12) Patent Application Publication
(10) Pub. No.: US 2024/0246981 A1
(43) Pub. Date: Jul. 25, 2024

(54) PHENYLPIPERIDINE DERIVATIVES AS INHIBITORS OF GLUTAMINYL-PEPTIDE CYCLOTRANSFERASE AND GLUTAMINYL-PEPTIDE CYCLOTRANSFERASE LIKE PROTEIN

(71) Applicant: Boehringer Ingelheim International GmbH, Ingelheim am Rhein (DE)

(72) Inventors: Jens WILLWACHER, Biberach an der Riss (DE); Florian Paul Christian BINDER, Maselheim (DE); Georg DAHMANN, Biberach (DE); Sandra Ruth HANDSCHUH, Biberach an der Riss (DE); Sophia Astrid REINDL, Mittelbiberach (DE); James Young Soo YANG HAMILTON, Biberach (DE)

(21) Appl. No.: 18/542,910

(22) Filed: Dec. 18, 2023

(30) Foreign Application Priority Data
Dec. 22, 2022 (EP) 22216126.7

(51) Int. Cl. C07D 487/04; C07D 401/14; C07D 407/14; C07D 413/14; C07D 417/14; C07D 471/04; C07D 491/044; C07D 491/056; C07D 498/04; C07D 513/04

(52) U.S. Cl. CPC C07D 487/04; C07D 401/14; C07D 407/14; C07D 413/14; C07D 417/14; C07D 471/04; C07D 491/044; C07D 491/056; C07D 498/04; C07D 513/04

(57) ABSTRACT

The present disclosure provides certain phenylpiperidine derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### TECHNICAL FIELD

The present disclosure provides certain phenylpiperidine derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### BACKGROUND INFORMATION

Glutaminyl-peptide cyclotransferase (QPCT) and glutaminyl-peptide cyclotransferase-like protein (QPCTL) catalyze the intramolecular cyclization of N-terminal glutamine (Q) residues into pyroglutamic acid (pE) liberating ammonia [Stephan Schilling et al., “Identification of Human Glutaminyl Cyclase as a Metalloenzyme POTENT INHIBITION BY IMIDAZOLE DERIVATIVES AND HETEROCYCLIC CHELATORS,” Journal of Biological Chemistry 278, no. 50 (2003): 49773-79, https://doi.org/10.1074/jbc.m309077200; Holger Cynis et al., “Isolation of an Isoenzyme of Human Glutaminyl Cyclase: Retention in the Golgi Complex Suggests Involvement in the Protein Maturation Machinery,” Journal of Molecular Biology 379, no. 5 (2008): 966-80, https://doi.org/10.1016/j.jmb.2008.03.078; Anett Stephan et al., “Mammalian Glutaminyl Cyclases and Their Isoenzymes Have Identical Enzymatic Characteristics,” FEBS Journal 276, no. 22 (2009): 6522-36, https://doi.org/10.1111/j.1742-4658.2009.07337.x.]. While QPCT is a secreted protein, QPCTL is retained within the Golgi complex. Both enzymes share a high homology in the active site and similar catalytic specificity. Because of the high homology in the active site, inhibition of the active site blocks the enzymatic activity of both enzymes: QPCT and QPCTL. Hence the term “QPCT/L” describes both enzymes at once. Due to their different cellular localisation, differences in their relevance for modification of biological substrates have been reported. Known substrates of the intracellular QPCTL and/or extracellular QPCT are CD47 [Meike E. W. Logtenberg et al., “Glutaminyl Cyclase Is an Enzymatic Modifier of the CD47-SIRPα Axis and a Target for Cancer Immunotherapy,” Nature Medicine 25, no. 4 (2019): 612-19, https://doi.org/10.1038/s41591-019-0356-z.], different chemokines (like for example CCL2 and 7 or CX3CL1) [Rosa Barreira da Silva et al., “Loss of the Intracellular Enzyme QPCTL Limits Chemokine Function and Reshapes Myeloid Infiltration to Augment Tumor Immunity,” Nature Immunology 23, no. 4 (2022): 568-80, https://doi.org/10.1038/s41590-022-01153-x; Astrid Kehlen et al., “N-Terminal Pyroglutas mate Formation in CX3CL1 Is Essential for Its Full Biologic Activity,” Bioscience Reports 37, no. 4 (2017): BSR20170712, https://doi.org/10.1042/bsr20l70712.], Amyloid-b peptides [Cynis et al., “Isolation of an Isoenzyme of Human Glutaminyl Cyclase: Retention in the Golgi Complex Suggests Involvement in the Protein Maturation Machinery.” ] or hormones like TRH [Andreas Becker et al., “IsoQC (QPCTL) Knock-out Mice Suggest Differential Substrate Conversion by Glutaminyl Cyclase Isoenzymes,” Biological Chemistry 397, no. 1 (2016): 45-55, https://doi.org/10.1515/hsz-2015-0192.]. The modification of N-terminal glutamine to pyroglutamate on the substrates has functional consequences for the proteins and could impact different pathomechanisms in several diseases. CD47 is expressed on the cell surface of virtually all cells of the body, including apoptotic cells, senescent cells or cancer cells. [Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011]. The main ligand for CD47 is signal-regulatory protein alpha (SIRPα), an inhibitory transmembrane receptor present on myeloid cells, such as macrophages, monocytes, neutrophils, dendritic cells and others. QPCTL mediated N-terminal pyroglutamate modification on CD47 is required for SIRPα binding [Deborah Hatherley et al., “Paired Receptor Specificity Explained by Structures of Signal Regulatory Proteins Alone and Complexed with CD47,” Molecular Cell 31, no. 2 (2008): 266-77, https://doi.org/10.1016/j.molcel.2008.05.026; Meike E. W. Logtenberg et al., “Glutaminyl Cyclase Is an Enzymatic Modifier of the CD47-SIRPα Axis and a Target for Cancer Immunotherapy,” Nature Medicine 25, no. 4 (2019): 612-19, https://doi.org/10.1038/s41591-019-0356-z.] This signaling axis induces a “Don't Eat Me Signal”, preventing engulfment of CD47 expressing cells by macrophages. Thus, high expression of CD47 is connected to the pathogenesis of cancer [Logtenberg et al., “Glutaminyl Cyclase Is an Enzymatic Modifier of the CD47-SIRPα Axis and a Target for Cancer Immunotherapy,” 2019; Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011.], COVID-19 [Katie-May McLaughlin et al., “A Potential Role of the CD47/SIRPalpha Axis in COVID-19 Pathogenesis,” Current Issues in Molecular Biology 43, no. 3 (2021): 1212-25, https://doi.org/10.3390/cimb43030086.], lung fibrosis [Gerlinde Wernig et al., “Unifying Mechanism for Different Fibrotic Diseases,” Proceedings of the National Academy of Sciences 114, no. 18 (2017): 4757-62, https://doi.org/10.1073/pnas.1621375114; Lu Cui et al., “Activation of JUN in Fibroblasts Promotes Pro-Fibrotic Programme and Modulates Pros tective Immunity,” Nature Communications 11, no. 1 (2020): 2795, https://doi.org/10.1038/s41467-020-16466-4.], systemic sclerosis [Wernig et al., “Unifying Mechanism for Different Fibrotic Diseases”; Tristan Lerbs et al., “CD47 Prevents the Elimination of Diseased Fibroblasts in Scleroderma,” JCI Insight 5, no. 16 (2020): e140458, https://doi.org/10.1172/jci.insight.140458.] and liver fibrosis [Taesik Gwag et al., “AntiCD47 Antibody Treatment Attenuates Liver Inflammation and Fibrosis in Experimental Non-alcoholic Steatohepatitis Models,” Liver International 42, no. 4 (2022): 829-41, https://doi.org/10.1111/liv.15182.]. Since enhanced CD47 expression blocks the clearance of apoptotic cells, there is an accrual of apoptotic lung epithelial cells, leading to a profibrotic stimulus and accelerating lung inflammation and -scaring [Alexandra L. McCubbrey and Jeffrey L. Curtis, “Efferocytosis and Lung Disease,” Chest 143, no. 6 (2013): 1750-57, https://doi.org/10.1378/chest.12-2413; Brennan D. Gerlach et al., “Efferocytosis Induces Macrophage Proliferation to Help Resolve Tissue Injury,” Cell Metabolism, 2021, https://doi.org/10.1016/j.cmet.2021.10.015.]. Since CD47 half-life and function is majorly dependent on QPCTL enzyme activity, QPCT and QPCTL inhibition could be a suitable mechanism as a treatment in lung fibrosis such as IPF or SSC-ILD [Lerbs et al., “CD47 Prevents the Elimination of Diseased Fibroblasts in Scleroderma.” ], alone or together with current standard of care in pulmonary fibrosis like Nintedanib [Luca Richeldi et al., “Efficacy and Safety of Nintedanib in Idiopathic Pulmonary Fibrosis,” The New England Journal of Medicine 370, no. 22 (2014): 2071-82, https://doi.org/10.1056/nejmoa1402584; Kevin R Flaherty et al., “Nintedanib in Progressive Fibrosing Interstitial Lung Diseases,” New England Journal of Medicine 381, no. 18 (2019): 1718-27, https://doi.org/10.1056/nejmoal9O8681.] or future treatments like a PDE4 inhibitor [Luca Richeldi et al., “Trial of a Preferential Phosphodiesterase 4B Inhibitor for Idiopathic Pulmonary Fibrosis,” New England Journal of Medicine 386, no. 23 (2022): 2178-87, https://doi.org/10.1056/nejmoa2201737].

By expression of CD47, cancer cells can evade destruction by the immune system or evade immune surveillance, e.g. by evading phagocytosis by immune cells [Stephen B. Willingham et al., “The CD47-Signal Regulatory Protein Alpha (SIRPa) Interaction Is a Therapeutic Target for Human Solid Tumors,” Proceedings of the National Academy of Sciences 109, no. 17 (2012): 6662-67, https://doi.org/10.1073/pnas.1121623109].

In addition to CD47, chemokines, such as CCL2 and CX3CL1, have been identified as QPCTL and/or QPCT substrates [Holger Cynis et al., “The Isoenzyme of Glutaminyl Cyclase Is an Important Regulator of Monocyte Infiltration under Inflammatory Conditions,” EMBO Molecular Medicine 3, no. 9 (2011): 545-58, https://doi.org/10.1002/emmm.201100158]. The formation of the N-terminal pGlu was shown to increase in vivo activity, both by conferring resistance to aminopeptidases and by increasing its capacity to induce chemokine receptor signaling. Two main monocyte chemoattractants CCL2 and CCL7 are insensitive to DPP4-inactivation in vivo because of an intracellular mechanism of N-terminal cyclization mediated by the Golgi-associated enzyme QPCTL. It has been shown that QPCTL is a critical regulator of monocyte migration into solid tumors [Kaspar Bresser et al., “QPCTL Regulates Macrophage and Monocyte Abundance and Inflammatory Signatures in the Tumor Microenvironment,” Oncoimmunology 11, no. 1 (2022): 2049486, https://doi.org/10.1080/2162402x.2022.2049486; Rosa Barreira da Silva et al., “Loss of the Intracellular Enzyme QPCTL Limits Chemokine Function and Reshapes Myeloid Infiltration to Augment Tumor Immunity,” Nature Immunology, 2022, 1-13, https://doi.org/10.1038/s41590-022-01153-x]. Targeting of chemokines has long been pursued as a potential strategy for modulating cellular trafficking in different disease settings.

It is therefore desirable to provide potent QPCT/L inhibitors.

Jimenez-Sanchez, et al., Nature Chemical Biology, 2015, 11, 347-357, (hereinafter “J-S, NCB 2015”) discloses the human glutaminyl cyclase (hQC) inhibitors SEN177 and SEN180:

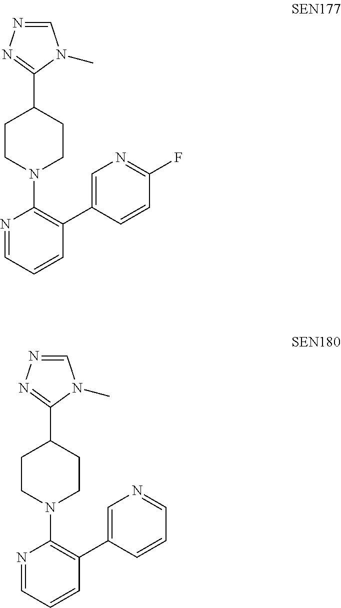 [SMILES: Cn1cnnc1C1CCN(c2ncccc2-c2ccc(F)nc2)CC1 ; Cn1cnnc1C1CCN(c2ncccc2-c2cccnc2)CC1]

SEN177 is disclosed therein (supplementary information) as having a IC₅₀ on isolated hQC of 53 nM and on isolated QPCTL of 13 nM. SEN180 is disclosed therein (supplementary information) as having a IC₅₀ on hQC of 170 nM and on QPCTL of 58 nM.

Pozzi, C, et al, Journal of Biological Inorganic Chemistry, 2018, 23, (8), 1219-1226, (hereinafter “P, JBIC 2018”), further discloses SEN177 and its binding mode within the hQC cavity. SEN177 is disclosed therein as having a Kᵢ on isolated hQC of 20 nM.

WO 2018/178384 discloses QPCTL inhibitors of the general formula A-B-D-E, which include examples 1094 and 1095 (Formula (XIIa) on page 123 and table on page 125):

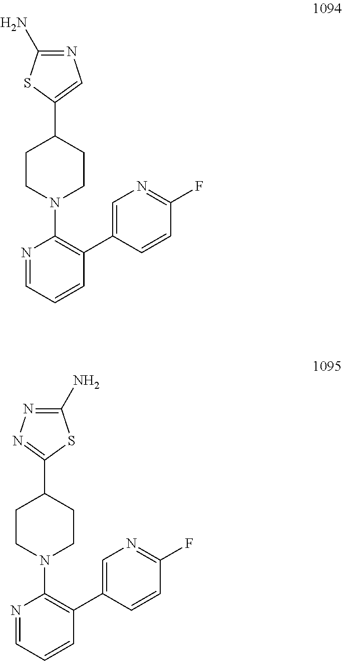

WO 2018/178384 does not disclose any biological data for examples 1094 or 1095.

WO 2022/086920 discloses QPCTL inhibitors of the general formula

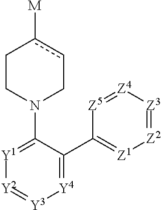

which include compounds 3 and 6:

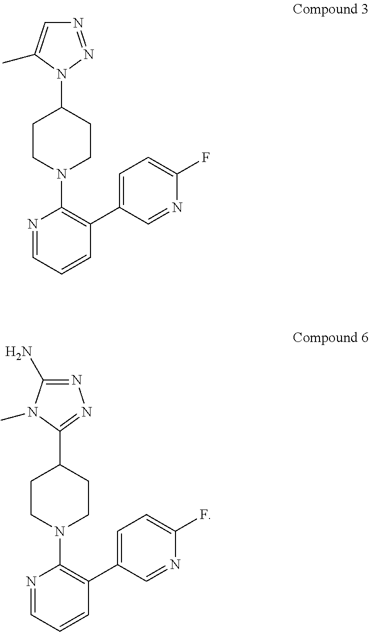 [SMILES: Cc1cnnn1C1CCN(c2ncccc2-c2ccc(F)nc2)CC1 ; Cn1c(N)nnc1C1CCN(c2ncccc2-c2ccc(F)nc2)CC1]

The chemical name of Compound 3 is disclosed in WO 2022/086920 as “1-(1-(6′-chloro[3,3′-bipyridin]-2-yl)piperidin-4-yl)-1H-1,2,3-triazol-4-amine” which does not correspond to the chemical structure disclosed therein, but to an alternative structure in which the fluorine atom is replaced with chlorine:

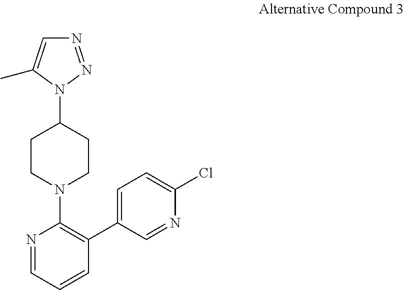 [SMILES: Cc1cnnn1C1CCN(c2ncccc2-c2ccc(Cl)nc2)CC1]

Compounds 3 (including alternative compound 3) and 6 in WO 2022/086920 are disclosed therein [00343] as having inhibitory activity on isolated QPCTL of IC₅₀<1 μM.

CN 114874186 discloses glutamine acyl cyclase isoenzyme inhibitors of the general formula

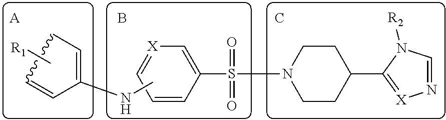

which includes examples 21 and 23 (table on page 17):

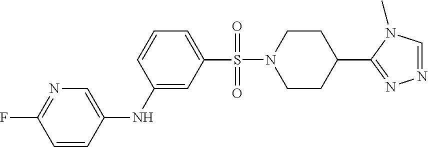 [SMILES: Cn1cnnc1C1CCN(S(=O)(=O)c2cccc(Nc3ccc(F)nc3)c2)CC1]

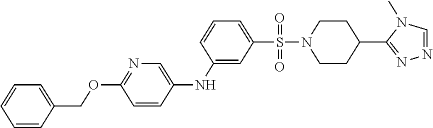 [SMILES: Cn1cnnc1C1CCN(S(=O)(=O)c2cccc(Nc3ccc(OCc4ccccc4)nc3)c2)CC1]

IC₅₀'s are given in CN 114874186 for examples 21 and 23 as 29.22 nM and 11.26 nM respectively.

### DETAILED DESCRIPTION OF THE INVENTION

The present invention discloses novel phenylpiperidine derivatives of formula (I)

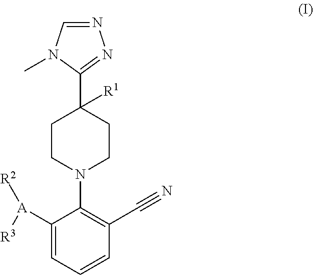

that are inhibitors of Glutaminyl-peptide cyclotransferase (QPCT) and glutaminyl-peptide cyclotransferase-like protein (QPCTL), possessing appropriate pharmacological and pharmacokinetic properties enabling their use as medicaments for the treatment of conditions and/or diseases treatable by inhibition of QPCT/L.

The compounds of the present invention may provide several advantages, such as enhanced potency, cellular potency, high metabolic and/or chemical stability, high selectivity, safety and tolerability, enhanced solubility, enhanced permeability, desirable plasma protein binding, enhanced bioavailability, suitable pharmacokinetic profiles, and the possibility to form stable salts.

### Compounds of the Invention

The present invention provides novel phenylpiperidine derivatives that surprisingly, are potent inhibitors of QPCT and QPCTL (Assay A), as well as potent inhibitors of QPCT/L in cells relevant for, but not limited to, lung diseases or cancer (Assay B).

Furthermore, the present novel phenylpiperidine derivatives have appropriate membrane permeability and a low in vitro efflux (Assay C).

Consequently, compounds of the present invention are more viable for human use.

Compounds of the present invention differ structurally from SEN177 and SEN180 in J-S, NCB 2015, in that the phenyl ring instead of a pyridyl ring is attached to the piperidinyl ring.

Furthermore, a carbonitrile substituent is attached at the ortho-position to the piperidinyl ring attachment position of said phenyl ring. Still further, R¹ is not limited to hydrogen, and A represents substituted heterocyclic ring systems beyond pyridinyl.

Compounds of the present invention differ structurally from examples 1094 and 1095 in WO 2018/178384 in that a phenyl ring instead of a pyridyl ring is attached to the piperidinyl ring. Furthermore, a carbonitrile substituent is attached at the ortho-position to the piperidinyl ring attachment position of said phenyl ring. Still further, R¹ is not limited to hydrogen and A represents heterocyclic ring systems beyond pyridinyl. Still further, the 5-membered heterocyclic ring attached to the piperidinyl ring at the 4-position relative to the piperidinyl nitrogen is in example 1094 an aminothiazolyl ring and in example 1095 an aminothiadiazolyl ring whereas in compounds of the present invention it is a 3-substituted-4-methyl-4H-1,2,4-triazolyl ring.

Compounds of the present invention differ structurally from compounds 3 (including alters native compound 3) and 6 in WO 2022/086920 in that a phenyl ring instead of a pyridinyl ring, is attached to the piperidinyl ring. Furthermore, a carbonitrile substituent is attached at the ortho-position to the piperidinyl ring attachment position of said phenyl ring. Still further, R¹ is not limited to hydrogen, and A represents heterocyclic ring systems beyond pyridinyl. Still further, the 5-membered heterocyclic ring “M” in the general formula of WO 2022/086920 is in compound 3 a regioisomer of the 3-substituted 4-methyl-4H-1,2,4-triazolyl ring in compounds of the present invention, and the 5-membered heterocyclic ring “M” in the general formula of WO 2022/086920 in compound 4 is a 3-substituted 4-methyl-4H-1,2,4-triazolyl ring as in compounds of the present invention but that it bears an amino group.

Compounds of the present invention differ structurally from compounds 21 and 23 in CN114874186 in that the central sulfonamide moiety linking the piperidinyl ring to the phenyl ring is replaced by a direct bond. Furthermore, a carbonitrile substituent is attached at the ortho-position to the piperidinyl ring attachment position of said phenyl ring. Still further, compounds of the present invention do not contain an amino linker between said phenyl ring and a further cyclic ring.

These structural differences between compounds of the present invention and the prior art unexpectedly lead to a favourable combination of (i) potent inhibition of QPCT and QPCTL, (ii) potent inhibition of QPCT/L in cells relevant for, but not limited to, lung diseases or cancer, and (iii) appropriate membrane permeability and a low in vitro efflux.

Compounds of the invention are thus superior to those disclosed in the prior art in terms of the combination of the following parameters:
potent inhibition of QPCT and QPCTL (Assay A)
potent inhibition of QPCT/L in cells relevant for, but not limited to, lung diseases or cancer (Assay B)
appropriate membrane permeability and a low in vitro efflux (Assay C)

The present invention provides novel compounds according to formula (I)

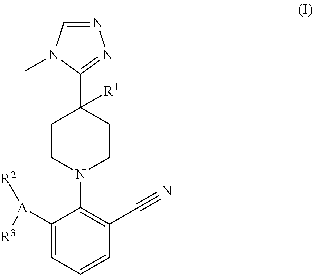

wherein
A is A1a which is a 5- or 6-membered mono-heteroaryl ring containing one or two heteroatom members selected from the group consisting of nitrogen, oxygen and sulphur;
or A is A1b which is a 9- or 10-membered fused bicyclic-heteroaryl ring containing one to four heteroatom members selected from the group consisting of nitrogen, oxygen and sulphur, wherein at least one of the heteroatom members is nitrogen;
or A is selected from the group A1c consisting of

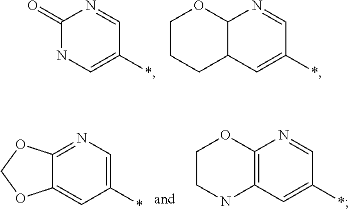

R¹ is selected from the group R1a, consisting of H, C₁₋₄-alkyl and halo;
R² is selected from the group R2a, consisting of H, halo, hydroxy, C₁₋₆-alkyl, C₂₋₆-alkynyl, C₃₋₆-cycloalkyl, F₁₋₉-fluoro-C₁₋₄-alkyl, HO—C₁₋₆-alkyl, C₁₋₆-alkyloxy, C₁₋₄-alkyl-O—H₂CH₂C—O—, C₃₋₆-cycloalkyloxy, C₃₋₆-cycloalkyl-H₂C—O—, F₁₋₉-fluoro-C₁₋₄-alkyloxy, C₁₋₆-alkyl-O—C(O)—, H₂N—C(O)—, and C₁₋₆-alkyl-NH—C(O)—;
or R² is selected from the group R2b, consisting of phenyl, benzyl, phenoxy and benzyloxy, wherein R2b is substituted with one or two R⁴;
or R² is R2c, which is a 5- or 6-membered mono-heteroaryl ring containing one or two heteroatom members selected from the group consisting of nitrogen, oxygen and sulphur, wherein R2c is substituted with one or two R⁴;
or R² is R2d, consisting of

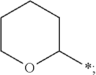

R³ is selected from the group R3a, consisting of H, C₁₋₄-alkyl and halo;
R⁴ is selected from the group R4a, consisting of H, C₁₋₄-alkyl and halo; or a salt thereof, particularly a pharmaceutically acceptable salt thereof.

Another embodiment of the present invention relates to a compound of formula (I), wherein A is A2 which is a 5- or 6-membered mono-heteroaryl ring containing one or two heteroatom members selected from the group consisting of nitrogen and oxygen; and substituents R¹, R², R³ and R⁴ are defined as in any of the preceding embodiments.

Another embodiment of the present invention relates to a compound of formula (I), wherein A is A3 which is a 9- or 10-membered fused bicyclic-heteroaryl ring containing one to four heteroatom members selected from the group consisting of nitrogen and oxygen, wherein at least one of the heteroatom members is nitrogen; and substituents R¹, R², R³ and R⁴ are defined as in any of the preceding embodiments.

Another embodiment of the present invention relates to a compound of formula (I), wherein A is selected from the group A4 consisting of pyrazolyl, pyridinyl, pyrimidinyl, pyridazinyl, pyrazinyl, methyl-pyrimidonyl, thiazolyl, isothiazolyl, oxazolyl, isoxazolyl, 1,2-dihydropyrimidin-2-onyl, 3H-imidazo[4,5-b]pyridinyl, imidazo[1,2-a]pyrimidinyl, 2H-pyrazolo[3,4-b]pyridinyl, 1H-[1,2,3]triazolo[4,5-b]pyridinyl, [1,2,4]triazolo[4,3-a]pyrimidinyl, 1H-pyrazolo[4,3-c]pyridinyl, [1,2,5]oxadiazolo[3,4-b]pyridinyl, [1,2,4]triazolo[1,5-a]pyrimidinyl, [1,2,5]thiadiazolo[3,4-b]pyridinyl, 2H-[1,3]dioxolo[4,5-b]pyridinyl, imidazo[1,2-a]pyrimidinyl, pyrazolo[1,5-b]pyridazinyl, 2H,3H,4H-pyrano[2,3-b]pyridinyl, 1H,2H,3H-pyrido[2,3-b][1,4]oxazinyl and 1,8-naphthyridinyl;
and substituents R¹, R², R³ and R⁴ are defined as in any of the preceding embodiments.

Another embodiment of the present invention relates to a compound of formula (I), wherein A is selected from the group A5 consisting of pyridinyl, pyrimidinyl, pyridazinyl, pyrazinyl, pyrimidonyl and isothiazolyl;
and substituents R¹, R², R³ and R⁴ are defined as in any of the preceding embodiments.

Another embodiment of the present invention relates to a compound of formula (I), wherein A is selected from the group A6 consisting of pyridinyl, pyridazinyl, 2H-pyrazolo[3,4-b]pyridinyl and pyrazolo[1,5-b]pyridazinyl;
and substituents R¹, R², R³ and R⁴ are defined as in any of the preceding embodiments.

Another embodiment of the present invention relates to a compound of formula (I), wherein A is selected from the group A7 consisting of

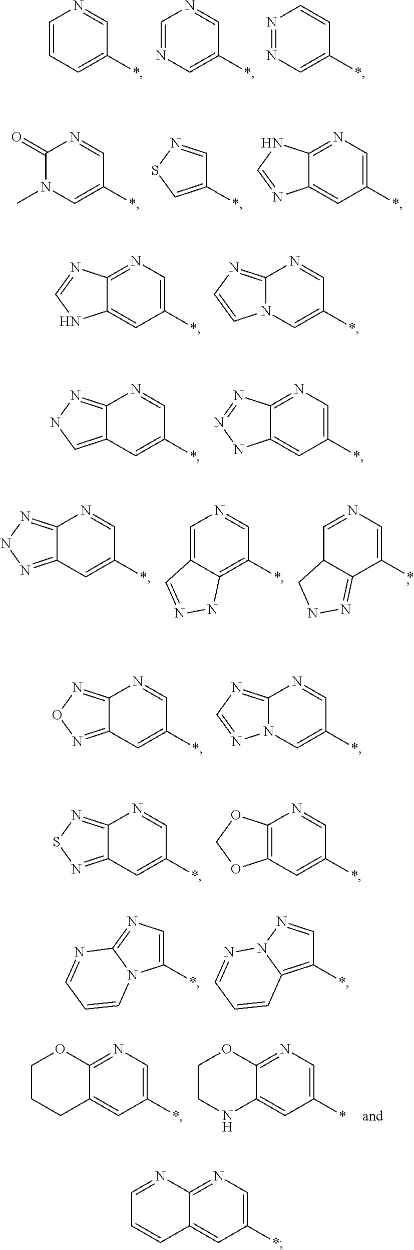

and substituents R¹, R², R³ and R⁴ are defined as in any of the preceding embodiments.

Another embodiment of the present invention relates to a compound of formula (I), wherein A is selected from the group A8 consisting of

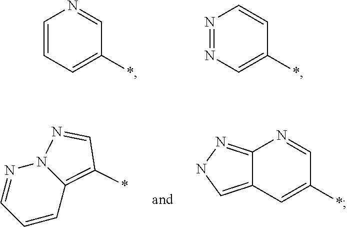

and substituents R¹, R², R³ and R⁴ are defined as in any of the preceding embodiments.

Another embodiment of the present invention relates to a compound of formula (I), wherein A is selected from the group A9 consisting of

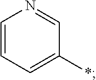

and substituents R¹, R², R³ and R⁴ are defined as in any of the preceding embodiments.

Another embodiment of the present invention relates to a compound of formula (I), wherein A is selected from the group A10 consisting of

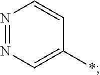

Another embodiment of the present invention relates to a compound of formula (I), wherein A is selected from the group A11 consisting of

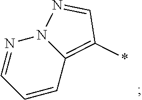 [SMILES: *c1cnn2ncccc12]

and substituents R¹, R², R³ and R⁴ are defined as in any of the preceding embodiments.

Another embodiment of the present invention relates to a compound of formula (I), wherein A is selected from the group A12 consisting of

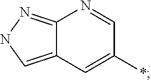

and substituents R¹, R², R³ and R⁴ are defined as in any of the preceding embodiments.

Another embodiment of the present invention relates to a compound of formula (I), wherein R¹ is selected from the group R1b, consisting of H, H₃C—, H₃CH₂C—, H₃CH₂CH₂C—, (H₃C)₂HC—, Cl and F;
and substituents A, R², R³ and R⁴ are defined as in any of the preceding embodiments.

Another embodiment of the present invention relates to a compound of formula (I), wherein R¹ is selected from the group R1c, consisting of H, H₃C—, Cl and F;
and substituents A, R², R³ and R⁴ are defined as in any of the preceding embodiments.

Another embodiment of the present invention relates to a compound of formula (I), wherein R¹ is selected from the group R1d, consisting of H, H₃C— and F;
and substituents A, R², R³ and R⁴ are defined as in any of the preceding embodiments.

Another embodiment of the present invention relates to a compound of formula (I), wherein R¹ is selected from the group R1e, consisting of H;
and substituents A, R², R³ and R⁴ are defined as in any of the preceding embodiments.

Another embodiment of the present invention relates to a compound of formula (I), wherein R¹ is selected from the group R1f, consisting of H₃C—;
and substituents A, R², R³ and R⁴ are defined as in any of the preceding embodiments.

Another embodiment of the present invention relates to a compound of formula (I), wherein R¹ is selected from the group R1g, consisting of F;
and substituents A, R², R³ and R⁴ are defined as in any of the preceding embodiments.

Another embodiment of the present invention relates to a compound of formula (I), wherein R² is R2A selected from the group R2e, consisting of H, halo, hydroxy, C₁₋₄-alkyl, C₂₋₄-alkynyl, C₃₋₄-cycloalkyl, F₁₋₃-fluoro-C₁₋₄ alkyl, HO—C₁₋₄-alkyl, C₁₋₄-alkyloxy, C₁₋₄-alkyl-O—H₂CH₂C—O—, C₃₋₄-cycloalkyloxy, C₃₋₄-cycloalkyl-H₂C—O—, F₁₋₃-fluoro-C₁₋₄-alkyloxy, C₁₋₄-alkyl-O—C(O)—, H₂N—C(O)—, and C₁₋₄-alkyl-NH—C(O)—;

or R2A is selected from the group R2b, consisting of phenyl, benzyl, phenoxy and benzyloxy, wherein R2b is substituted with one or two R⁴.
or R2A is R2c, which is a 5- or 6-membered mono-heteroaryl ring containing one or two heteroatom members selected from the group consisting of nitrogen, oxygen and sulphur, wherein R2c is substituted with one or two R⁴.
or R2A is R2d, consisting of

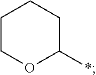

and substituents A, R¹, R³ and R⁴ are defined as in any of the preceding embodiments.

Another embodiment of the present invention relates to a compound of formula (I), wherein R² is R2B selected from the group R2e, consisting of H, halo, hydroxy, C₁₋₄-alkyl, C₂₋₄-alkynyl, C₃₋₄-cycloalkyl, F₁₋₃-fluoro-C₁₋₄ alkyl, HO—C₁₋₄-alkyl, C₁₋₄-alkyloxy, C₁₋₄-alkyl-O—H₂CH₂C—O—, C₃₋₄-cycloalkyloxy, C₃₋₄-cycloalkyl-H₂C—O—, F₁₋₃-fluoro-C₁₋₄-alkyloxy, C₁₋₄-alkyl-O—C(O)—, H₂N—C(O)—, and C₁₋₄-alkyl-NH—C(O)—;
or R2B is selected from the group R2b, consisting of phenyl, benzyl, phenoxy and benzyloxy, wherein R2b is substituted with one or two R⁴;
or R2B is R2f, which is a 5-membered mono-heteroaryl ring containing one or two heteroatom members selected from the group consisting of nitrogen and oxygen, wherein R2f is substituted with one or two R⁴;
or R2B is R2d, consisting of

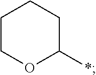

and substituents A, R¹, R³ and R⁴ are defined as in any of the preceding embodiments.

Another embodiment of the present invention relates to a compound of formula (I), wherein R² is R2C selected from the group R2e, consisting of H, halo, hydroxy, C₁₋₄-alkyl, C₂₋₄-alkynyl, C₃₋₄-cycloalkyl, F₁₋₃-fluoro-C₁₋₄ alkyl, HO—C₁₋₄-alkyl, C₁₋₄-alkyloxy, C₁₋₄-alkyl-O—H₂CH₂C—O—, C₃₋₄-cycloalkyloxy, C₃₋₄-cycloalkyl-H₂C—O—, F₁₋₃-fluoro-C₁₋₄-alkyloxy, C₁₋₄-alkyl-O—C(O)—, H₂N—C(O)—, and C₁₋₄-alkyl-NH—C(O)—;
or R2C is selected from the group R2b, consisting of phenyl, benzyl, phenoxy and benzyloxy, wherein R2b is substituted with one or two R⁴;
or R2C is R2g, which is selected from the group consisting of

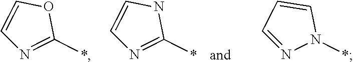

wherein R2g is substituted with one or two R⁴;
or R2C is R2d, consisting of

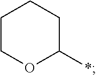

and substituents A, R¹, R³ and R⁴ are defined as in any of the preceding embodiments.

Another embodiment of the present invention relates to a compound of formula (I), wherein R² is R2D selected from the group R2h, consisting of H, F, Cl, hydroxy, C₁₋₄-alkyl, H₃C-alkynyl, C₃₋₄-cycloalkyl, F₁₋₃-fluoro-C₁₋₄ alkyl, HO—C₁₋₄-alkyl, C₁₋₄-alkyloxy, C₁₋₄-alkyl-O—H₂CH₂C—O—, C₃₋₄-cycloalkyloxy, C₃₋₄-cycloalkyl-H₂C—O—, F₁₋₃-fluoro-C₁_₄-alkyloxy, C₁₋₄-alkyl-O—C(O)—, H₂N—C(O)—, and C₁₋₄-alkyl-NH—C(O)—;
or R2D is selected from the group R2b, consisting of phenyl, benzyl, phenoxy and benzyloxy, wherein R2b is substituted with one or two R⁴;
or R2D is R2g, which is selected from the group consisting of

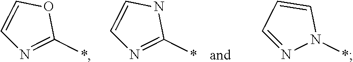

wherein R2g is substituted with one or two R⁴;
or R2D is R2d, consisting of

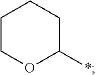

and substituents A, R¹, R³ and R⁴ are defined as in any of the preceding embodiments.

Another embodiment of the present invention relates to a compound of formula (I), wherein R² is R2E selected from the group R2i, consisting of H, F, Cl, hydroxy, methyl, t-butyl, H₃C-alkynyl, cyclopropyl, F₃C—, F₃CCH₂—, F₂CHCH₂—, F₃C—C(CH₃)₂—, HO—CH₂—, H₃C—O—, (H₃C)₂CH—O—, H₃C—O—H₂CH₂C—O—, F₂HC—O—, F₃C—O—, H₃C—O—C(O)—, H₂N—C(O)—, H₃C—NH—C(O)—,

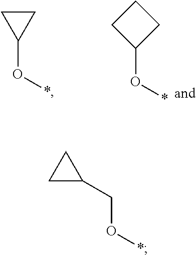

or R2E is selected from the group R2j, consisting of phenyl, m-chlorophenyl, benzyl, phenoxy and benzyloxy;
or R2E is R2g, which is selected from the group consisting of

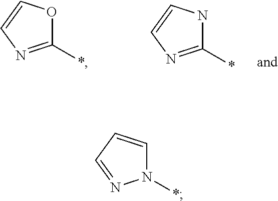

wherein R2g is substituted with H or methyl;
or R2E is R2d, consisting of

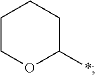

and substituents A, R¹ and R³ are defined as in any of the preceding embodiments.

Another embodiment of the present invention relates to a compound of formula (I), wherein R² is R2F selected from the group R2i, consisting of H, F, Cl, hydroxy, methyl, tis butyl, H₃C-alkynyl, cyclopropyl, F₃C—, F₃CCH₂—, F₂CHCH₂—, F₃C—C(CH₃)₂—, HO—CH₂—, H₃C—O—, (H₃C)₂CH—O—, H₃C—O—H₂CH₂C—O—, F₂HC—O—, F₃C—O—, H₃C—O—C(O)—, H₂N—C(O)—, H₃C—NH—C(O)—,

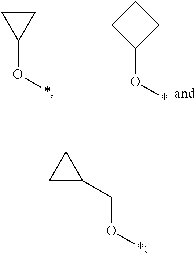

or R2F is selected from the group R2j, consisting of phenyl, m-chlorophenyl, benzyl, phenoxy and benzyloxy;
or R2F is R2g, which is selected from the group consisting of

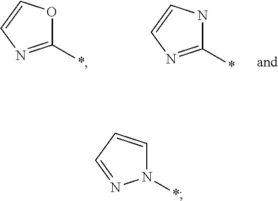

wherein R2g is substituted with H or methyl;
or R2F is R2d, consisting of

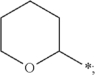

and substituents A, R¹ and R³ are defined as in any of the preceding embodiments.

Another embodiment of the present invention relates to a compound of formula (I), wherein R² is R2G selected from the group R2k, consisting of H, F, Cl, methyl, t-butyl, F₃C—, F₃C—C(CH₃)₂—, H₃C—O—, F₂HC—O— and

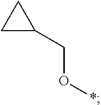

or R2G is selected from the group R2b, consisting of phenyl, benzyl, phenoxy and benzyloxy, wherein R2b is substituted with one or two R⁴;
and substituents A, R¹, R³ and R⁴ are defined as in any of the preceding embodiments.

Another embodiment of the present invention relates to a compound of formula (I), wherein R² is R2H selected from the group R2k, consisting of H, F, Cl, methyl, t-butyl, F₃C—, F₃C—C(CH₃)₂—, H₃C—O—, F₂HC—O— and

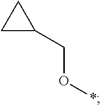

or R2H is selected from the group R2j, consisting of phenyl, m-chlorophenyl, benzyl, phenoxy and benzyloxy;
and substituents A, R¹ and R³ are defined as in any of the preceding embodiments.

Another embodiment of the present invention relates to a compound of formula (I), wherein R² is R2J selected from the group R2m, consisting of Cl, methyl, H₃C—O—, F₂HC—O— and

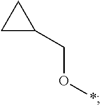

and substituents A, R¹ and R³ are defined as in any of the preceding embodiments.

Another embodiment of the present invention relates to a compound of formula (I), wherein R² is R2K selected from the group R2n, consisting of t-butyl, F₃C— and F₃CC(CH₃)₂—;
and substituents A, R¹ and R³ are defined as in any of the preceding embodiments.

Another embodiment of the present invention relates to a compound of formula (I), wherein R² is R2L selected from H and the group R2b, consisting of phenyl, benzyl, phenoxy and benzyloxy, wherein R2b is substituted with one or two R⁴;
and substituents A, R¹, R³ and R⁴ are defined as in any of the preceding embodiments.

Another embodiment of the present invention relates to a compound of formula (I), wherein R² is R2M selected from the group R2p, consisting of phenyl and m-chlorophenyl;
and substituents A, R¹ and R³ are defined as in any of the preceding embodiments.

Another embodiment of the present invention relates to a compound of formula (I), wherein R² is R2N selected from H;
and substituents A, R¹ and R³ are defined as in any of the preceding embodiments.

Another embodiment of the present invention relates to a compound of formula (I), wherein R³ is selected from the group R3b, consisting of H, methyl and F;
and substituents A, R¹, R² and R⁴ are defined as in any of the preceding embodiments.

Another embodiment of the present invention relates to a compound of formula (I), wherein R³ is selected from the group R3c, consisting of H;
and substituents A, R¹, R² and R⁴ are defined as in any of the preceding embodiments.

Another embodiment of the present invention relates to a compound of formula (I), wherein R³ is selected from the group R3d, consisting of methyl;
and substituents A, R¹, R² and R⁴ are defined as in any of the preceding embodiments.

Another embodiment of the present invention relates to a compound of formula (I), wherein R³ is selected from the group R3e, consisting of F; and substituents A, R¹, R² and R⁴ are defined as in any of the preceding embodiments.

Another embodiment of the present invention relates to a compound of formula (I), wherein R⁴ is selected from the group R4b, consisting of H, methyl, Cl and F;
and substituents A, R¹, R² and R³ are defined as in any of the preceding embodiments.

Another embodiment of the present invention relates to a compound of formula (I), wherein R⁴ is selected from the group R4c, consisting of H;
and substituents A, R¹, R² and R³ are defined as in any of the preceding embodiments.

Another embodiment of the present invention relates to a compound of formula (I), wherein R⁴ is selected from the group R4d, consisting of methyl;
and substituents A, R¹, R² and R³ are defined as in any of the preceding embodiments.

Another embodiment of the present invention relates to a compound of formula (I), wherein R⁴ is selected from the group R4e, consisting of F;
and substituents A, R¹, R² and R³ are defined as in any of the preceding embodiments.

Another embodiment of the present invention relates to a compound of formula (I), wherein R⁴ is selected from the group R4f, consisting of Cl—;
and substituents A, R¹, R² and R³ are defined as in any of the preceding embodiments.

Another embodiment of the present invention relates to a compound of formula (I) above, having formula (I-a)

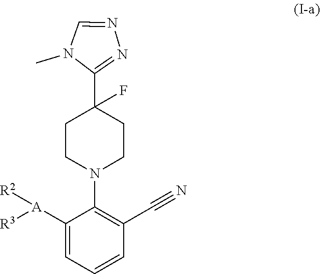

wherein substituents A, R², R³ and R⁴ are defined as in any of the preceding embodiments.

Another embodiment of the present invention relates to a compound of formula (I) above, having formula (I-b)

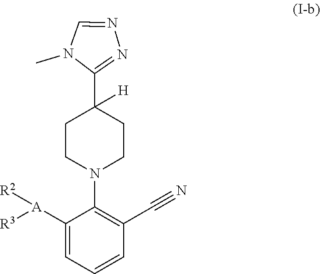

wherein substituents A, R², R³ and R⁴ are defined as in any of the preceding embodiments.

Another embodiment of the present invention relates to a compound of formula (I) above, having formula (I-c)

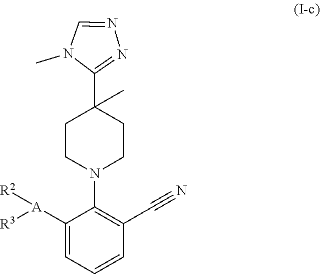

wherein substituents A, R², R³ and R⁴ are defined as in any of the preceding embodiments.

Another embodiment of the present invention relates to a compound of formula (I) above, having formula (I-d)

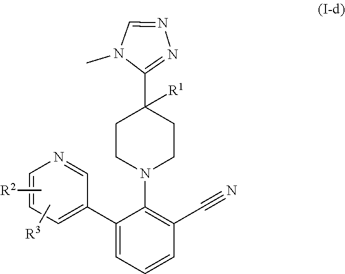

wherein substituents R¹, R², R³ and R⁴ are defined as in any of the preceding embodiments.

Another embodiment of the present invention relates to a compound of formula (I) above, having formula (I-e)

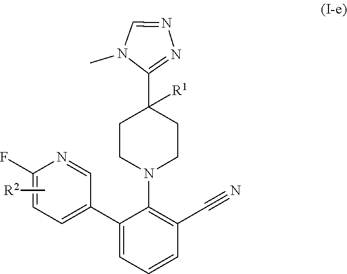

wherein substituents R¹, R² and R⁴ are defined as in any of the preceding embodiments.

Another embodiment of the present invention relates to a compound of formula (I) above, having formula (I-f)

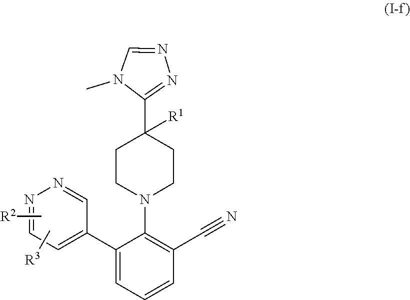

wherein substituents R¹, R², R³ and R⁴ are defined as in any of the preceding embodiments.

Another embodiment of the present invention relates to a compound of formula (I) above, having formula (I-g)

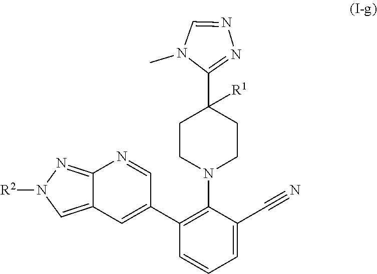

wherein substituents R¹, R² and R⁴ are defined as in any of the preceding embodiments.

Another embodiment of the present invention relates to a compound of formula (I) above, having formula (I-h)

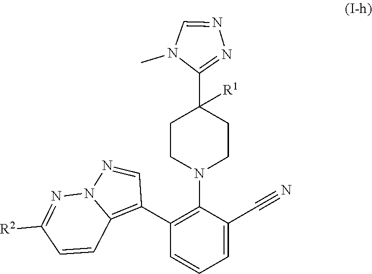

wherein substituents R¹, R² and R⁴ are defined as in any of the preceding embodiments.

Further preferred embodiments of the compounds of formula (I) are encompassed as embodiments (EMB-1) to (EMB-20) in the following Table 1, wherein the substituent definitions above are employed.

TABLE 1
further preferred embodiments

| | Substituent | | | | | |
|---|---|---|---|---|---|---|
| Embodiment | formula | A | R¹ | R² | R³ | R⁴ |
| EMB-1 | I | A7 | R1d | R2K | R3b | — |
| EMB-2 | I | A8 | R1d | R2K | R3b | — |
| EMB-3 | I | A12 | R1d | R2K | R3c | — |
| EMB-4 | I | A7 | R1g | R2K | R3b | — |
| EMB-5 | I | A8 | R1g | R2K | R3b | — |
| EMB-6 | I | A12 | R1g | R2K | R3b | — |
| EMB-7 | I | A7 | R1g | R2K | R3c | — |
| EMB-8 | I | A8 | R1g | R2K | R3c | — |
| EMB-9 | I | A12 | R1g | R2K | R3c | — |
| EMB-10 | I | A9 | R1d | R2G | R3e | — |
| EMB-11 | I | A10 | R1d | R2G | R3e | — |
| EMB-12 | I | A9 | R1g | R2G | R3e | — |
| EMB-13 | I | A10 | R1g | R2G | R3e | — |
| EMB-14 | I-b | A7 | — | R2A | R3b | R4b |
| EMB-15 | I-c | A9 | — | R2N | R3e | — |
| EMB-16 | I-d | — | R1d | R2D | R3b | R4b |
| EMB-17 | I-e | — | R1d | R2J | — | — |
| EMB-18 | I-f | — | R1d | R2N | R3c | — |
| EMB-19 | I-g | — | R1d | R2K | — | — |
| EMB-20 | I-h | — | R1d | R2L | — | R4a |

For example, compounds of the embodiment EMB-1 have for R¹ the genus group R1d as defined above, in combination with the other genus groups for the other substituents in formula (I) as defined within the same row of the table. The same applies analogously to the other variables incorporated in the general formulae.

Particularly preferred is the compound according to formula (I) selected from the group consisting of

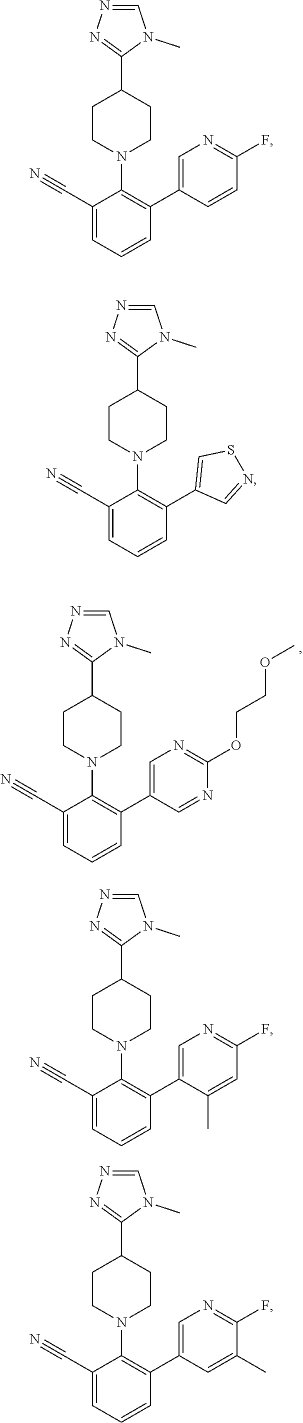

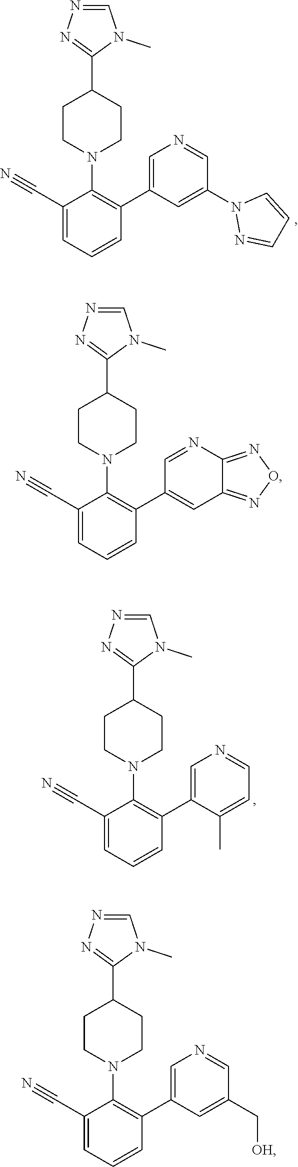 [SMILES: Cn1cnnc1C1CCN(c2c(C#N)cccc2-c2cncc(-n3cccn3)c2)CC1 ; Cn1cnnc1C1CCN(c2c(C#N)cccc2-c2cnc3nonc3c2)CC1 ; Cc1ccncc1-c1cccc(C#N)c1N1CCC(c2nncn2C)CC1 ; Cn1cnnc1C1CCN(c2c(C#N)cccc2-c2cncc(CO)c2)CC1]

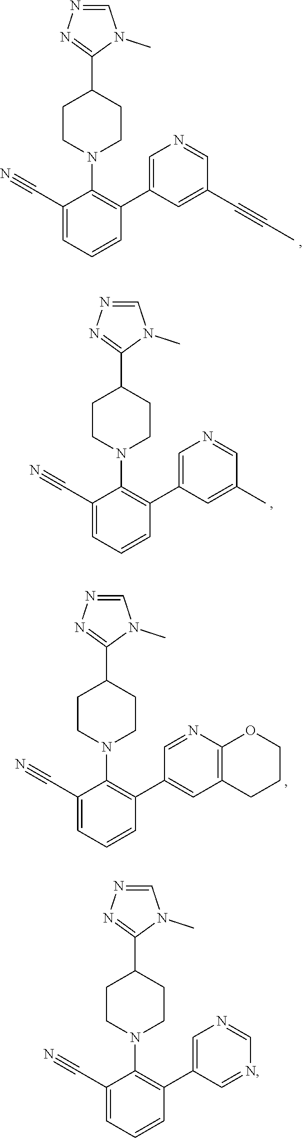

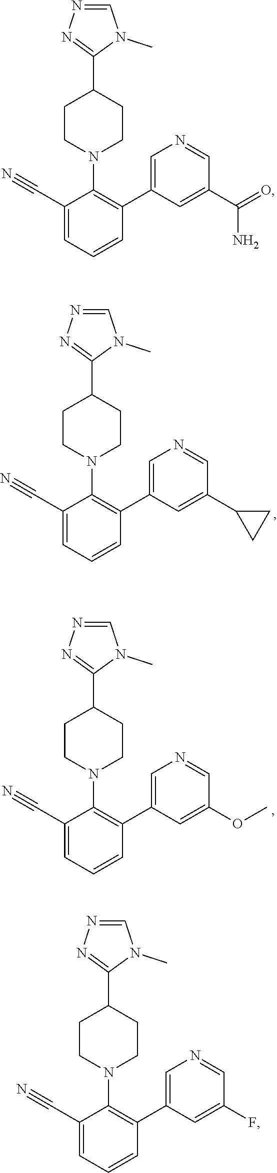 [SMILES: Cn1cnnc1C1CCN(c2c(C#N)cccc2-c2cncc(C(N)=O)c2)CC1 ; Cn1cnnc1C1CCN(c2c(C#N)cccc2-c2cncc(C3CC3)c2)CC1 ; COc1cncc(-c2cccc(C#N)c2N2CCC(c3nncn3C)CC2)c1 ; Cn1cnnc1C1CCN(c2c(C#N)cccc2-c2cncc(F)c2)CC1]

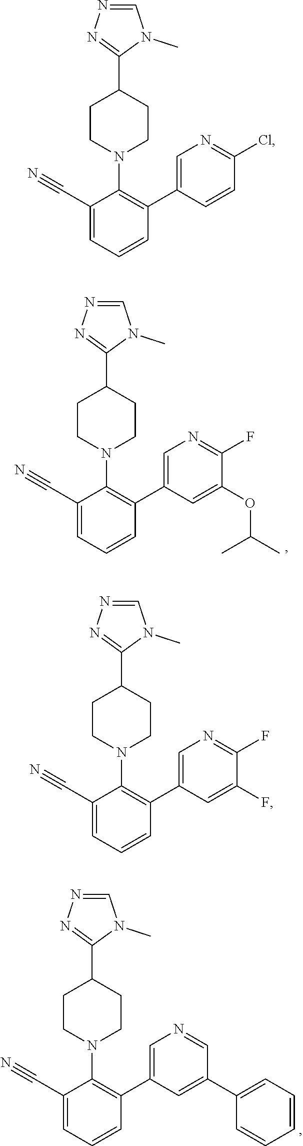

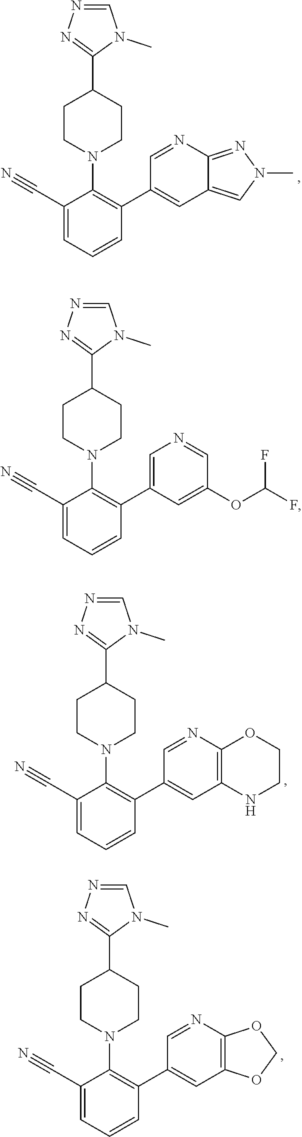

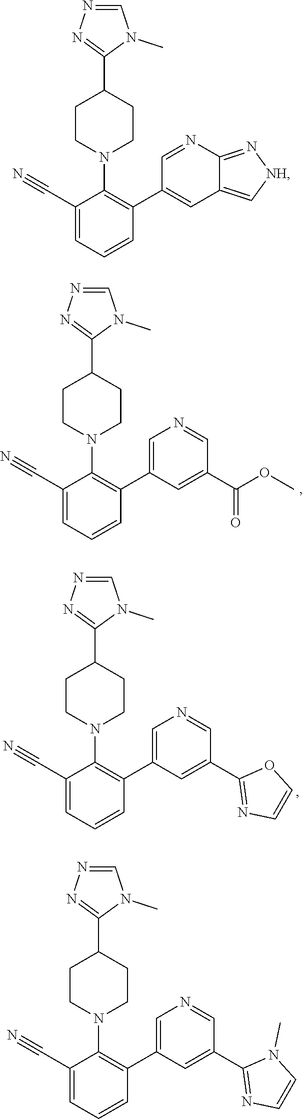

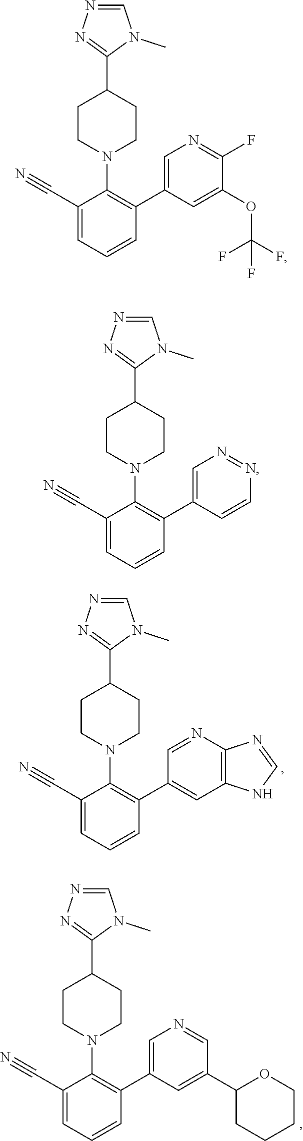

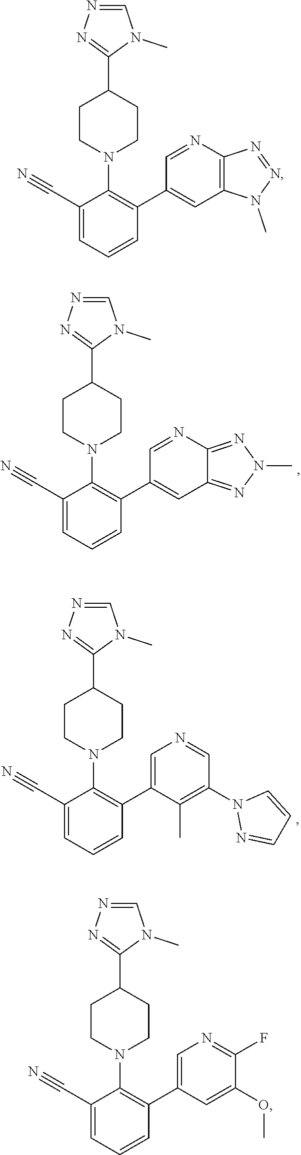

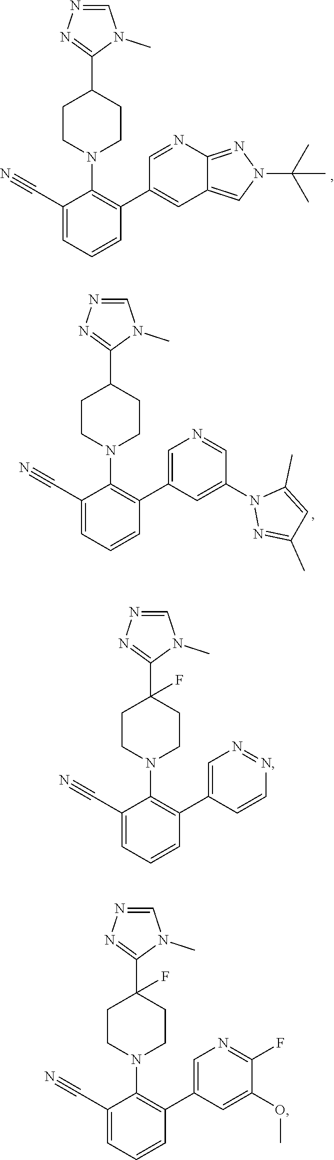

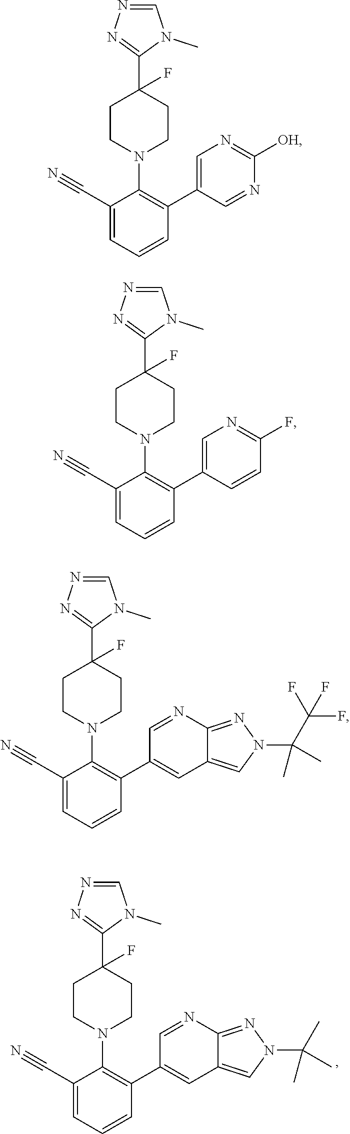

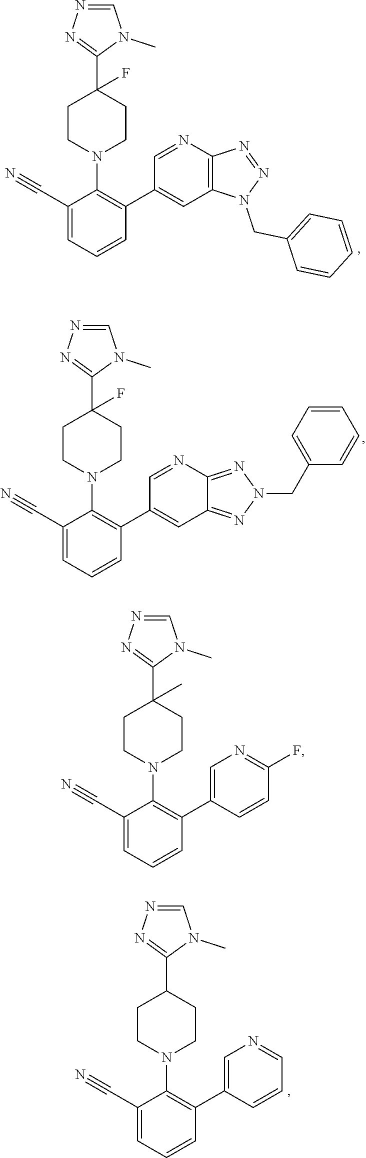

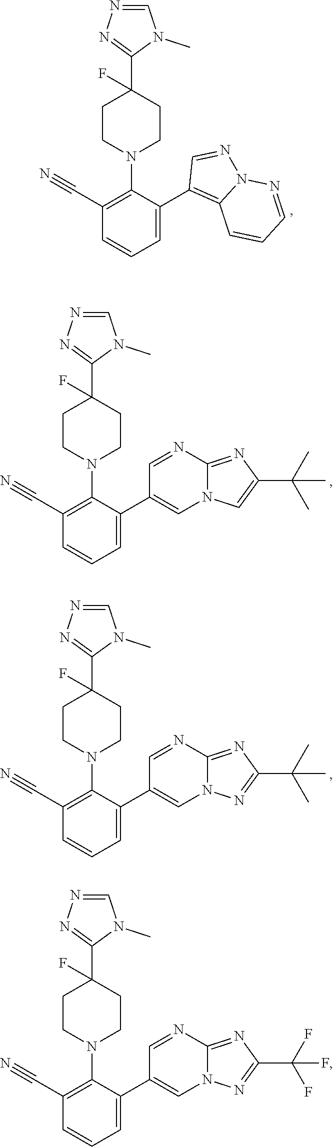

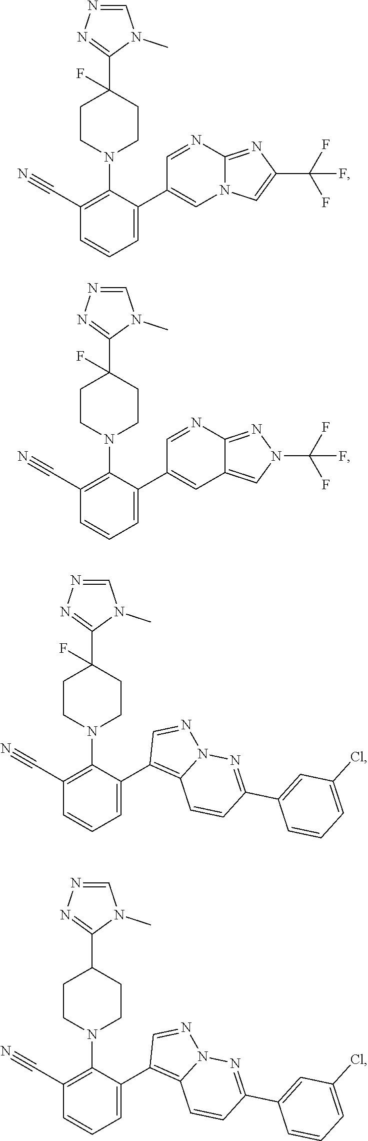

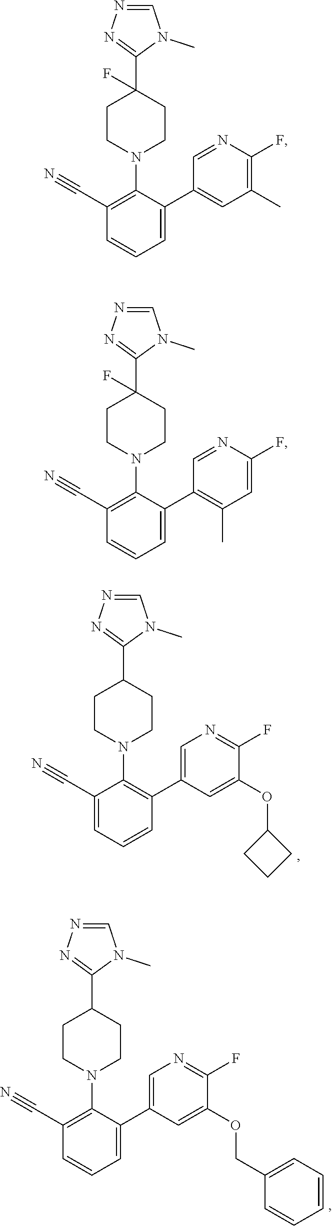

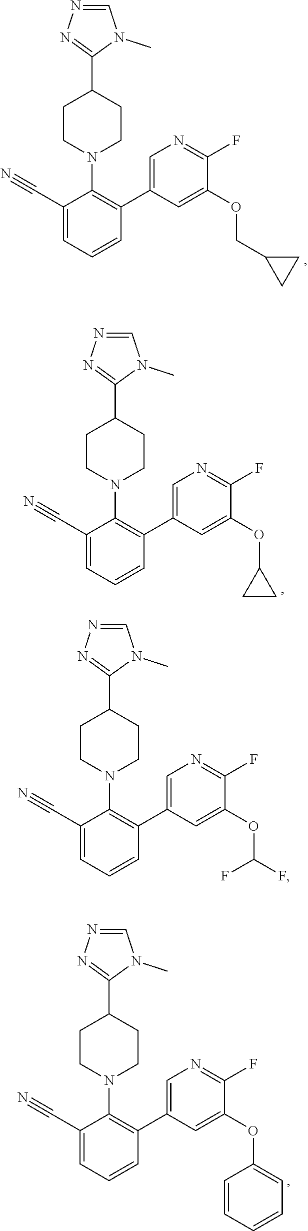

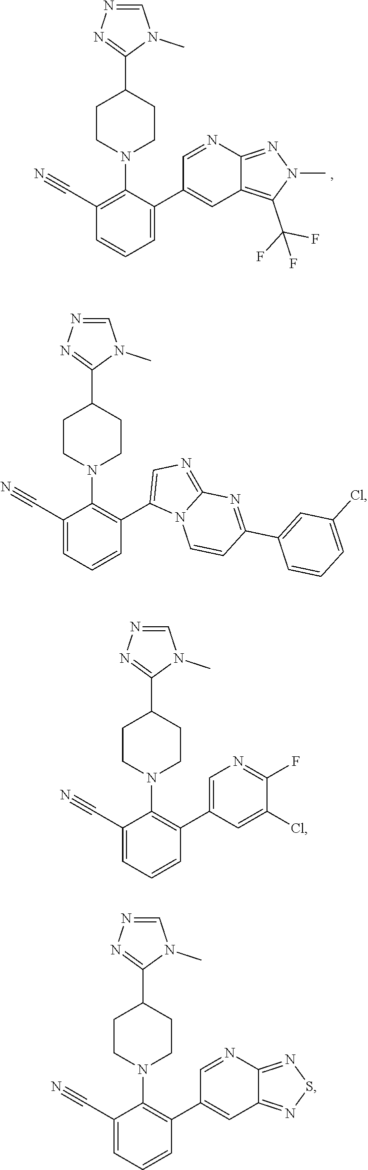

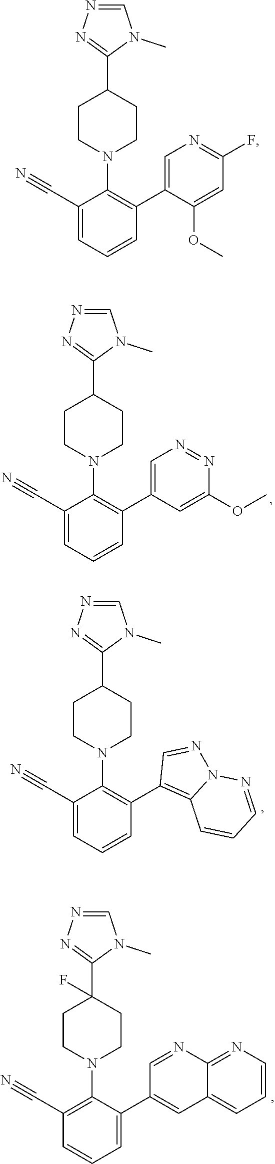

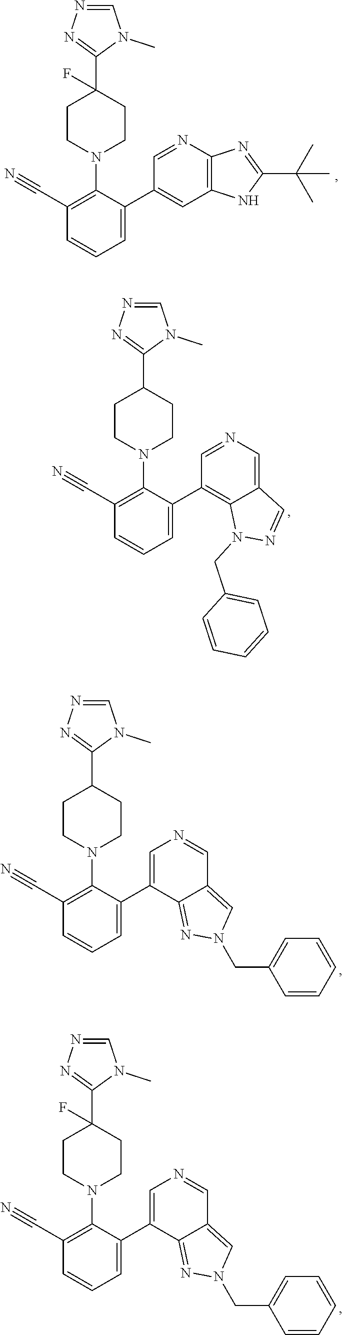 [SMILES: Cn1cnnc1C1(F)CCN(c2c(C#N)cccc2-c2cnc3nc(C(C)(C)C)[nH]c3c2)CC1 ; Cn1cnnc1C1CCN(c2c(C#N)cccc2-c2cncc3cnn(Cc4ccccc4)c23)CC1 ; Cn1cnnc1C1CCN(c2c(C#N)cccc2-c2cncc3cn(Cc4ccccc4)nc23)CC1 ; Cn1cnnc1C1(F)CCN(c2c(C#N)cccc2-c2cncc3cn(Cc4ccccc4)nc23)CC1]

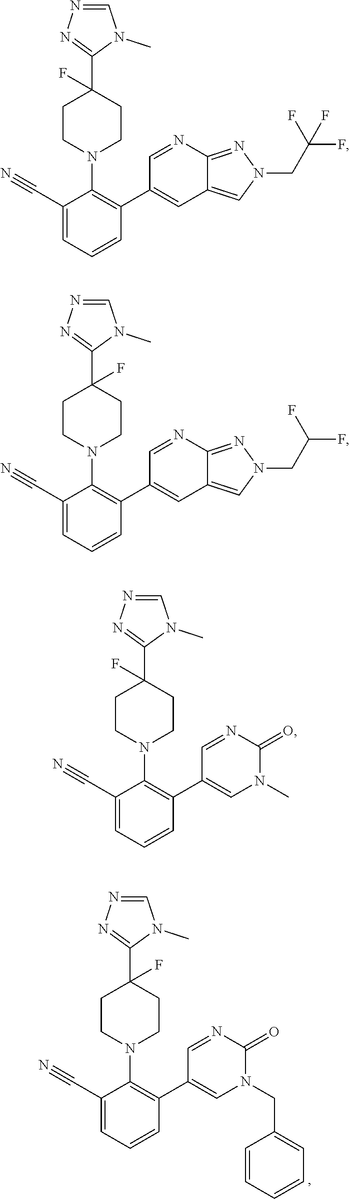

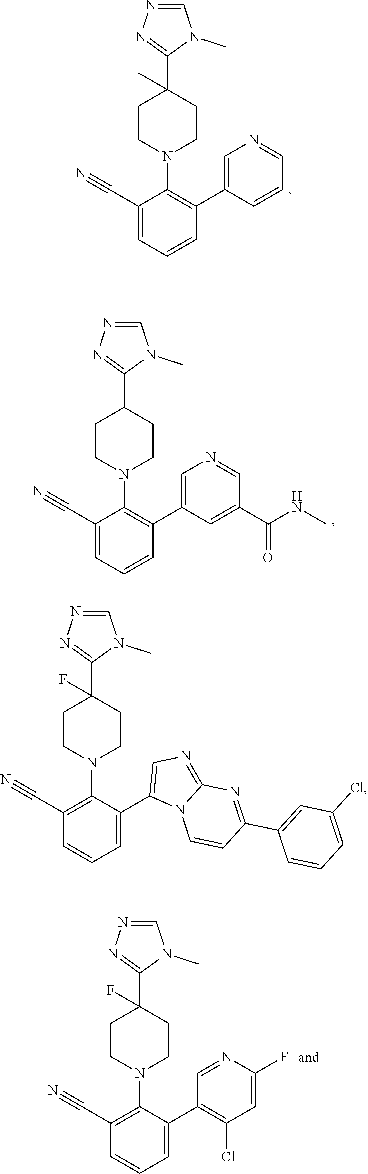

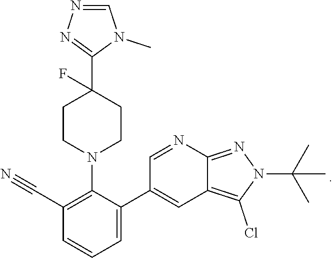 [SMILES: Cn1cnnc1C1(F)CCN(c2c(C#N)cccc2-c2cnc3nn(C(C)(C)C)c(Cl)c3c2)CC1]

Particularly preferred is the compound according to formula (I) selected from the group consisting of example 1, example 5, example 20, example 36, example 37, example 42, example 44, example 49, example 52, example 53, example 56, example 61, example 62, example 66, example 70, example 75, example 79 and example 81, as described hereinafter in EXAMPLES.

Particularly preferred is the compound according to formula (I) selected from the group consisting of example 1, example 5, example 36, example 37, example 42, example 44, example 49, example 52, example 53, example 61, example 62, example 70, example 75, example 79 and example 81, as described hereinafter in EXAMPLES.

The present invention provides novel phenylpiperidine derivatives of formula (I) that are surprisingly potent QPCT/L inhibitors.

Another aspect of the invention refers to compounds according to formula (I) as surprisingly having potent inhibition of QPCT/L in cells relevant for, but not limited to, lung diseases or cancer.

Another aspect of the invention refers to compounds according to formula (I) as surprisingly cellular potent QPCT/L inhibitors having appropriate membrane permeability and low in vitro efflux.

Another aspect of the invention refers to pharmaceutical compositions, containing at least one compound according to formula (I) optionally together with one or more inert carriers and/or diluents.

A further aspect of the present invention refers to compounds according to formula (I), for the use in the prevention and/or treatment of disorders associated with QPCT/L inhibition.

Another aspect of the invention refers to processes of manufacture of the compounds of the present invention.

Further aspects of the present invention will become apparent to the skilled artisan directly from the foregoing and following description and the examples.

### Used Terms and Definitions

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached.

In case a compound of the present invention is depicted in the form of a chemical name and as a formula, in case of any discrepancy the formula shall prevail. An asterisk may be used in sub-formulas to indicate the bond which is connected to the core molecule as defined.

The numeration of the atoms of a substituent starts with the atom which is closest to the core or to the group to which the substituent is attached.

For example, the term “3-carboxypropyl-group” represents the following substituent:

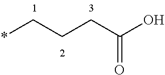 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

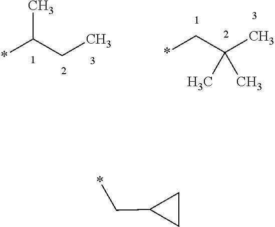

The asterisk may be used in sub-formulas to indicate the bond which is connected to the core molecule as defined.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

The term halogen denotes fluorine, chlorine, bromine and iodine.

The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₂-m-alkynyl” is used for a group “C₂₋ₘ-alkyl” wherein m is an integer selected from 3, 4, 5 or 6, preferably 4, 5 or 6, if at least two carbon atoms of said group are bonded to each other by a triple bond.

The term “C₃_k-cycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—.

The term “mono-heteroaryl ring” means a monocyclic aromatic ring system, containing one or more heteroatoms selected from N, O or S, consisting of 5 to 6 ring atoms.

The term “mono-heteroaryl ring” is intended to include all the possible isomeric forms. Thus, the term “mono-heteroaryl ring” includes the following exemplary structures (not depicted as radicals as each form is optionally attached through a covalent bond to any atom so long as appropriate valences are maintained):

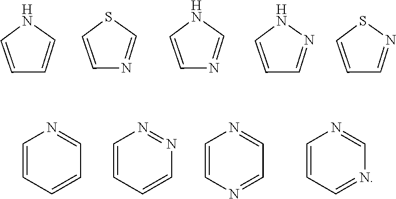

The term “fused bicyclic-heteroaryl ring” means a bicyclic aromatic ring system, containing one or more heteroatoms selected from N, O or S, consisting of 9 to 10 ring atoms. The term “fused bicyclic-heteroaryl ring” is intended to include all the possible isomeric forms. Thus, the term “bicyclic-heteroaryl ring” includes the following exemplary structures (not depicted as radicals as each form is optionally attached through a covalent bond to any atom so long as appropriate valences are maintained):

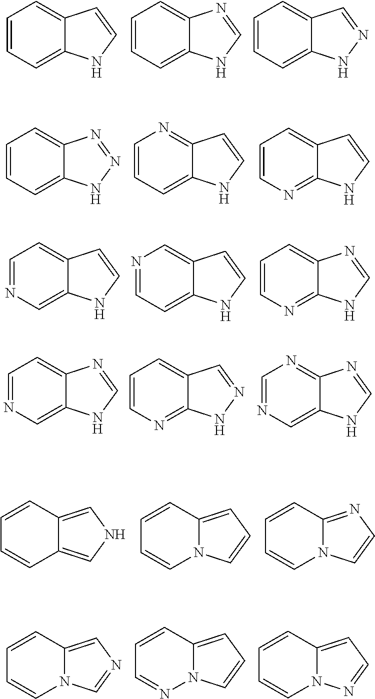

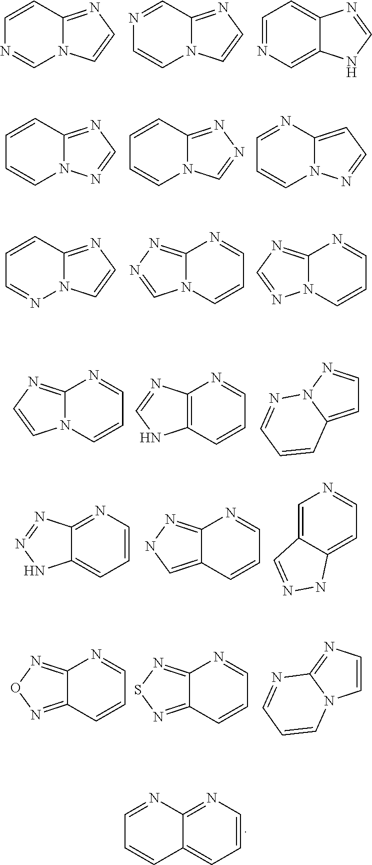

The term pyridinyl refers to the radical of the following ring:

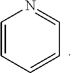

The term pyridazinyl refers to the radical of the following ring:

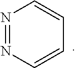

The term pyrimidyl refers to the radical of the following ring:

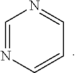

The term 1,2-dihydropyrimidin-2-onyl refers to the radical of the following ring:

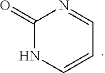

The term pyrazolyl refers to the radical of the following ring:

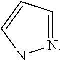

The term thiazolyl refers to the radical of the following ring:

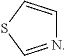

The term isothiazolyl refers to the radical of the following ring:

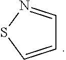

The term oxazolyl refers to the radical of the following ring:

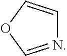

The term isoxazolyl refers to the radical of the following ring:

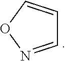

The term 3H-imidazo[4,5-b]pyridinyl refers to the radical of the following ring:

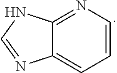

The term imidazo[1,2-a]pyrimidinyl refers to the radical of the following ring:

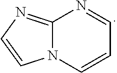

The term 2H-pyrazolo[3,4-b]pyridinyl refers to the radical of the following ring:

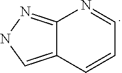

The term 1H-[1,2,3]triazolo[4,5-b]pyridinyl refers to the radical of the following ring:

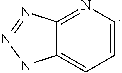

The term [1,2,4]triazolo[4,3-a]pyrimidinyl refers to the radical of the following ring:

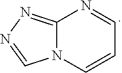

The term 1H-pyrazolo[4,3-c]pyridinyl refers to the radical of the following ring:

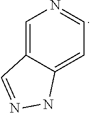

The term [1,2,5]oxadiazolo[3,4-b]pyridinyl refers to the radical of the following ring:

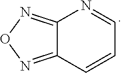

The term [1,2,4]triazolo[1,5-a]pyrimidinyl refers to the radical of the following ring:

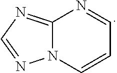

The term [1,2,5]thiadiazolo[3,4-b]pyridinyl refers to the radical of the following ring:

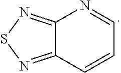

The term 2H-[1,3]dioxolo[4,5-b]pyridinyl refers to the radical of the following ring:

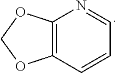

The term imidazo[1,2-a]pyrimidinyl refers to the radical of the following ring:

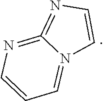

The term pyrazolo[1,5-b]pyridazinyl refers to the radical of the following ring:

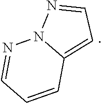

The term 2H,3H,4H-pyrano[2,3-b]pyridinyl refers to the radical of the following ring:

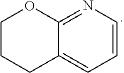

The term 1H,2H,3H-pyrido[2,3-b][1,4]oxazinyl refers to the radical of the following ring:

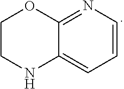

The term 1,8-naphthyridinyl refers to the radical of the following ring:

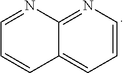

Many of the terms given above may be used repeatedly in the definition of a formula or group and in each case have one of the meanings given above, independently of one another.

### Biological Assays

### Evaluation of Inhibitory Activity on QPCT and QPCTL

### Assay A: Biochemical QPCT and QPCTL Activity Assay

The activity of the compounds of the invention may be demonstrated using the following biochemical enzyme activity assay:

QPCT or QPCTL dependent conversion of N-terminal glutamine to pyroglutamate of CD47 was monitored via MALDI-TOF MS. Test compounds were dissolved in 100% DMSO and serially diluted into clear 1,536-well microtiter plates. Enzymatic reactions were set up in assay buffer containing 20 mM Tris pH 7.5, 0.1 mM TCEP, 0.01% BSA, and 0.001% Tween20. 2.5 μL of 2× concentrated QPCTL (in-house) or QPCT (Origine #TP700028) enzyme in assay buffer (0.5 nM final concentration, columns 1-23) or plain assay buffer (columns 24) were added to each well. The plates were incubated for 10 min in a humidified incubator at 24° C. Subsequently, 2.5 μL of CD47 peptide substrate surrogate (¹⁹QLLFNKTKSVEFTFC³³) was added to each well (final concentration: 10 μM for QPCTL/20 μM for QPCT). The plates were mixed for 30 sec at 1,000 rpm and subsequently incubated for 40 min in a humidified incubator at 24° C. After incubation, the enzymatic reaction was stopped by adding 1 μL containing stable isotope labeled internal standard peptide ¹⁹[Pyr]LLFN(K)TKSVEFTFC³³ (final concentration 4.0 μM) as well as SEN177 (final concentration 10 μM). The plates were sealed with an adhesive foil, mixed for 30 s at 1,000 rpm and stored at room temperature until preparation of the MALDI target plates. MALDI target plates were prepared as described previously.1 Mass spectra were acquired with a rapifleX MALDI-TOF/TOF instrument tracking the signals of the product (¹⁹[Pyr]LLFNKTKSVEFTFC³³, m/z 1,787.9037) as well as internal standard (¹⁹[Pyr]LLFN(K)TKSVEFTFC³³, m/z 1,795.9179) peptide. QPCT or QPCTL activity was monitored by calculating the ratio between product and internal standard signals followed by normalization to high (100% activity) and low (0% activity) controls. Determination of compound potencies was obtained by fitting the dose-response data to a four-parameter logistical equation.

TABLE 2
Biological data for compounds of the invention as obtained in Assay A.

| | Inhibition of | Inhibition of |
|---|---|---|
| | QPCTL: | QPCT: |
| Example | IC₅₀ [nM] | IC₅₀ [nM] |
| 1 | 2 | 7 |
| 2 | 12 | 51 |
| 3 | 11 | 51 |
| 4 | 2 | 12 |
| 5 | 3 | 12 |
| 6 | 1 | 7 |
| 7 | 2 | 12 |
| 8 | 6 | 31 |
| 9 | 11 | 38 |
| 10 | 18 | 75 |
| 11 | 21 | 128 |
| 12 | 12 | 57 |
| 13 | 15 | 52 |
| 14 | 3 | 21 |
| 15 | 28 | 105 |
| 16 | 22 | 86 |
| 17 | 15 | 77 |
| 18 | 56 | 357 |
| 19 | 1 | 9 |
| 20 | 5 | 28 |
| 21 | 50 | 195 |
| 22 | 1 | 3 |
| 23 | 31 | 162 |
| 24 | 14 | 56 |
| 25 | 14 | 84 |
| 26 | 5 | 24 |
| 27 | 15 | 100 |
| 28 | 2 | 12 |
| 29 | 3 | 25 |
| 30 | 1 | 3 |
| 31 | 9 | 71 |
| 32 | 1 | 6 |
| 33 | 1 | 6 |
| 34 | 1 | 6 |
| 35 | 2 | 12 |
| 36 | 1 | 2 |
| 37 | 8 | 28 |
| 38 | 13 | 102 |
| 39 | 5 | 29 |
| 40 | 1 | 4 |
| 41 | 2 | 9 |
| 42 | 4 | 24 |
| 43 | 3 | 28 |
| 44 | 4 | 18 |
| 45 | 4 | 26 |
| 46 | 2 | 13 |
| 47 | 2 | 8 |
| 48 | 7 | 22 |
| 49 | 2 | 9 |
| 50 | 1 | 1 |
| 51 | 2 | 8 |
| 52 | 2 | 3 |
| 53 | 1 | 3 |
| 54 | 0 | 1 |
| 55 | 1 | 1 |
| 56 | 1 | 3 |
| 57 | 5 | 11 |
| 58 | 1 | 1 |
| 59 | 2 | 8 |
| 60 | 0 | 1 |
| 61 | 2 | 1 |
| 62 | 1 | 1 |
| 63 | 1 | 3 |
| 64 | 2 | 5 |
| 65 | 26 | 9 |
| 66 | 3 | 2 |
| 67 | 6 | 24 |
| 68 | 7 | 13 |
| 69 | 0 | 0 |
| 70 | 5 | 2 |
| 71 | 2 | 1 |
| 72 | 1 | 1 |
| 73 | 4 | 8 |
| 74 | 1 | 1 |
| 75 | 1 | 1 |
| 76 | 1 | 5 |
| 77 | 30 | 88 |
| 78 | 34 | 42 |
| 79 | 2 | 3 |
| 80 | 2 | 11 |
| 81 | 1 | 5 |
| 82 | 2 | 3 |
| 83 | 1 | 3 |
| 84 | 3 | 6 |
| 85 | 3 | 4 |
| 86 | 1 | 1 |

TABLE 3
Biological data for prior art compounds as obtained in Assay A.

| | | Inhibition of | Inhibition of |
|---|---|---|---|
| Prior art | | QPCTL: | QPCT: |
| Compound | Reference | IC₅₀ [nM] | IC₅₀ [nM] |
| SEN177 | J-S, NCB 2015; P, | 17 | 79 |
| | JBIC 2018 | | |
| SEN180 | J-S, NCB 2015 | 39 | 176 |
| 1094 | WO 2018/178384 | 111 | 1707 |
| 1095 | WO 2018/178384 | 13 | 260 |
| 3 | WO 2022/086920 | 41 | 454 |
| alternative 3 | WO 2022/086920 | 613 | 2916 |
| 6 | WO 2022/086920 | 1 | 1 |
| 21 | CN 114874186 | 5493 | 1398 |
| 23 | CN 114874186 | 4772 | 1845 |

### Assay B: SIRPα Signalling Assay (Usig Either Raji or A549 Cells)

The activity of the compounds of the invention may be demonstrated using the following SIRPα, signalling assay that measures SIRPα engagement induced by CD47 presented via cell-cell interaction. Two cell types are independently used: the Raji cell line (lymphoblastlike human cell line derived from B lymphocytes from a Burkitt's lymphoma patient in 1963) and A549 cells (adenocarcinomic human alveolar basal epithelial cells).

Test compounds were dissolved in 100% DMSO and serially diluted into a white 384-well microtiter cell culture plate (PerkinElmer #60076780 in case of Raji assay; PDL-coated plates Greiner #781945 in case of A549 assay). 5000 Raji cells (ATCC #CC86) or 5000 A549 cells (ATCC #CCL-185) in Assay Complete Cell Plating reagent 30 (DiscoverX 93-0563R30B) were added per well. The assay plate was incubated for 48 h at 37° C., 95% humidity and 5% CO₂. 15000 reporter cells (Jurkat PathHunter SIRPα V1, DiscoverX #93-1135C19) were added to each well, and the plate was incubated for 5 h at 37° C., 95% humidity and 5% CO₂. Bioassay reagent 1 of the PathHunter Bioassay detection kit (DiscoverX 93-0001) was added to each well of the plate using a multichannel pipette followed by a 15 min incubation at room temperature. Afterwards bioassay reagent 2 was added followed by 60 min incubation at room temperature (incubation in the dark).

The analysis of the data was performed using the luminescence signal generated by beta-galactosidase in the PathHunter reporter cell line. The luminescence measurement was done using a Pherastar Multi-Mode Reader. Dose-response curves & IC₅₀ data were calculated with 4-parameter sigmoidal dose response formula.

TABLE 4
Biological data for compounds of the invention as obtained in Assay B.

| | Inhibition of | Inhibition of |
|---|---|---|
| | SIRα signalling | SIRα signalling |
| | induced by Raji | induced by A549 |
| | cells: | cells: |
| Example | IC₅₀ [nM] | IC₅₀ [nM] |
| 1 | 51 | 9 |
| 2 | 715 | 40 |
| 3 | 629 | 290 |
| 4 | 88 | 13 |
| 5 | 93 | 26 |
| 6 | 220 | 26 |
| 7 | 170 | 67 |
| 8 | 257 | 348 |
| 9 | 309 | — |
| 10 | 692 | 348 |
| 11 | 372 | 436 |
| 12 | 421 | 243 |
| 13 | 782 | 165 |
| 14 | 761 | — |
| 15 | 736 | 303 |
| 16 | 625 | 332 |
| 17 | 1168 | 199 |
| 18 | 2269 | 294 |
| 19 | 53 | 18 |
| 20 | 147 | 53 |
| 21 | 1320 | 530 |
| 22 | 87 | 114 |
| 23 | 1066 | 342 |
| 24 | 368 | 177 |
| 25 | 566 | 339 |
| 26 | 427 | 382 |
| 27 | 483 | — |
| 28 | 250 | 282 |
| 29 | 121 | 81 |
| 30 | 135 | 124 |
| 31 | 244 | 135 |
| 32 | 158 | 69 |
| 33 | 501 | 210 |
| 34 | 52 | 25 |
| 35 | 51 | 18 |
| 36 | 19 | 10 |
| 37 | 325 | 113 |
| 38 | 372 | 98 |
| 39 | 133 | 18 |
| 40 | 214 | 109 |
| 41 | 88 | 97 |
| 42 | 105 | 72 |
| 43 | 342 | 271 |
| 44 | 109 | 121 |
| 45 | 140 | 173 |
| 46 | 97 | 71 |
| 47 | 77 | 53 |
| 48 | 446 | 674 |
| 49 | 63 | 56 |
| 50 | 21 | 33 |
| 51 | 148 | 52 |
| 52 | 67 | 16 |
| 53 | 81 | 22 |
| 54 | 851 | 205 |
| 55 | 38 | 44 |
| 56 | 29 | 7 |
| 57 | 442 | 244 |
| 58 | 493 | 249 |
| 59 | 746 | 619 |
| 60 | 17 | 16 |
| 61 | 23 | 22 |
| 62 | 21 | 11 |
| 63 | 13 | 9 |
| 64 | 61 | 19 |
| 65 | 1036 | — |
| 66 | 100 | 14 |
| 67 | 229 | 58 |
| 68 | 366 | 274 |
| 69 | 11 | 10 |
| 70 | 79 | 74 |
| 71 | 49 | 27 |
| 72 | 28 | 20 |
| 73 | 261 | 122 |
| 74 | 18 | 26 |
| 75 | 7 | 7 |
| 76 | 40 | 46 |
| 77 | 374 | 1865 |
| 78 | 456 | 440 |
| 79 | 16 | 22 |
| 80 | 336 | 176 |
| 81 | 27 | 27 |
| 82 | 63 | 17 |
| 83 | 102 | 47 |
| 84 | 91 | 63 |
| 85 | 70 | 80 |
| 86 | 13 | 23 |

TABLE 5
Biological data for prior art compounds as obtained in Assay B.

| | | Inhibition of | Inhibition of |
|---|---|---|---|
| | | SIRPα signalling | SIRPα signalling |
| | | induced by Raji | induced by A549 |
| | | cells: | cells: |
| Prior art | | IC₅₀ [nM] | IC₅₀ [nM] |
| SEN177 | J-S, NCB 2015; P, | 1365 | 214 |
| | JBIC 2018 | | |
| SEN180 | J-S, NCB 2015 | 2973 | 868 |
| 1094 | WO 2018/178384 | 3240 | 3059 |
| 1095 | WO 2018/178384 | 2203 | 259 |
| 3 | WO 2022/086920 | 2736 | 1404 |
| alternative 3 | WO 2022/086920 | >10000 | >10000 |
| 6 | WO 2022/086920 | 9 | 14 |
| 21 | CN 114874186 | >10000 | >10000 |
| 23 | CN 114874186 | >10000 | >10000 |

### Evaluation of Permeability

### Assay C: Permeability in CACO-2 Cells

Caco-2 cells (1-2×105 cells/1 cm2 area) are seeded on filter inserts (Costar transwell polycarbonate or PET filters, 0.4 μm pore size) and cultured (DMEM) for 10 to 25 days. Compounds are dissolved in appropriate solvent (like DMSO, 1-20 mM stock solutions). Stock solutions are diluted with HTP-4 buffer (128.13 mM NaCl, 5.36 mM KCl, 1 mM MgSO₄, 1.8 mM CaCl₂, 4.17 mM NaHCO₃, 1.19 mM Na₂HPO₄×7H₂O, 0.41 mM NaH₂PO₄×H₂O, 15 mM HEPES, 20 mM glucose, 0.25% BSA, pH 7.2) to prepare the transport solutions (0.1-300 μM compound, final DMSO<=0.5%). The transport solution (TL) is applied to the apical or basolateral donor side for measuring A-B or B-A permeability (3 filter replicates), respectively. Samples are collected at the start and end of experiment from the donor and at various time intervals for up to 2 hours also from the receiver side for concentration measurement by HPLC-MS/MS or scintillation counting. Sampled receiver volumes are replaced with fresh receiver solution.

$$\\ Efflux ratio ( ER ) = permeability B ‐ A / permeability A ‐ B$$

TABLE 7
Biological data for compounds of the invention as obtained in Assay C.

| | Permeability A-B | |
|---|---|---|
| Example | [10⁻⁶ cm/s] | Efflux Ratio |
| 1 | 28 | 2.4 |
| 2 | 25 | 1.8 |
| 3 | 0.6 | 41.8 |
| 4 | 16 | 4.1 |
| 5 | 23 | 1.6 |
| 6 | 3.9 | 16.9 |
| 7 | 1.5 | 19.3 |
| 8 | 3.9 | 9.5 |
| 9 | — | — |
| 10 | 25 | 2.2 |
| 11 | 8.6 | 7.7 |
| 12 | 2.7 | 23.7 |
| 13 | 1.6 | 11.9 |
| 14 | 0.1 | 50.9 |
| 15 | 10.3 | 8.7 |
| 16 | 4.5 | 12.2 |
| 17 | 7.3 | 7.4 |
| 18 | 13.6 | 5 |
| 19 | 39 | 2 |
| 20 | 22 | 3 |
| 21 | 33 | 2.8 |
| 22 | 0.2 | 50.6 |
| 23 | 10.1 | 7.1 |
| 24 | 1 | 34 |
| 25 | 7.4 | 7.4 |
| 26 | 0.4 | 48.6 |
| 27 | 4.4 | 13.4 |
| 28 | 5.5 | 12.4 |
| 29 | 3.1 | 16.5 |
| 30 | <0.3 | >32 |
| 31 | 39 | 2.2 |
| 32 | 0.3 | 28.1 |
| 33 | <0.1 | >27 |
| 34 | 22 | 3.3 |
| 35 | 24 | 2.4 |
| 36 | 34 | 2.4 |
| 37 | 23 | 2 |
| 38 | 20 | 1.9 |
| 39 | 10 | 5.1 |
| 40 | 0.1 | 100 |
| 41 | 2.9 | 19 |
| 42 | 36 | 2 |
| 43 | 2.2 | 22.3 |
| 44 | 11 | 1.5 |
| 45 | 0.8 | 31.6 |
| 46 | 6.8 | 2.5 |
| 47 | 13 | 6 |
| 48 | 1.1 | 21.8 |
| 49 | 18 | 3.6 |
| 50 | 0.5 | 75.5 |
| 51 | 3.4 | 12.9 |
| 52 | 15 | 1.1 |
| 53 | 35 | 1.3 |
| 54 | 0.1 | 11.1 |
| 55 | 1.4 | 40 |
| 56 | 50 | 0.7 |
| 57 | 3.9 | 11 |
| 58 | <0.3 | >4.3 |
| 59 | 0.4 | 34.1 |
| 60 | 0.5 | 100 |
| 61 | 8 | 8.9 |
| 62 | 4.9 | 19 |
| 63 | 3.3 | 14.5 |
| 64 | 29 | 1.2 |
| 65 | 9.8 | 4.9 |
| 66 | 30 | 1.4 |
| 67 | 5.3 | 7.4 |
| 68 | 0.6 | 47.5 |
| 69 | 0.5 | 56.9 |
| 70 | 14 | 2.4 |
| 71 | 2.7 | 24.4 |
| 72 | 3.9 | 14.4 |
| 73 | 3.3 | 14.8 |
| 74 | 1.7 | 14.1 |
| 75 | 12 | 4 |
| 76 | 2.6 | 22.3 |
| 77 | 0.7 | 52.3 |
| 78 | 6.5 | 7.4 |
| 79 | 40 | 0.7 |
| 80 | 0.2 | 37.8 |
| 81 | 39 | 0.7 |
| 82 | 41 | 1.6 |
| 83 | 55 | 1.3 |
| 84 | 33 | 1.8 |
| 85 | | 1.8 |
| 86 | 18 | 1.6 |

TABLE 8
Biological data for prior art compounds as obtained in Assay C.

| Prior art | | Permeability A-B | |
|---|---|---|---|
| Compound | Reference | [10⁻⁶ cm/s] | Efflux Ratio |
| SEN177 | J-S, NCB 2015; P, | 11.0 | 3.3 |
| | JBIC 2018 | | |
| SEN180 | J-S, NCB 2015 | 4.2 | 7.1 |
| 1094 | WO 2018/178384 | 68 | 0.5 |
| 1095 | WO 2018/178384 | 73 | 1.0 |
| 3 | WO 2022/086920 | 93 | 0.8 |
| alternative 3 | WO 2022/086920 | 97 | 0.7 |
| 6 | WO 2022/086920 | 0.2 | 65.0 |
| 21 | CN 114874186 | 0.7 | 27.3 |
| 23 | CN 114874186 | 3.5 | 17.6 |

### Evaluation of Microsomal Clearance

### Microsomal Clearance:

The metabolic degradation of the test compound was assayed at 37° C. with pooled liver microsomes from various species. The final incubation volume of 60 μl per time point contains TRIS buffer pH 7.6 at room temperature (0.1 M), magnesium chloride (5 mM), microsomal protein (1 mg/mL for human and dog, 0.5 mg/mL for other species) and the test compound at a final concentration of 1 μM. Following a short preincubation period at 37° C., the reactions were initiated by addition of betanicotinamide adenine dinucleotide phosphate, reduced form (NADPH, 1 mM), and terminated by transferring an aliquot into solvent after different time points. After centrifugation (10000 g, 5 min), an aliquot of the supernatant was assayed by LC-MS/MS for the amount of parent compound. The half-life was determined by the slope of the semi-logarithmic plot of the concentration-time profile.

The intrinsic clearance (CL_INTRINSIC) is calculated by considering the amount of protein in the incubation:

$$\\ CL\_INTRINSIC [ µ l / min / mg protein ] = ( Ln 2 / ( half ‐ life [ min ] * protein content [ mg / ml ] ) ) * 1000$$

$$\\ CL\_INTRINSIC \_INVIVO [ ml / min / kg ] = ( CL\_INTRINSIC [ µ l / min / mg protein ] \times MPPGL [ mg protein / g liver ] \times liver factor [ g / kg body weight ] ) / 1000$$

$$\\ Qh [ \% ] = CL [ ml / min / kg ] / hepatic blood flow [ ml / min / kg ] )$$

Hepatocellularity, human: 120×10e6 cells/g liver
Liver factor, human: 25.7 g/kg bodyweight
Blood flow, human: 21 ml/(min×kg)

### Evaluation of Hepatocyte Clearance

### Hepatocyte Clearance

The metabolic degradation of a test compound is assayed in a human hepatocyte suspension. After recovery from cryopreservation, human hepatocytes are diluted in Dulbecco's modified eagle medium (supplemented with 3.5 μg glucagon/500 mL, 2.5 mg insulin/500 mL, 3.75 mg hydrocortisone/500 mL, 5% human serum) to obtain a final cell density of 1.0×10⁶ cells/mL.

Following a 30 minutes preincubation in a cell culture incubator (37° C., 10% CO₂), test compound solution is spiked into the hepatocyte suspension, resulting in a final test compound concentration of 1 μM and a final DMSO concentration of 0.05%.

The cell suspension is incubated at 37° C. (cell culture incubator, horizontal shaker) and samples are removed from the incubation after 0, 0.5, 1, 2, 4 and 6 hours. Samples are quenched with acetonitrile (containing internal standard) and pelleted by centrifugation. The supernatant is transferred to a 96-deepwell plate, and prepared for analysis of decline of parent compound by HPLC-MS/MS.

The percentage of remaining test compound is calculated using the peak area ratio (test compound/internal standard) of each incubation time point relative to the time point 0 peak area ratio. The log-transformed data are plotted versus incubation time, and the absolute value of the slope obtained by linear regression analysis is used to estimate in vitro half-life (T½).

In vitro intrinsic clearance (CLint) is calculated from in vitro T½ and scaled to whole liver using a hepatocellularity of 120×106 cells/g liver, a human liver per body weight of 25.7 g liver/kg as well as in vitro incubation parameters, applying the following equation:

$$\\ CL\_INTRINSIC \_IN VIVO [ mL / min / kg ] = ( CL\_INTRINSIC [ µL / min / 106 cells ] \times hepato ‐ cellularity [ 106 cells / g liver ] \times liver factor [ g / kg body weight ] ) / 1000$$

Hepatic in vivo blood clearance (CL) is predicted according to the well-stirred liver model considering an average liver blood flow (QH) of 20.7 mL/min/kg:

$$\\ CL [ mL / min / kg ] = CL\_INTRINSIC \_IN VIVO [ mL / min / kg ] \times hepatic blood flow [ mL / min / kg ] / ( CL\_INTRINSIC \_IN VIVO [ mL / min / kg ] + hepatic blood flow [ mL / min / kg ] )$$

Results are expressed as percentage of hepatic blood flow:

$$\\ QH [ \% ] = CL [ mL / min / kg ] / hepatic blood flow [ mL / min / kg ] )$$

### Evaluation of Plasma Protein Binding

Equilibrium dialysis technique is used to determine the approximate in vitro fractional binding of test compounds to plasma proteins applying Dianorm Teflon dialysis cells (micro 0.2). Each dialysis cell consists of a donor and an acceptor chamber, separated by an ultrathin semipermeable membrane with a 5 kDa molecular weight cutoff. Stock solutions for each test compound are prepared in DMSO at 1 mM and serially diluted to obtain a final test concentration of 1 μM. The subsequent dialysis solutions are prepared in plasma (supplemented with NaEDTA as anticoagulant), and aliquots of 200 μl test compound dialysis solution in plasma are dispensed into the donor (plasma) chambers. Aliquots of 200 μl dialysis buffer (100 mM potassium phosphate, pH 7.4, supplemented with up to 4.7% Dextran) are dispensed into the buffer (acceptor) chamber. Incubation is carried out for 2 hours under rotation at 37° C. for establishing equilibrium.

At the end of the dialysis period, aliquots obtained from donor and acceptor chambers, respectively, are transferred into reaction tubes and processed for HPLC-MS/MS analysis.

Analyte concentrations are quantified in aliquots of samples by HPLC-MS/MS against calibration curves.

Percent bound is calculated using the formula:

$$\\ \% bound = ( plasma concentration - buffer concentration / plasma concentration ) \times 100$$

### Evaluation of Solubility

Saturated solutions are prepared in well plates (format depends on robot) by adding an appropriate volume of selected aqueous media (typically in the range of 0.25-1.5 ml) into each well which contains a known quantity of solid drug substance (typically in the range 0.5-5.0 mg). The wells are shaken or stirred for a predefined time period (typically in a range of 2-24 h) and then filtered using appropriate filter membranes (typically PTFE-filters with 0.45 μm pore size). Filter absorption is avoided by discarding the first few drops of filtrate. The amount of dissolved drug substance is determined by UV spectroscopy. In addition, the pH of the aqueous saturated solution is measured using a glass-electrode pH meter.

### Evaluation of Metabolism in Human Hepatocytes In Vitro

The metabolic pathway of a test compound is investigated using primary human hepatocytes in suspension. After recovery from cryopreservation, human hepatocytes are incubated in Dulbecco's modified eagle medium containing 5% human serum and supplemented with 3.5 μg glucagon/500 ml, 2.5 mg insulin/500 ml and 3.75 mg/500 ml hydrocortisone.

Following a 30 min preincubation in a cell culture incubator (37° C., 10% CO₂), test compound solution is spiked into the hepatocyte suspension to obtain a final cell density of 1.0*10⁶ to 4.0*10⁶ cells/ml (depending on the metabolic turnover rate of the compound observed with primary human hepatocytes), a final test compound concentration of 10 μM, and a final DMSO concentration of 0.05%.

The cells are incubated for six hours in a cell culture incubator on a horizontal shaker, and samples are removed from the incubation after 0, 0.5, 1, 2, 4 or 6 hours, depending on the metabolic turnover rate. Samples are quenched with acetonitrile and pelleted by centrifugation. The supernatant is transferred to a 96-deepwell plate, evaporated under nitrogen and resuspended prior to bioanalysis by liquid chromatography-high resolution mass spectrometry for identification of putative metabolites.

The structures are assigned tentatively based on Fourier-Transform-MSⁿ data. Metabolites are reported as percentage of the parent in human hepatocyte incubation with a threshold of ≥4%.

### Evaluation of Pharmacokinetic Characteristics

The test compound is administered either intravenously or orally to the respective test species. Blood samples are taken at several time points post application of the test compound, anticoagulated and centrifuged.

The concentration of analytes—the administered compound and/or metabolites—are quantified in the plasma samples. PK parameters are calculated using non compartment methods. AUC and Cmax are normalized to a dose of 1 μmol/kg.

### Method of Treatment

The present invention is directed to compounds of general formula (I) which are useful in the prevention and/or treatment of a disease and/or condition associated with or modulated by QPCT/L activity, including but not limited to the treatment and/or prevention of cancer, fibrotic diseases, neurodegenerative diseases, atherosclerosis, infectious diseases, chronic kidney diseases.

The compounds of general formula (I) are useful for the prevention and/or treatment of:
(1) Pulmonary fibrotic diseases such as pneumonitis or interstitial pneumonitis associated with collagenosis, e g. lupus erythematodes, systemic scleroderma, rheumatoid arthritis, polymyositis and dermatomysitis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-I-antitrypsin deficiency.
(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelotibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiis opsy), air pollution, prior exacerbation and medications.
(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, non-Hodgkin'lymphoma (NHL), urothelial cancer, or peritoneal cancer.
(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.
(5) Neurodegenerative disorders such as amyotrophic lateral sclerosis, multiple sclerosis, Parkinson's disease, Alzheimer's disease, Huntington's disease, multiple system atrophy, or prion diseases.

Accordingly, the present invention relates to a compound of general formula (I) or a pharmaceutically acceptable salt thereof for use as a medicament.

Furthermore, the present invention relates to the use of a compound of general formula (I) for the treatment and/or prevention of a disease and/or condition associated with or modulated by QPCT/L activity.

Furthermore, the present invention relates to the use of a compound of general formula (I) or a pharmaceutically acceptable salt thereof or a pharmaceutical composition thereof for the treatment and/or prevention of cancer, fibrotic diseases, neurodegenerative diseases, atherosclerosis, infectious diseases, chronic kidney diseases.

Furthermore, the present invention relates to the use of a compound of general formula (I) or a pharmaceutically acceptable salt thereof or a pharmaceutical composition thereof for the treatment and/or prevention of: (1) Pulmonary fibrotic diseases such as pneumonitis or interstitial pneumonitis associated with collagenosis, e g. lupus erythematodes, systemic scleroderma, rheumatoid arthritis, polymyositis and dermatomysitis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan'cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-I-antitrypsin deficiency.
(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelotibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiopsy), air pollution, prior exacerbation and medications.
(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, non-Hodgkin'lymphoma (NHL), urothelial cancer, or peritoneal cancer.
(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.
(5) Neurodegenerative disorders such as amyotrophic lateral sclerosis, multiple sclerosis, Parkinson's disease, Alzheimer's disease, Huntington's disease, multiple system atrophy, or prion diseases.

In a further aspect the present invention relates to a compound of general formula (I) or a pharmaceutically acceptable salt thereof or a pharmaceutical composition thereof for use in the treatment and/or prevention of above-mentioned diseases and conditions.

In a further aspect the present invention relates to the use of a compound of general formula (I) or a pharmaceutically acceptable salt thereof or a pharmaceutical composition thereof for the preparation of a medicament for the treatment and/or prevention of above-mentioned diseases and conditions.

In a further aspect of the present invention the present invention relates to methods for the treatment or prevention of above-mentioned diseases and conditions, which method comprises the administration of an effective amount of a compound of general formula (I) or a pharmaceutically acceptable salt thereof or a pharmaceutical composition thereof to a huis man being.

### Combination Therapy

The compounds of the invention may further be combined with one or more, preferably one additional therapeutic agent. According to one embodiment the additional therapeutic agent is selected from the group of therapeutic agents useful in the treatment of diseases or conditions described hereinbefore, in particular associated with cancer, fibrotic diseases, Alzheimer's diseases, atherosclerosis, infectious diseases, chronic kidney diseases and auto-immune disease.

Additional therapeutic agents that are suitable for such combinations include in particular those, which, for example, potentiate the therapeutic effect of one or more active substances with respect to one of the indications mentioned and/or allow the dosage of one or more active substances to be reduced.

Therefore, a compound of the invention may be combined with one or more additional therapeutic agents selected from the group consisting of chemotherapy, targeted cancer therapy, cancer immunotherapy, irradiation, antifibrotic agents, anti-tussive agents, antiinflammatory agents, anti-atopic dermatitis, and broncho dilators.

Chemotherapy is a type of cancer therapy that uses one or more chemical anti-cancer drugs, such as cytostatic or cytotoxic substances, cell proliferation inhibitors, anti-angiogenic substances and the like. Examples include folic acid (Leucovorin), 5-Fluorouracil, Irinotecan, Oxaliplatin, cis-platin Azacytidine, gemcitabine, alkylation agents, antimitotic agents, taxanes and further state-of-the-art or standard-of-care compounds.

Targeted therapy is a type of cancer treatment that uses drugs to target specific genes and proteins that help cancer cells survive and grow. Targeted therapy includes agents such as inhibitors of growth factors (e.g. platelet derived growth factor (PDGF), fibroblast growth factor (FGF), vascular endothelial growth factor (VEGF), epidermal growth factor (EGF), insulin-like growth factors (IGF), human epidermal growth factor (HER, e.g. HER2, HER3, HER4) and hepatocyte growth factor), tyrosine-kinases, KRAS, BRAF, BCR-ABL, mTOR, cyclin-dependent kinases, or MDM2.

Cancer immunotherapy is a type of therapy that uses substances to stimulate or suppress the immune system to help the body fight cancer. Cancer immunotherapy includes a therapeutic antibody, such as: anti-Her2 antibody, an anti-EGFR antibody, and an anti-PDGFR antibody; an anti-GD2 (Ganglioside G2) antibody. Examples include Dinutuximab, Olaratumab, Trastuzumab, Pertuzumab, Ertumaxomab, Cetuximab, Necitumumab, Nimotuzumab, Panitumumab, or rituximab. Cancer immunotherapy also includes a therapeutic antibody which is a checkpoint inhibitor, such as an anti PD1, anti PD-L1 antibody or CTLA4 inhibitor. Examples include Atezolizumab, Avelumab, and Durvalumab, Ipilimumab, nivolumab, or pembrolizumab. Cancer immunotherapy also includes agents which target (inhibit) the CD47-SIRPα signaling axis, such as agents which bind to CD47 or SIRPα. Non-limiting examples include antibodies such as anti-CD47 antibodies and antiSIRPα antibodies, and recombinant Fc-fusion proteins such as CD47-Fc and SIRPα-Fc. Cancer immunotherapy also includes STING-targeting agent, or T cell engagers, such as blinatumomab.

Antifibrotic agents are for example nintedanib, pirfenidone, phosphodiesterase-IV (PDE4) inhibitors such as roflumilast or specific PDE4b inhibitors like BI 1015550, autotaxin inhibitors such as GLPG-1690 or BBT-877; connective tissue growth factor (CTGF) blocking antibodies such as Pamrevlumab; B-cell activating factor receptor (BAFF-R) blocking antibodies such as Lanalumab, alpha-V/beta-6 blocking inhibitors such as BG-00011/STX100, recombinant pentraxin-2 (PTX-2) such as PRM-151; c-Jun-N-terminal kinase (JNK) inhibitors such as CC-90001; galectin-3 inhibitors such as TD-139; G-protein coupled receptor 84 (GPR84) inhibitors; G-protein coupled receptor 84/G-protein coupled receptor dual inhibitors such asPBI-4050, Rho Associated Coiled-Coil Containing Protein Kinase 2 (ROCK2) inhibitors such as KD-025, heat shock protein 47 (HSP47) small interfering RNA such as BMS-986263/ND-L02-s0201; Wnt pathway inhibitor such as SM-04646; LD4/PDE3/4 inhibitors such as Tipelukast; recombinant immuno-modulatory domains of histidyl tRNA synthetase (HARS) such as ATYR-1923, prostaglandin synthase inhibitors such as ZL-2102/SAR-191801; 15-hydroxy-eicosapentaenoic acid (15-HEPE e.g. DS102); Lysyl Oxidase Like 2 (LOXL2) inhibitors such as PAT-1251, PXS-5382/PXS-5338; phosphoinositide 3-kinases (PI3K)/mammalian target of rapamycin (mTOR) dual inhibitors such as HEC-68498; calpain inhibitors such as BLD-2660; mitogen-activated protein kinase kinase kinase (MAP3K19) inhibitors such as MG-S-2525; chitinase inhibitors such as OATD-01,mitogen-activated protein kinase-activated protein kinase 2 (MAPKAPK2) inhibitors such as MMI-0100; transforming growth factor beta I (TGF-beta I) small interfering RNA such as TRKZSO/BNC-1021; or lysophosphatidic acid receptor antagonists such as BMS986278.

The dosage for the combination partners mentioned above is usually ⅕ of the lowest dose normally recommended up to 1/1 of the normally recommended dose.

Therefore, in another aspect, this invention relates to the use of a compound according to the invention in combination with one or more additional therapeutic agents described hereinbefore and hereinafter for the treatment of diseases or conditions which may be affected or which are mediated by QPCT/L, in particular diseases or conditions as described hereinbefore and hereinafter.

In a further aspect this invention relates to a method for treating a disease or condition which can be influenced by the inhibition of QPCT/L in a patient that includes the step of administering to the patient in need of such treatment a therapeutically effective amount of a compound of formula (I) or a pharmaceutically acceptable salt thereof in combination with a therapeutically effective amount of one or more additional therapeutic agents.

In a further aspect this invention relates to the use of a compound of formula (I) or a pharmaceutically acceptable salt thereof in combination with one or more additional therapeutic agents for the treatment of diseases or conditions which can be influenced by the inhibition of QPCT/L in a patient in need thereof.

In yet another aspect the present invention relates to a method for the treatment of a disease or condition mediated by QPCT/L activity in a patient that includes the step of administering to the patient, preferably a human, in need of such treatment a therapeutically effective amount of a compound of the present invention in combination with a therapeutically effective amount of one or more additional therapeutic agents described in hereinbefore and hereinafter.

The use of the compound according to the invention in combination with the additional therapeutic agent may take place simultaneously or at staggered times.

The compound according to the invention and the one or more additional therapeutic agents may both be present together in one formulation, for example a tablet or capsule, or separately in two identical or different formulations, for example as a so-called kit-of-parts.

Consequently, in another aspect, this invention relates to a pharmaceutical composition that comprises a compound according to the invention and one or more additional therapeutic agents described hereinbefore and hereinafter, optionally together with one or more inert carriers and/or diluents.

Other features and advantages of the present invention will become apparent from the following more detailed examples which illustrate, by way of example, the principles of the invention.

### Preparation

The compounds according to the present invention and their intermediates may be obtained using methods of synthesis which are known to the one skilled in the art and described in the literature of organic synthesis. Preferably, the compounds are obtained in analogous fashion to the methods of preparation explained more fully hereinafter, in particular as described in the experimental section. In some cases, the order in carrying out the reaction steps may be varied. Variants of the reaction methods that are known to the one skilled in the art but not described in detail here may also be used.

The general processes for preparing the compounds according to the invention will become apparent to the one skilled in the art studying the following schemes. Any functional groups in the starting materials or intermediates may be protected using conventional protecting groups. These protecting groups may be cleaved again at a suitable stage within the reaction sequence using methods familiar to the one skilled in the art.

The compounds according to the invention are prepared by the methods of synthesis described hereinafter in which the substituents of the general formulae have the meanings given herein before. These methods are intended as an illustration of the invention without restricting its subject matter and the scope of the compounds claimed to these examples. Where the preparation of starting compounds is not described, they are commercially obtainable or may be prepared analogously to known compounds or methods described herein. Substances described in the literature are prepared according to the published methods of synthesis. Abbreviations are as defined in the Examples section.

Compounds of formula (I) may be prepared as shown in Scheme I below.

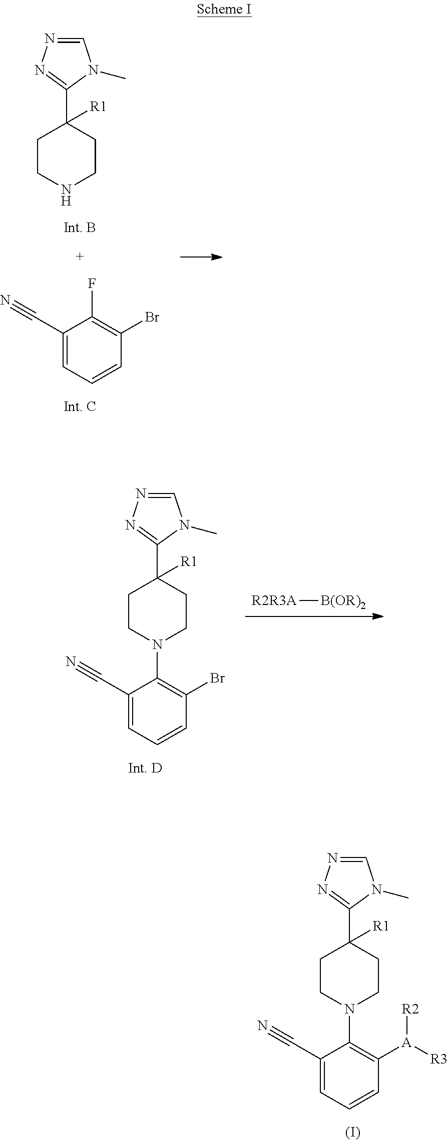

In scheme I, N-methyl triazolyl piperidines (Int. B; R1=H, F, Me) undergo a nucleophilic aromatic substitution with aryl fluoride (Int. C). The reaction is typically run at elevated temperature (100-130° C.). The intermediate (Int. D) is then subjected to a Suzuki-cross coupling with a hetero-aryl boronic acid derivative in the presence of a suitable catalyst (e.g. Pd(dppf)Cl₂)) and a suitable base (e.g. an aqueous K₂CO₃ solution) at elevated temperature (e.g. 100° C.) to afford compounds of general formula (I).

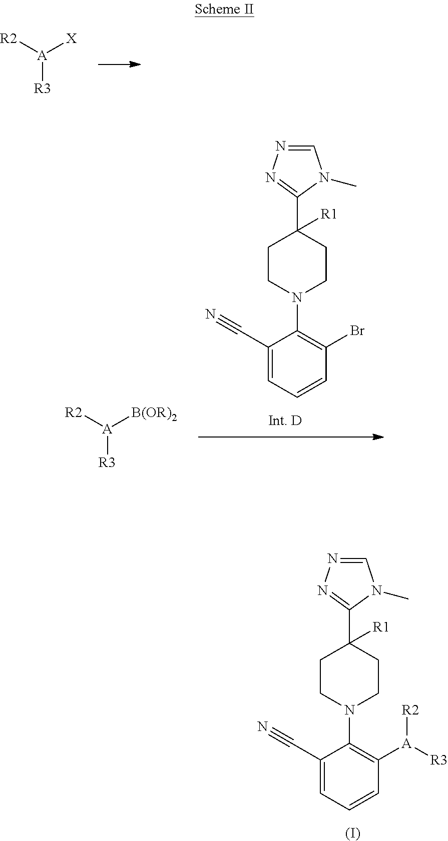

An alternative route to obtain compounds of formula (I) is described in scheme II. The hetero-aryl boronic acid derivative can be prepared from the corresponding halide (R2R3A-X with X being Cl, Br, I) with a suitable borylating agent (e.g. bis(pinacolato)diboron) in the presence of a suitable catalyst (e.g. Pd(dppf)Cl₂*CH₂Cl₂) and a suitable base (e.g. KOAc) at elevated temperatures (e.g. 100° C.). The hetero-aryl boronic acid derivative (R2R3AB(OR)₂) is either isolated as pinacolate boronic acid ester (R═CMe₂ and both R forming a five membered ring together with O, B, O) or boronic acid (R═H) depending on the stability of the boronic acid ester or it can be used in the subsequent Suzuki coupling upon addition of Int. D, a suitable catalyst (e.g. Pd(dppf)Cl₂*CH₂Cl₂) and a suitable base (e.g. aqueous Na₂CO₃ solution). If isolated, the boronic acid derivative can be transformed into examples of general formula I as described in scheme I.

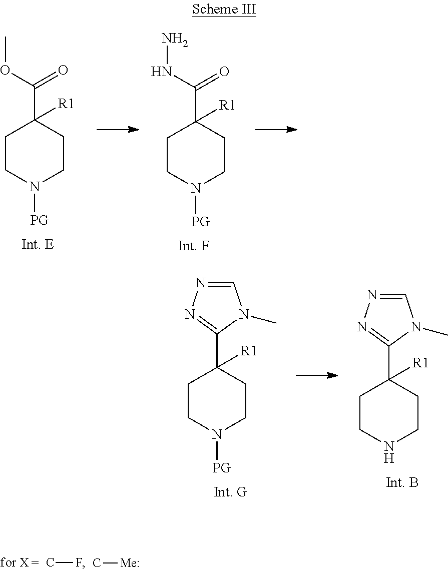

Intermediates Int. B with R1=Me, F can be prepared from the corresponding piperidinyl esters (Int. E) equipped with a suitable protecting group (PG, e.g. BOC) by treatment with a suitable hydrazine source (e.g. N₂H₄*H₂O) at elevated temperature (e.g. 50° C.). The obtained hydrazide (Int. F) is then activated with DMF/DMA at elevated temperature (e.g. 50° C.) and subsequently treated with methyl amine at elevated temperature (e.g. 90° C.) to yield the triazole derivative (Int. G). Intermediates Int. B with R1=Me, F can be obtained by cleaving the protecting group under suitable conditions (e.g. 4N HCl in dioxane for PG=BOC; Pd/C under an H₂ atmosphere for PG=Bn). Intermediate Int. B with R1=H was obtained from commercial sources (CAS No: 297172-18-0).

### EXAMPLES

### Preparation

The compounds according to the invention and their intermediates may be obtained using methods of synthesis which are known to the one skilled in the art and described in the literature of organic synthesis for example using methods described in “Comprehensive Organic Transformations”, 2nd Edition, Richard C. Larock, John Wiley & Sons, 2010, and “March's Advanced Organic Chemistry”, 7th Edition, Michael B. Smith, John Wiley & Sons, 2013. Preferably the compounds are obtained analogously to the methods of preparation explained more fully hereinafter, in particular as described in the experimental section. In some cases the sequence adopted in carrying out the reaction schemes may be varied. Variants of these reactions that are known to the skilled artisan but are not described in detail herein may also be used. The general processes for preparing the compounds according to the invention will become apparent to the skilled man on studying the schemes that follow. Starting compounds are commercially available or may be prepared by methods that are described in the literature or herein, or may be prepared in an analogous or similar manner. Before the reaction is carried out, any corresponding functional groups in the starting compounds may be protected using conventional protecting groups. These protecting groups may be cleaved again at a suitable stage within the reaction sequence using methods familiar to the skilled man and described in the literature for example in “Protecting Groups”, 3rd Edition, Philip J. Kocienski, Thieme, 2005, and “Protective Groups in Organic Synthesis”, 4th Edition, Peter G. M. Wuts, Theodora W. Greene, John Wiley & Sons, 2006. The terms “ambient temperature” and “room temperature” are used interchangeably and designate a temperature of about 20° C., e.g. between 19 and 24° C.

### Abbreviations

| | |
|---|---|
| ACN | acetonitrile |
| Aq. | Aqueous |
| ° C. | Degree celsius |
| CDI | Carbonyl diimidazole |
| CyH/CH | cyclohexane |
| conc. | Concentrated |
| DCM | dichloromethane |
| DIPEA | N,N-diisopropylethylamine |
| DMA | N,N-dimethylacetamide |
| DMF | N,N-dimethylformamide |
| DMSO | dimethyl sulfoxide |
| ESI-MS | Electrospray ionisation mass spectrometry |
| EtOAc | ethyl acetate |
| EtOH | ethanol |
| ex | example |
| eq | equivalent |
| FA | formic acid |
| h | hour |
| HATU | 1-[Bis(dimethylamino)methylene]-1H-1,2,3-triazolo[4,5-b] |
| | pyridinium 3-oxid hexafluorophosphate |
| HCl | Hydrochloric acid |
| HPLC | High performance liquid chromatography |
| Int. | Intermediate |
| K₂CO₃ | potassium carbonate |
| K(OtBu) | Potassium tert. butoxide |
| L | liter |
| LiOH*H₂O | Lithium hydroxide monohydrate |
| M | molar |
| MeOH | methanol |
| MgSO₄ | magnesium sulphate |
| min | minute |
| mL | milliliter |
| MTBE | tert-butylmethylether |
| NaOEt | Sodium ethanolate |
| NH₃ | ammonia |
| PMB | Para-methoxy benzyl |
| Prep. | preparative |
| RF | Reversed phase |
| RT | room temperature (about 20° C.) |
| sat. | saturated |
| TBTU | Benzotriazolyl tetramethyluronium tetrafluoroborate |
| TEA | triethylamine |
| TFA | trifluoroacetic acid |
| TFAA | trifluoroacetic anhydride |
| THF | Tetrahydrofuran |
| TMS-Cl | Trimethylsilyl chloride |

### Preparation of Intermediates

### Intermediate I

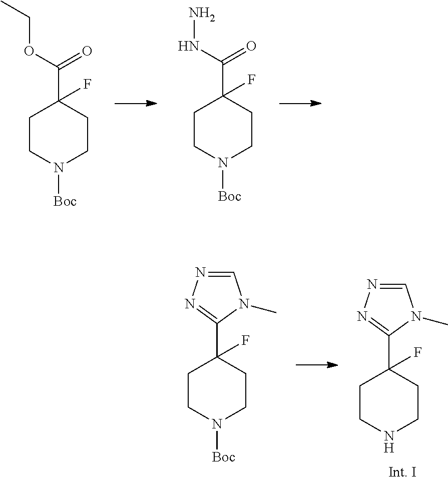

### tert-Butyl 4-fluoro-4-(hydrazinecarbonyl)piperidine-1-carboxylate

1-tert-Butyl 4-ethyl 4-fluoropiperidine-1,4-dicarboxylate (160 g, 0.58 mol) is suspended in ethanol (640 mL) in a round-bottom flask. Hydrazine hydrate (70.6 mL, 1.16 mol) is added to the mixture at ambient temperature. The reaction mixture is heated to 50° C. and stirred for 12 h. After cooling to ambient temperature, the mixture is concentrated under reduced pressure to yield tert-butyl 4-fluoro-4-(hydrazinecarbonyl)piperidine-1-carboxylate in 80% purity.

C₁₁H₂₀FN₃O₃ (M=261.3 g/mol)

ESI-MS: 284.2 [M+Na]+

Rt (HPLC): 0.615 min (method A)

### tert-Butyl 4-fluoro-4-(4-methyl-4H-1,2,4-triazol-3-yl)piperidine-1-carboxylate

tert-Butyl 4-fluoro-4-(hydrazinecarbonyl)piperidine-1-carboxylate (135 g, 0.413 mol, 80% purity) is mixed with dioxane (945 mL) in a round-bottom-flask. N,N-Dimethylformamid-dimethylacetal (137 mL, 1.03 mol) is added to the mixture at ambient temperature. The reaction mixture is heated to 50° C. and stirred for 1 h. A solution of methylamine (299 g, 30% in EtOH, 2.89 mol) and acetic acid (165 mL, 2.89 mol) are added into the mixture. The resulting reaction mixture is heated to 90° C. and stirred for 11 h. The mixture is concentrated under reduced pressure. The residue is purified by column chromatography (SiO₂, PE/EtOAc gradient 20:1 to 0:1) to obtain tert-butyl 4-fluoro-4-(4-methyl-4H-1,2,4-triazol-3-yl)piperidine-1-carboxylate.

C₁₃H₂₁FN₄O₂ (M=284.3 g/mol)

ESI-MS: 285.1 [M+H]+

Rt (HPLC): 0.766 min (method A)

### Intermediate I: 4-fluoro-4-(4-methyl-4H-1,2,4-triazol-3-yl)piperidine

tert-Butyl 4-fluoro-4-(4-methyl-4H-1,2,4-triazol-3-yl)piperidine-1-carboxylate (90 g, 0.316 mol) is combined with MeOH (90 mL) in a round-bottom flask. A solution of HCl (4 M in MeOH, 450 mL, 1.79 mol) is added slowly at ambient temperature. The resulting reaction mixture is stirred at ambient temperature for 12 h. The desired product is collected by filtration, washed with MeOH and dried to yield 4-fluoro-4-(4-methyl-4H-1,2,4-triazol-3-yl)piperidine hydrochloride salt.

The hydrochloride salt (13.5 g) is added to a solution of ammonia in MeOH (7 N, 150 mL) and purified by chromatography (Biotage SNAP Cartridge KP-NH, gradient DCM/MeOH 4:1 to 7:3)
C₈H₁₃FN₄ (M=184.2 g/mol)

ESI-MS: 185 [M+H]+

Rt (HPLC): 0.20 min (method B)

4-(4-Methyl-4H-1,2,4-triazol-3-yl)piperidine (MFCD09055373, CAS: 297172-18-0), 4-methyl-4-(4-methyl-4H-1,2,4-triazol-3-yl)piperidine dihydrochloride (MFCD32875324) were obtained from commercial vendors. Hydrochloride salts were converted into the free piperidine or piperazine according to the procedure described for Int. I

### Intermediate 11.1

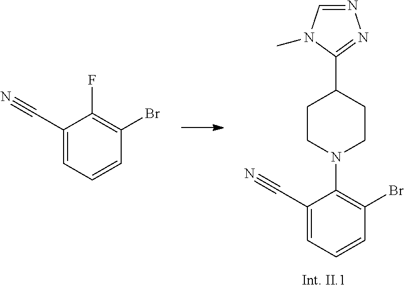

A solution of 3-bromo-2-fluorobenzonitrile (1.5 g, 7.5 mmol) and 4-(4-methyl-4H-1,2,4-triazol-3-yl)piperidine (3.6 g, 22 mmol) in DMSO (18 mL) is stirred at 130° C. for 18 h. The reaction mixture is diluted with ACN and directly subjected to purification via preparative HPLC (Xbridge C18, ACN/water gradient containing 0.1% TFA) to give intermediate II.1
C₁₅H₁₆BrN₅ (M=346.2 g/mol)

ESI-MS: 346/348 [M+H]+

Rt (HPLC): 0.40 min (method C)

| | | | | Molecular Formula (MW) |
|---|---|---|---|---|
| | | | Deviation from | ESI-MS |
| | Starting | | general | HPLC retention time |
| Int. | material | Structure | procedure | (method) |
| II.2 | Int. I + 3-bromo-2- fluoro-benzo- nitrile | 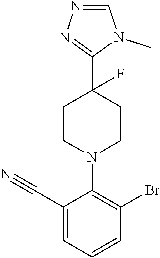 [SMILES: Cn1cnnc1C1(F)CCN(c2c(Br)cccc2C#N)CC1] | 17 h at 100° C. | C₁₅H₁₅BrFN₅ (M = 364.2 g/mol) ESI-MS: 364/366 [M + H]+ Rt (HPLC): 0.49 min (method C) |
| II.3 | 4-methyl-4-(4- methyl-1,2,4- triazol-3-yl) piperidine + 3-bromo-2- fluoro-benzo- nitrile | 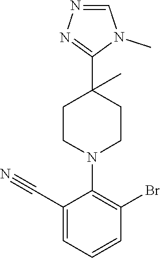 [SMILES: Cn1cnnc1C1(C)CCN(c2c(Br)cccc2C#N)CC1] | 20 h at 120° C.; purified by preparative HPLC (Sunfire C18; MeCN/water gradient con- taining 0.1% TFA) | C₁₆H₁₈BrN₅ (M = 360.3 g/mol) ESI-MS: 360/362 [M + H]+ Rt (HPLC): 0.74 min (method D) |

### Intermediate III.1

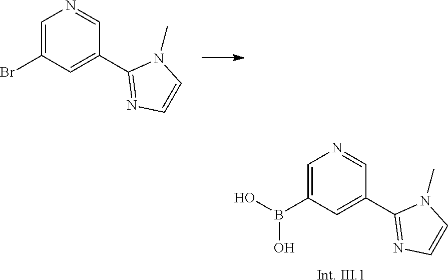

To a stirred solution of 3-bromo-5-(1-methyl-1-H-imidazol-2-yl)pyridine (50 mg, 0.20 mmol), bis(pinacolato)diboron (63 mg, 0.25 mmol), and potassium acetate (39 mg, 0.40 mmol) in 1,4-dioxane (1 mL) is added [1,1′-Bis-(diphenylphosphino)-ferrocen]-dichloropalladium(II) (Pd(dppf)Cl₂, CAS: 72287-26-4) (7.2 mg, 0.01 mmol). After being stirred at 100° C. for 1.5 h and then at ambient temperature for 18 h, the mixture is diluted with DCM and H₂O. The organic phase is separated and concentrated to give crude Int. III.1, which is used in the subsequent step without further purification.

C₁₅H₂₀BN₃O₂ (M=203.0 g/mol)

ESI-MS: 204 [M+H]+

Rt (HPLC): 0.20 min (method C)

### Intermediate III.2

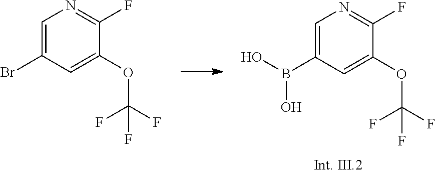 [SMILES: Fc1ncc(Br)cc1OC(F)(F)F ; OB(O)c1cnc(F)c(OC(F)(F)F)c1 ; "Int. III.2"]

To a stirred solution of 5-bromo-2-fluoro-3-(trifluoromethoxy)pyridine (CAS: 1361822-98-1) (50 mg, 0.16 mmol), bis(pinacolato)diboron (50 mg, 0.20 mmol), and potassium acetate (39 mg, 0.40 mmol) in 1,4-dioxane (1 mL) is added [1,1′-Bis-(diphenylphosphino)-ferrocen]-dichloro-palladium(II) (Pd(dppf)Cl₂, CAS: 72287-26-4) (7.2 mg, 0.01 mmol). After being stirred at 100° C. for 45 min, the mixture is diluted with DCM and H₂O. The organic phase is separated and concentrated to give crude Int. III.2, which is used in subsequent step without further purification.

C₆H₄BF₄NO₃ (M=224.9 g/mol)

ESI-MS: 225/226 [M+H]+

Rt (HPLC): 0.45 min (method C)

### Synthesis of Intermediate III.3a and III.3b

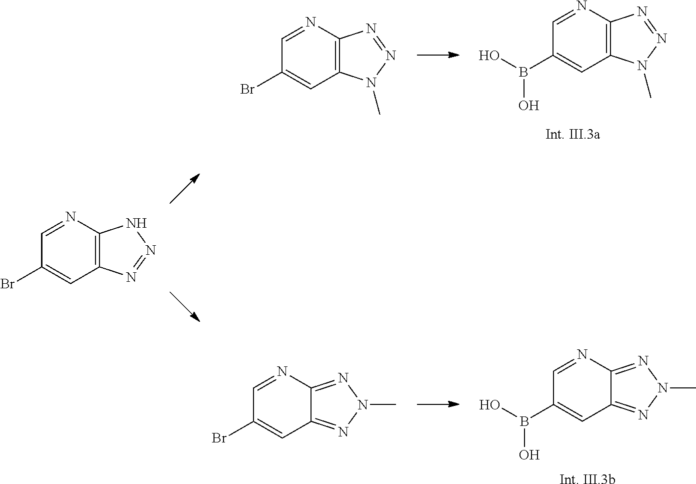

### 6-Bromo-1-methyl-1H-[1,2,3]triazolo[4,5-b]pyridine and 6-bromo-2-methyl-2H[1,2,3]triazolo[4,5-b]pyridine

To a stirred solution of 6-bromo-3H-[1,2,3]triazolo[4,5-b]pyridine (CAS: 92276-38-5) (0.550 g, 2.71 mmol) in DMSO (5 mL) are added DIPEA (0.92 mL, 5.4 mmol) and iodomethane (0.17 mL, 2.7 mmol). After being stirred at 90° C. for 18 h, the reaction mixture is cooled to ambient temperature and directly subjected to purification via preparative HPLC (Xbridge C18, MeCN/water gradient containing 0.1% NH₃) to give the corresponding methylation regioisomers.

### 6-Bromo-1-methyl-1H-[1,2,3]triazolo[4,5-b]pyridine

C₆H₅BrN₄ (M=213.0 g/mol)

ESI-MS: 213/215 [M+H]+

Rt (HPLC): 0.36 min (method B)

### 6-Bromo-2-methyl-2H-[1,2,3]triazolo[4,5-b]pyridine (co-eluted with regioisomer 6-bromo-3-methyl-3H-[1,2,3]triazolo[4,5-b]pyridine)

C₆H₅BrN₄ (M=213.0 g/mol)

ESI-MS: 213/215 [M+H]+

Rt (HPLC): 0.44 min (method B)

### Intermediate III.3a

To a stirred solution of 6-bromo-1-methyl-1H-[1,2,3]triazolo[4,5-b]pyridine (171 mg, 0.80 mmol) in 1,4-dioxane (2 mL) are added bis(pinacolato)diboron (305 mg, 1.20 mmol) and potassium acetate (236 mg, 2.41 mmol). The resulting mixture is purged with Ar for 10 min, followed by addition of bis(triphenylphosphine)palladium chloride (CAS: 13965-03-2) (56 mg, 0.08 mmol). After being stirred at 90° C. for 5 h, the mixture is cooled to ambient temperature, concentrated, redissolved in MeCN and H₂O, and subjected to purification via preparative HPLC (Xbridge C18, MeCN/water gradient containing 0.1% TFA) to give intermediate III.3a.

C₆H₇BN₄O₂ (M=178.0 g/mol)

ESI-MS: 179 [M+H]+

Rt (HPLC): 0.21 min (method C)

### Intermediate III.3b

To a stirred solution of 1:1 mixture of 6-bromo-2-methyl-2H-[1,2,3]triazolo[4,5-b]pyridine and 6-bromo-3-methyl-3H-[1,2,3]triazolo[4,5-b]pyridine (310 mg, 0.73 mmol) in 1,4-dioxane (2 mL) are added bis(pinacolato)diboron (276 mg, 1.09 mmol) and potassium acetate (214 mg, 2.18 mmol). The resulting mixture is purged with Ar for 10 min, followed by addition of bis(triphenylphosphine)palladium chloride (CAS: 13965-03-2) (51 mg, 0.07 mmol). After being stirred at 100° C. for 3 h, the mixture is cooled to ambient temperature, diluted with EtOAc, and filtered through a silica plug. Filtrate is concentrated, redissolved in MeCN and H₂O, and subjected to purification via preparative HPLC (Xbridge C18, MeCN/water gradient containing 0.1% TFA) to give 1:1 mixture of intermediate III.3b and the corresponding regioisomeric boronic acid, which was used without further purification.

C₆H₇BN₄O₂ (M=178.0 g/mol)

ESI-MS: 179 [M+H]+

Rt (HPLC): 0.21 min (method C)

### Synthesis of Intermediate III.4

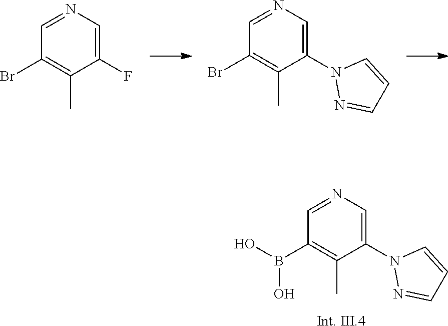

### 3-Bromo-4-methyl-5-(1H-pyrazol-1-yl)pyridine

A solution of 3-bromo-5-fluoro-4-methylpyridine (0.25 mL, 1.2 mmol), pyrazole (87 mg, 1.2 mmol), and Cs₂CO₃ (1.22 g, 3.75 mmol) in DMSO (2 mL) is stirred at 90° C. for 18 h. After being cooled to ambient temperature, the mixture is diluted with MeCN/H₂O and directly purified via preparative HPLC (Xbridge C18, MeCN/water gradient containing 0.1% NH₃) to give 3-bromo-4-methyl-5-(1H-pyrazol-1-yl)pyridine.

C₉H₈BrN₃ (M=238.1 g/mol)

ESI-MS: 238/240 [M+H]+

Rt (HPLC): 0.52 min (method C)

### Intermediate III.4

To a stirred solution of 3-bromo-4-methyl-5-(1H-pyrazol-1-yl)pyridine (130 mg, 0.55 mmol) in 1,4-dioxane (1 mL) are added bis(pinacolato)diboron (207 mg, 0.82 mmol) and potassium acetate (160 mg, 1.64 mmol). The resulting mixture is purged with Ar for 10 min, followed by addition of bis(triphenylphosphine)palladium chloride (CAS: 13965-03-2) (38 mg, 0.05 mmol). After being stirred at 100° C. for 3 h, the mixture is cooled to ambient temperature, diluted with EtOAc, and filtered through a silica plug. Filtrate is concentrated, redissolved in MeCN and H2O, and subjected to purification via preparative HPLC (Xbridge C18, MeCN/water gradient containing 0.1% TFA) to give Int. III.4.

C₉H₁₀BN₃O₂ (M=203.0 g/mol)

ESI-MS: 204 [M+H]+

Rt (HPLC): 0.22 min (method C)

### Synthesis of Intermediate III.5

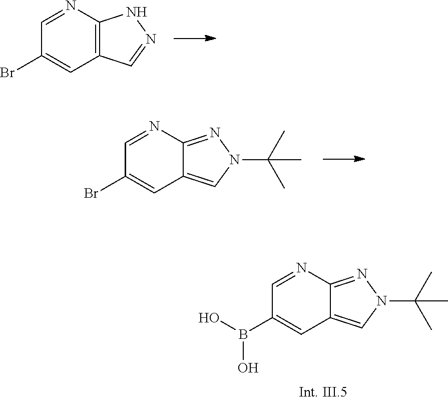 [SMILES: Brc1cnc2[nH]ncc2c1 ; CC(C)(C)n1cc2cc(Br)cnc2n1 ; CC(C)(C)n1cc2cc(B(O)O)cnc2n1 ; "Int. III.5"]

### 5-Bromo-2-tert-butyl-2H-pyrazolo[3,4-b]pyridine

To a stirred solution of 5-bromo-1H-pyrazolo[3,4-b]pyridine (4.00 g, 19.8 mmol) in toluene (23 mL) are added tert-butyl acetate (26.6 mL, 198 mmol) and methanesulfonic acid (1.3 mL, 19.8 mmol). After being stirred at 80° C. for 1 h, the reaction is treated with additional methanesulfonic acid (1.3 mL, 19.8 mmol). The reaction mixture is cooled to ambient temperature, concentrated, redissolved in MeCN/H₂O, and purified via preparative HPLC (Xbridge C18, ACN/water gradient containing 0.1% TFA) to give 5-bromo-2-tertbutyl-2H-pyrazolo[3,4-b]pyridine.

C₁₀H₁₂BrN₃ (M=254.1 g/mol)

ESI-MS: 254/256 [M+H]+

Rt (HPLC): 0.50 min (method C)

### Intermediate III.5

A solution of 5-bromo-2-tert-butyl-2H-pyrazolo[3,4-b]pyridine (1.50 g, 3.87 mmol), bis(pinacolato)diboron (1.20 g, 4.78 mmol), and potassium acetate (763 mg, 7.77 mmol) in 1,4-dioxane (15 mL) is purged with Ar for 10 min, followed by addition of [1,1′-bis-(diphenylphosphino)-ferrocen]-dichloro-palladium(II) (Pd(dppf)Cl₂, CAS: 72287-26-4) (190 mg, 0.23 mmol). After being stirred at 110° C. for 4 h, the mixture is cooled to ambient temperature, concentrated, redissolved in MeCN/H₂O, and purified via preparative HPLC (Xbridge C18, ACN/water gradient containing 0.1% TFA) to give Int. III.5.

C₁₀H₁₄BN₃O₂ (M=219.0 g/mol)

ESI-MS: 220 [M+H]+

Rt (HPLC): 0.27 min (method C)

### Synthesis of Intermediate III.6

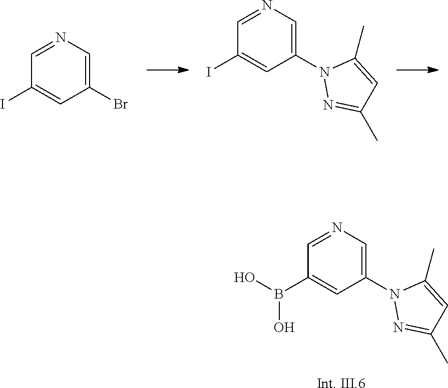 [SMILES: Brc1cncc(I)c1 ; Cc1cc(C)n(-c2cncc(I)c2)n1 ; Cc1cc(C)n(-c2cncc(B(O)O)c2)n1 ; "Int. III.6"]

### 3-(3,5-Dimethyl-1H-pyrazol-1-yl)-5-iodopyridine

A solution of 3-bromo-5-iodopyridine (591 mg, 2.08 mmol), 3,5-dimethyl-1H-pyrazole (100 mg, 1.04 mmol), DL-proline (12 mg, 0.10 mmol), CuI (20 mg, 0.10 mmol), Cs₂CO₃ (339 mg, 1.04 mmol) in DMF (1 mL) is purged with Ar for 10 min. After being stirred at 120° C. for 18 h, the mixture is cooled to ambient temperature, diluted with MeOH, and directly purified via preparative HPLC (Xbridge C18, ACN/water gradient containing 0.1% TFA) to give 3-(3,5-dimethyl-1H-pyrazol-1-yl)-5-iodopyridine.

C₁₀H₁₀IN₃ (M=299.1 g/mol)

ESI-MS: 300 [M+H]+

Rt (HPLC): 0.53 min (method C)

### Intermediate III.6

To a stirred solution of 3-(3,5-dimethyl-1H-pyrazol-1-yl)-5-iodopyridine (50 mg, 0.17 mmol), bis(pinacolato)diboron (52 mg, 0.21 mmol), and potassium acetate (33 mg, 0.34 mmol) in 1,4-dioxane (1 mL) is added [1,1′-Bis-(diphenylphosphino)-ferrocen]-dichloropalladium(II) (Pd(dppf)Cl₂, CAS: 72287-26-4) (8.0 mg, 0.01 mmol). After being stirred at 100° C. for 18 h, the mixture is diluted with DCM and H₂O. Organic phase is concentrated to give crude Int. III.6, which is used in subsequent step without further purification.

C₁₀H₁₂BN₃O₂ (M=217.0 g/mol)

ESI-MS: 218 [M+H]+

Rt (HPLC): 0.28 min (method C)

### Synthesis of Intermediate III.7

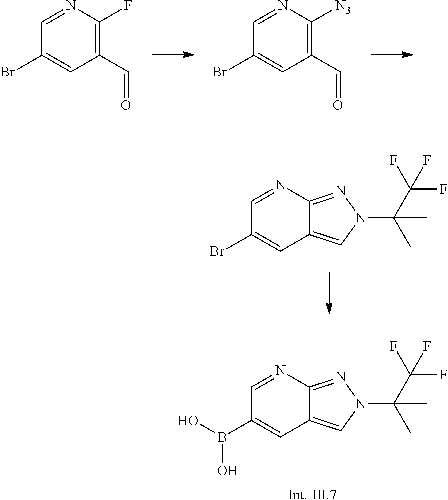 [SMILES: O=Cc1cc(Br)cnc1F ; [N-]=[N+]=Nc1ncc(Br)cc1C=O ; CC(C)(n1cc2cc(Br)cnc2n1)C(F)(F)F ; CC(C)(n1cc2cc(B(O)O)cnc2n1)C(F)(F)F ; "Int. III.7"]

### 2-Azido-5-bromopyridine-3-carbaldehyde

To a stirred solution of 5-bromo-2-fluoropyridine-3-carbaldehyde (1.0 g, 4.7 mmol) and tetrabutylammonium iodide (172 mg, 0.47 mmol) in DMSO (6 mL) is added sodium azide (367 mg, 5.6 mmol). After being stirred for 45 min, the reaction is diluted with H₂O, and precipitate is collected via filtration and dried to give 2-azido-5-bromopyridine-3-carbaldehyde, which is used in subsequent step without further purification.

C₆H₃BrN₄₀ (M=227.0 g/mol)

ESI-MS: 227/229 [M+H]+

Rt (HPLC): 0.25 min (method C)

### 5-Bromo-2-(1,1,1-trifluoro-2-methylpropan-2-yl)-2H-pyrazolo[3,4-b]pyridine

To a stirred solution of 2-azido-5-bromopyridine-3-carbaldehyde (310 mg, 1.37 mmol) and 1,1,1-trifluoro-2-methylpropan-2-amine hydrochloride (335 mg, 2.05 mmol) in ethanol (6 ml) is added 3A molecular sieves. After being stirred for 18 h, the mixture is concentrated, redissolved in tolune (6 mL), and further stirred for 18 h. The resulting mixture is concentrated, redissolved in MeCN/H₂O, and purified via preparative HPLC (Xbridge C18, MeCN/water gradient containing 0.1% NH₃) to give 5-bromo-2-(1,1,1-trifluoro-2-methylpropan-2-yl)-2H-pyrazolo[3,4-b]pyridine.

C₁₀H₉BrF₃N₃ (M=308.1 g/mol)

ESI-MS: 308/310 [M+H]+

Rt (HPLC): 0.62 min (method B)

### Intermediate III.7

A solution of 5-bromo-2-(1,1,1-trifluoro-2-methylpropan-2-yl)-2H-pyrazolo[3,4-b]pyridine (643 mg, 1.67 mmol), bis(pinacolato)diboron (678 mg, 2.67 mmol), and potassium acetate (639 mg, 6.51 mmol) in 1,4-dioxane (6 mL) is purged with Ar for 15 min, followed by addition of bis(triphenylphosphine)palladium chloride (CAS: 13965-03-2) (141 mg, 0.20 mmol). After being stirred at 60° C. for 10 h, the mixture is cooled to ambient temperature, concentrated, redissolved in MeCN/H₂O, and purified via preparative HPLC (X-bridge C18, MeCN/water gradient containing 0.1% TFA) to give Int. III.7.

C₁₀H₁₁BF₃N₃O₂ (M=273.0 g/mol)

ESI-MS: 274 [M+H]+

Rt (HPLC): 0.32 min (method C)

### Synthesis of Intermediate III.8a and III.8b

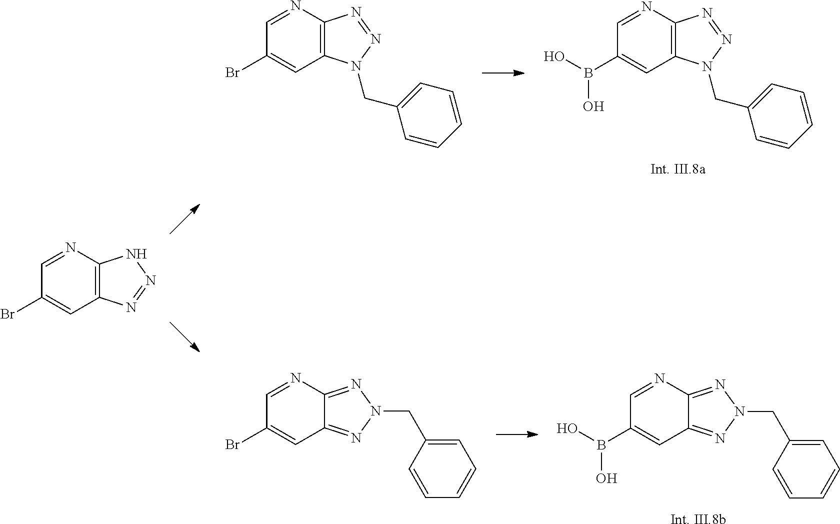

### 1-Benzyl-6-bromo-1H-[1,2,3]triazolo[4,5-b]pyridine and 2-benzyl-6-bromo-2H[1,2,3]triazolo[4,5-b]pyridine

To a stirred solution of 6-bromo-3H-[1,2,3]triazolo[l4,5-b]pyridine (0.600 g, 2.95 mmol) in DMSO (5 mL) are added DIPEA (1.0 mL, 5.9 mmol) and benzyl bromide (0.36 mL, 2.9 mmol). After being stirred at 90° C. for 18 h, the reaction mixture is cooled to ambient temperature and directly subjected to purification via preparative HPLC (Xbridge C18, MeCN/water gradient containing 0.1% NH₃) to give the corresponding benzylation regioisomers.

### 1-Benzyl-6-bromo-1H-[1,2,3]triazolo[4,5-b]pyridine

C₁₂H₉BrN₄ (M=289.1 g/mol)

ESI-MS: 289/291 [M+H]+

Rt (HPLC): 0.52 min (method C)

### 2-Benzyl-6-bromo-2H-[1,2,3]triazolo[4,5-b]pyridine (co-eluted with regioisomer 3-benzyl-6-bromo-3H-[1,2,3]triazolo[4,5-b]pyridine)

C₁₂H₉BrN₄ (M=289.1 g/mol)

ESI-MS: 289/291 [M+H]+

Rt (HPLC): 0.59 min (method C)

### Intermediate III.8a

To a stirred solution of 1-benzyl-6-bromo-1H-[1,2,3]triazolo[4,5-b]pyridine (386 mg, 1.34 mmol) in 1,4-dioxane (2 mL) are added bis(pinacolato)diboron (507 mg, 2.00 mmol) and potassium acetate (392 mg, 4.01 mmol). The resulting mixture is purged with Ar for 10 min, followed by addition of bis(triphenylphosphine)palladium chloride (CAS: 13965-03-2) (94 mg, 0.13 mmol). After being stirred at 90° C. for 5 h, the mixture is cooled to ambient temperature, concentrated, redissolved in MeCN and H₂O, and subjected to purification via preparative HPLC (Xbridge C18, MeCN/water gradient containing 0.1% TFA) to give intermediate III.8a.

C₁₂H₁₁BN₄O₂ (M=254.1 g/mol)

ESI-MS: 255 [M+H]+

Rt (HPLC): 0.38 min (method C)

### Intermediate III.8b

To a stirred solution of 1:1 mixture of 2-benzyl-6-bromo-2H-[1,2,3]triazolo[4,5-b]pyridine and 3-benzyl-6-bromo-3H-[1,2,3]triazolo[4,5-b]pyridine (310 mg, 1.07 mmol) in 1,4-dioxane (2 mL) are added bis(pinacolato)diboron (407 mg, 1.61 mmol) and potassium acetate (315 mg, 3.22 mmol). The resulting mixture is purged with Ar for 10 min, followed by addition of bis(triphenylphosphine)palladium chloride (CAS: 13965-03-2) (75 mg, 0.11 mmol). After being stirred at 100° C. for 3 h, the mixture is cooled to ambient temperature, diluted with EtOAc, and filtered through a silica plug. Filtrate is concentrated, redissolved in MeCN and H₂O, and subjected to purification via preparative HPLC (Xbridge C18, MeCN/water gradient containing 0.1% TFA) to give intermediate III.8b.

C₁₂H₁₁BN₄O₂ (M=254.1 g/mol)

ESI-MS: 255 [M+H]+

Rt (HPLC): 0.41 min (method C)

### Intermediate III.9

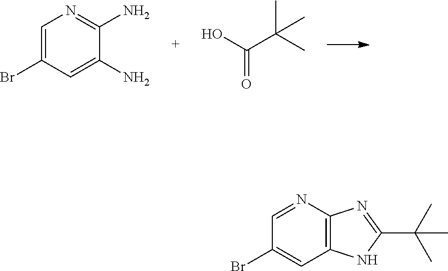

A mixture of 2,3-diamino-5-bromopyridine (200 mg, 1.01 mmol) and pivalic acid (2.09 g, 20.2 mmol) is heated neat to 120° C. under vigorous stirring for 10 h, to 140° C. for 10 h and to 150° C. for 20 h. After cooling to ambient temperature, the mixture is diluted with EtOAc and the resulting solution washed three times with aqueous K₂CO₃ solution (2 M). The organic phase is dried over Na₂SO₄ and concentrated. The residue is purified by preparative HPLC (XBridge C18, ACN/water gradient containing 0.1% NH₃) to yield the desired product.

C₁₀H₁₂BrN₃ (M=254.1 g/mol)

ESI-MS: 254/256 [M+H]+

Rt (HPLC): 0.62 min (method D)

### Synthesis of Intermediate III.10

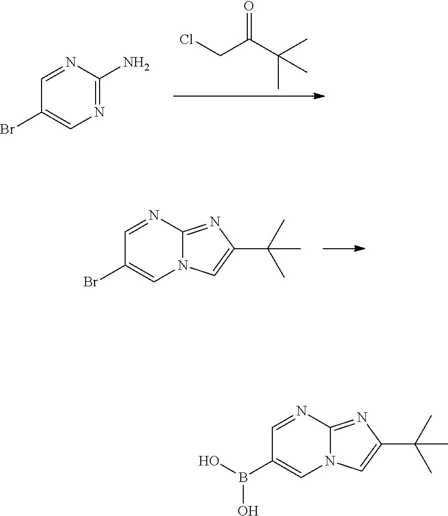

### 6-bromo-2-tert-butylimidazo[1,2-a]pyrimidine

Ethanol (2 mL) is added to a mixture of 2-amino-5-brompyrimidine (1.00 g, 5.63 mmol) and 1-chloropinacolone (1.14 mL, 8.5 mmol). The resulting mixture is stirred at 90° C. for 5 days. After cooling to ambient temperature, the mixture is loaded onto EXtrelut® and purified by column chromatography (SiO₂, DCM/MeOH gradient) to yield the desired product.

C₁₀H₁₂BrN₃ (M=254.1 g/mol)

ESI-MS: 254/256 [M+H]+

Rt (HPLC): 0.28 min (method C)

### Intermediate III.10

6-Bromo-2-tert-butylimidazo[1,2-a]pyrimidine (144 mg, 0.567 mmol) is added to 1,4-dioxane (1.0 mL). Bis(pinacolato)diborane (215.0 mg, 850 mmol) and potassium acetate (167 mg, 1.70 mmol) is added and the resulting mixture is degassed by passing an Argon flow through the mixture. Pd(PPh₃)₂Cl₂ (39.8 mg, 0.057 mmol) is added and the reaction mixture is heated to 90° C. and stirred for 5 h. After cooling to ambient temperature, the mixture is concentrated, resuspended in a mixture of water and ACN and purified by preparative HPLC (XBridge C18, ACN/water gradient containing 0.1% TFA) to yield the desired product.

C₁₀H₁₄BN₃O₂ (M=219.1 g/mol)

ESI-MS: 220 [M+H]+

Rt (HPLC): 0.25 min (method C)

### Synthesis of Intermediate III.11

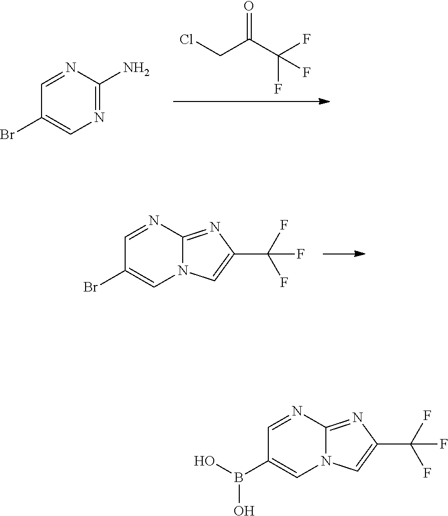 [SMILES: Nc1ncc(Br)cn1 ; O=C(CCl)C(F)(F)F ; FC(F)(F)c1cn2cc(Br)cnc2n1 ; OB(O)c1cnc2nc(C(F)(F)F)cn2c1]

### 6-bromo-2-trifluoromethylimidazo[1,2-a]pyrimidine

Ethanol (2 mL) is added to a mixture of 2-amino-5-brompyrimidine (1.00 g, 5.63 mmol) and 1-chloro-3,3,3-trifluoroacetone (889 μL, 8.45 mmol). The resulting mixture is stirred at 90° C. for 5 days. After cooling to ambient temperature, the mixture is loaded onto EXtrelut® and purified by column chromatography (SiO₂, DCM/MeOH gradient) to yield the desired product.

C₇H₃BrF₃N₃ (M=266.1 g/mol)

ESI-MS: 266/268 [M+H]+

Rt (HPLC): 0.38 min (method C)

### Intermediate III.11

6-Bromo-2-trifluoromethylimidazo[1,2-a]pyrimidine (82 mg, 0.308 mmol) is added to 1,4-dioxane (1.0 mL). Bis(pinacolato)diborane (117 mg, 462 mmol) and potassium acetate (90.6 mg, 0.925 mmol) is added and the resulting mixture is degassed by passing an Argon flow through the mixture. Pd(PPh₃)₂Cl₂ (21.6 mg, 0.031 mmol) is added and the reaction mixture is heated to 90° C. and stirred for 5 h. After cooling to ambient temperature, the mixture is concentrated, resuspended in a mixture of water and ACN and purified by preparative HPLC (XBridge C₁₈, ACN/water gradient containing 0.1% TFA) to yield the desired product.

C₇H₅BF₃N₃O₂ (M=230.9 g/mol)

ESI-MS: 232 [M+H]+

Rt (HPLC): 0.29 min (method C)

### Synthesis of Intermediate III.12

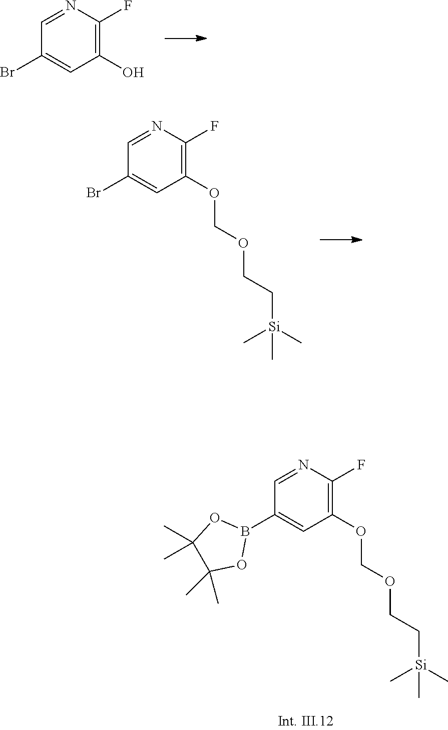

### 5-Bromo-2-fluoro-3-{[2-(trimethylsilyl)ethoxy]methoxy}pyridine

5-Bromo-2-fluoro-pyridin-3-ol (4.00 g, 20.4 mmol) and diisopropylethylamine (5.28 g, 40.8 mmol) are added to DCM (100 mL) and 2-(trimethylsilyl)ethoxymethyl chloride (3.94 g, 22.5 mmol) is added dropwise via syringe under stirring. The resulting reaction mixture is stirred at ambient temperature for 90 minutes. The mixture is concentrated and diluted with EtOAc and water. The organic phase is separated, dried over Na₂SO₄ and concentrated. The residue is purified by column chromatography (SiO₂, CyH/EtOAc gradient) to yield the desired product.

C₁₁H₁₇BrFNO₂Si (M=322.2 g/mol)

ESI-MS: 322/324 [M+H]+

Rt (HPLC): 0.90 min (method C)

### Intermediate III.12

5-Bromo-2-fluoro-3-{[2-(trimethylsilyl)ethoxy]methoxy}pyridine (3.0 g, 9.31 mmol), bis(pinacolato)diboron (4.68 g, 18.4 mmol) and potassium acetate (2.74 g, 27.9 mmol) are suspended in 1,4-dioxane (30 mL) and the resulting mixture is purged with argon for 15 min. [1,1′-Bis-(diphenylphosphino)-ferrocen]-dichloro-palladium(II) (Pd(dppf)Cl₂; CAS: 72287-26-4) (476 mg, 0.652 mmol) is added, and the mixture is further purged with argon for 3 min. The reaction mixture is heated to 6 h at 80° C. After cooling to ambient temperature, the mixture is diluted with EtOAc and water. The organic phase is separated and dried over Na₂SO₄. Charcoal is added, the mixture is filtered through celite and concentrated to yield the desired product, which was used in the next step without further purification.

C₁₇H₂₉BFNO₄Si (M=369.3 g/mol)

ESI-MS: 370 [M+H]+

Rt (HPLC): 0.90 min (method C)

### Synthesis of Intermediate III.13

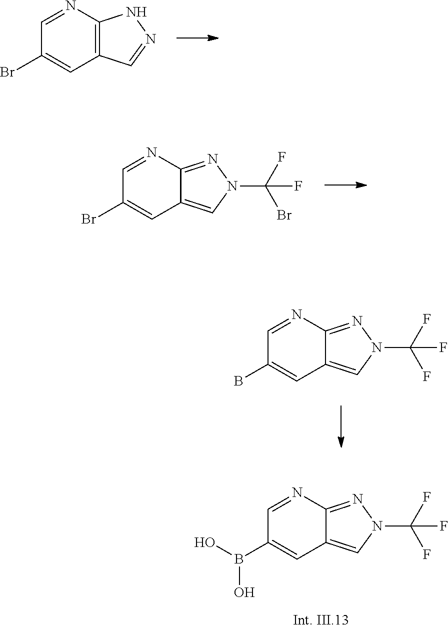

### 5-Bromo-2-(bromodifluoromethyl)-2H-pyrazolo[3,4-b]pyridine

To a stirred solution of 5-bromo-1H-pyrazolo[3,4-b]pyridine (6.00 g, 28.8 mmol) in DMF (200 mL) is added sodium hydride (1.50 g, 34.5 mmol; 55% in mineral oil) at 0° C. After being stirred for 30 min, the reaction mixture is treated with dibromodifluoromethane (8.3 mL, 86.3 mmol) and warmed to ambient temperature. The resulting mixture is stirred for 18 h, diluted with MeCN/H2O, and directly purified via preparative HPLC (Xbridge C18, MeCN/water gradient containing 0.1% TFA) to give 5-bromo-2-(bromodifluoromethyl)₂H-pyrazolo[3,4-b]pyridine.

C₇H₃Br₂F₂N₃ (M=326.9 g/mol)

ESI-MS: 326/328/330 [M+H]+

Rt (HPLC): 0.56 min (method C)

### 5-Bromo-2-(trifluoromethyl)-2H-pyrazolo[3,4-b]pyridine

A solution of 5-bromo-2-(bromodifluoromethyl)-2H-pyrazolo[3,4-b]pyridine (2.1 g, 6.4 mmol) and silver tetrafluoroborate (2.5 g, 12 mmol) in DCM (40 mL) is stirred at 50° C. for 18 h. The reaction mixture is concentrated, redissolved in DCE (40 mL), and stirred at 80° C. for 18 h. The resulting mixture is concentrated, loaded onto EXtrelut®, and purified by column chromatography (SiO₂, DCM/MeOH gradient 100/0 to 1/1) to yield the title compound.

C₇H₃BrF₃N₃ (M=266.0 g/mol)

ESI-MS: 266/268 [M+H]+

Rt (HPLC): 0.47 min (method C)

### Intermediate III.13

To a stirred solution of 5-bromo-2-(trifluoromethyl)-2H-pyrazolo[3,4-b]pyridine (741 mg, 1.39 mmol) in 1,4-dioxane (10 mL) are added bis(pinacolato)diboron (529 mg, 2.09 mmol) and potassium acetate (409 mg, 4.18 mmol). The resulting mixture is purged with Ar for 10 min, followed by addition of [1,1′-bis-(diphenylphosphino)-ferrocen]-dichloro-palladium(II) (Pd(dppf)Cl₂; CAS: 72287-26-4) (102 mg, 0.14 mmol). After being stirred at 90° C. for 5 h, the mixture is cooled to ambient temperature, concentrated, redissolved in H₂O/MeCN, and purified via preparative HPLC (Xbridge C18, MeCN/water gradient containing 0.1% TFA) to give Int. III.13.

C₇H₅BF₃N₃O₂ (M=230.9 g/mol)

ESI-MS: 232 [M+H]+

Rt (HPLC): 0.30 min (method C)

### Synthesis of Intermediate III.14

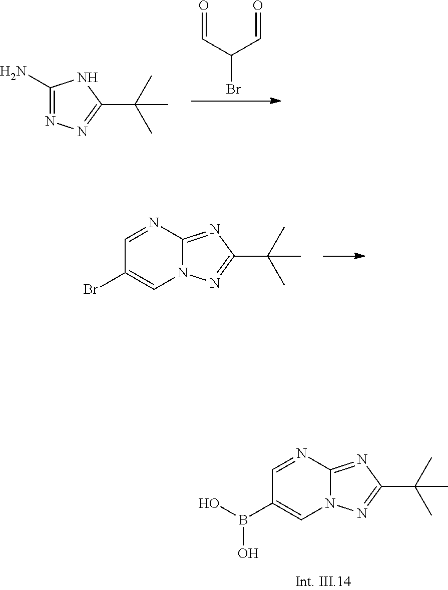

### 6-Bromo-2-tert-butyl-[1,2,4]triazolo[1,5-a]pyrimidine

5-tert-Butyl-4H-1,2,4-triazol-3-amine (400 mg, 2.71 mmol) and 2-bromopropanedial (646 mg, 4.07 mmol) are added to acetic acid (5 mL). After being stirred at 60° C. for 3 h, the reaction mixture is concentrated, neutralized with aqueous saturated solution of NaHCO₃, and extracted three times with DCM. The combined organic phases are dried (Na₂SO₄), concentrated, and purified by column chromatography (SiO₂, CyH/EtOAc gradient) to yield the title compound.

C₉H₁₁BrN₄ (M=255.1 g/mol)

ESI-MS: 255/257 [M+H]+

Rt (HPLC): 0.84 min (method D)

### Intermediate III.14

A solution of 6-bromo-2-tert-butyl-[1,2,4]triazolo[1,5-a]pyrimidine (200 mg, 0.63 mmol), bis(pinacolato)diboron (260 mg, 1.02 mmol), and potassium acetate (240 mg, 2.45 mmol) in 1,4-dioxane (4 mL) is purged with Ar for 15 min, followed by addition of bis(triphenylphosphine)palladium chloride (CAS: 13965-03-2) (55 mg, 0.08 mmol). After being stirred at 60° C. for 24 h, the mixture is cooled to ambient temperature, diluted with EtOAc, and filtered through a silica plug. Filtrate is concentrated, redissolved in MeCN/H₂O/TFA, and purified by preparative HPLC (SunFire C18, MeCN/H₂O gradient containing 0.1% TFA) to give intermediate 111.14.

C₉H₁₃BN₄O₂ (M=220.0 g/mol)

ESI-MS: 221 [M+H]+

Rt (HPLC): 0.34 min (method C)

### Synthesis of Intermediate III.15

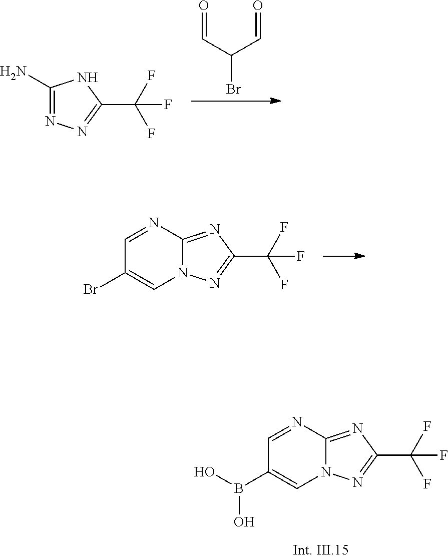 [SMILES: Nc1nnc(C(F)(F)F)[nH]1 ; O=CC(Br)C=O ; FC(F)(F)c1nc2ncc(Br)cn2n1 ; OB(O)c1cnc2nc(C(F)(F)F)nn2c1]

### 6-Bromo-2-(trifluoromethyl)-[1,2,4]triazolo[1,5-a]pyrimidine

5-(Trifluoromethyl)-4H-1,2,4-triazol-3-amine (500 mg, 3.12 mmol) and 2-bromopropanedial (744 mg, 4.69 mmol) are added to acetic acid (5 mL). After being stirred at 60° C. for 3 h, the reaction mixture is concentrated, neutralized with aqueous saturated solution of NaHCO₃, and extracted three times with DCM. The combined organic phases are dried (Na₂SO₄), concentrated, and purified by column chromatography (SiO₂, CyH/EtOAc gradient) to yield the title compound.

C₆H₂BrF₃N₄ (M=267.0 g/mol)

ESI-MS: 267/269 [M+H]+

Rt (HPLC): 0.78 min (method D)

### Intermediate III.15

A solution of 6-bromo-2-(trifluoromethyl)-[1,2,4]triazolo[1,5-a]pyrimidine (155 mg, 0.58 mmol), bis(pinacolato)diboron (200 mg, 0.79 mmol), and potassium acetate (175 mg, 1.78 mmol) in 1,4-dioxane (3 mL) is purged with Ar for 15 min, followed by addition of bis(triphenylphosphine)palladium chloride (CAS: 13965-03-2) (40 mg, 0.06 mmol). After being stirred at 60° C. for 3 h, the mixture is cooled to ambient temperature, diluted with EtOAc, and filtered through a silica plug. Filtrate is concentrated, redissolved in MeCN/H₂O, and purified by preparative HPLC (XBridge C18, MeCN/H₂O gradient containing 0.1% TFA) to give intermediate 111.15.

C₆H₄BF₃N₄O₂ (M=231.9 g/mol)

ESI-MS: 233 [M+H]+

Rt (HPLC): 0.32 min (method C)

### Synthesis of Intermediate III.16 and 111.17

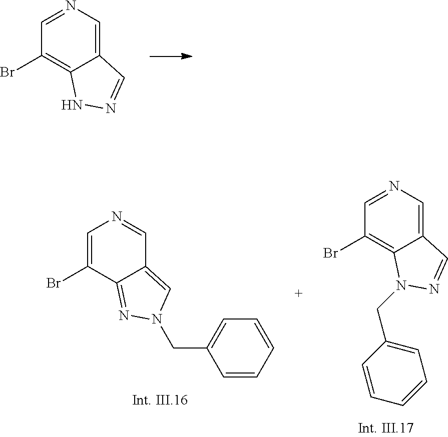 [SMILES: Brc1cncc2cn[nH]c12 ; Brc1cncc2cn(Cc3ccccc3)nc12 ; Brc1cncc2cnn(Cc3ccccc3)c12]

7-Bromo-1H-pyrazolo[4,3-c]pyridine (500 mg, 2.42 mmol) and potassium carbonate (838 mg, 6.06 mmol) are added to THE (10 mL). After 10 minutes, benzyl bromide (353 μL, 2.91 mmol) are added and the resulting reaction mixture is stirred at ambient temperature for 18 h. The reaction mixture is filtered, concentrated and purified by preparative HPLC (X-bridge C₁₈, water/MeCN gradient containing 0.1% NH₃) to yield the desired separated products.

### Int. III.16

C₁₃H₁₀BrN₃ (M=288.1 g/mol)

ESI-MS: 288/290 [M+H]+

Rt (HPLC): 1.06 min (method E)

¹H NMR (400 MHz, DMSO-d₆) δ ppm 9.12 (s, 1H), 8.53 (s, 1H), 8.51 (s, 1H), 7.23-7.35 (m, 3H), 7.08-7.13 (m, 2H), 5.98 (s, 2H).

### Int. III.17

C₁₃H₁₀BrN₃ (M=288.1 g/mol)

ESI-MS: 288/290 [M+H]+

Rt (HPLC): 0.91 min (method E)

¹H NMR (400 MHz, DMSO-d₆) δ ppm 9.15 (s, 1H), 8.97 (s, 1H), 8.36 (s, 1H), 7.31-7.42 (m, 5H), 5.76 (s, 2H).

### Synthesis of Intermediate III.18

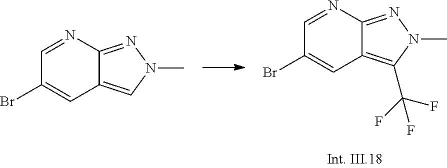

5-Bromo-2-methyl-2h-pyrazolo[3,4-b]pyridine (1.00 g, 4.72 mmol) and zinc(II) trifluoromethanesulfinate (2.35 g, 7.04 mmol) are suspended in a mixture of DCM (50 mL) and water (10 mL). TFA (351 μL, 4.72 mmol) and tert-butyl hydroperoxide (70% in water, 3.26 mL, 23.6 mmol) are added and the resulting reaction mixture is stirred at ambient temperature for 18 h. Zinc(II) trifluoromethanesulfinate (0.50 g, 1.51 mmol) and tert-butyl hydroperoxide (70% in water, 1.00 mL, 7.22 mmol) are added and the reaction mixture is stirred at 45° C. for 2 h. After cooling to ambient temperature, the reaction mixture is diluted with water and the organic phase is separated. The aqueous phase is extracted with DCM. The combined organic extracts are dried over MgSO₄, DMF (10 mL) is added and the mixture is concentrated. The residual DMF solution is purified by preparative HPLC (Sunfire C18, water/ACN gradient containing 0.1% NH₃) to yield the desired product along with other regioisomers from the trifluoromethylation reaction.

C₈H₅BrF₃N₃ (M=280.0 g/mol)

ESI-MS: 280/282 [M+H]+

Rt (HPLC): 1.01 min (method D)

¹H NMR (400 MHz, DMSO-d₆) δ ppm 8.81 (d, J=2.3 Hz, 1H), 8.53 (dq, J=2.2, 1.1 Hz, 1H), 4.35 (q, J=0.9 Hz, 3H)

### Synthesis of Intermediate III.19

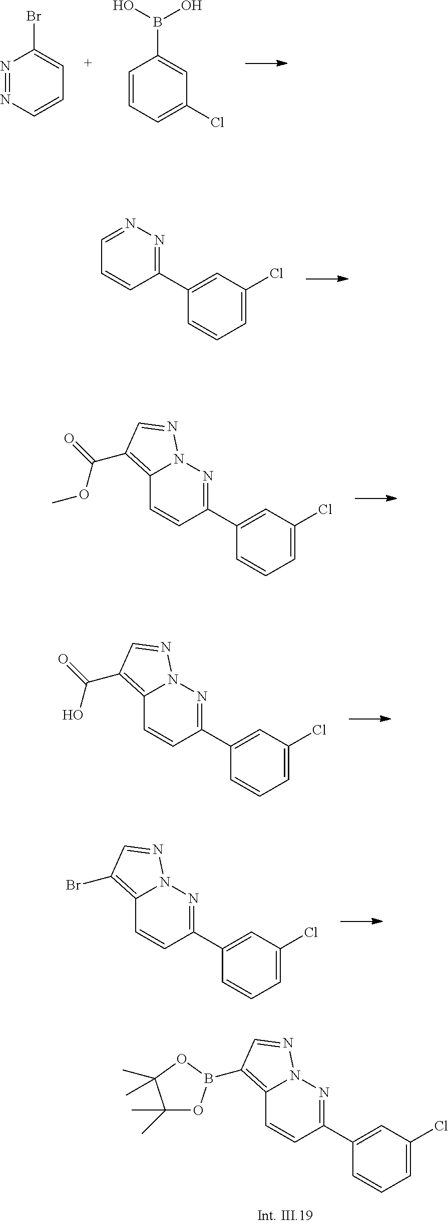

### 3-(3-chlorophenyl)pyridazine

Under a nitrogen atmosphere, (3-chlorophenyl)boronic acid (0.50 g, 3.19 mmol), KOAc (0.72 g, 7.36 mmol) and Pd(dppf)Cl₂*DCM (0.10 g, 0.123 mmol) are added to a mixture of 3-bromopyridazine (0.50 g, 2.45 mmol) in 1,4-dioane (5 mL) and water (1 mL). The resulting reaction mixture was stirred at 100° C. for 12 h. After cooling to ambient temperature, the reaction mixture was diluted with water (50 mL) and extracted with EtOAc (2×100 mL). The combined organic layers were washed with brine (50 mL), dried over Na₂SO₄, filtered and concentrated under reduced pressure. The residue was purified by column chromatography (SiO₂, petroleum ether/EtOAc gradient) to yield 3-(3-chlorophenyl)pyridazine.

C₁₀H₇ClN₂ (M=190.6 g/mol)

ESI-MS: 191 [M+H]+

Rt (HPLC): 0.54 min (method F)

### Methyl 6-(3-chlorophenyl)pyrazolo[1,5-b]pyridazine-3-carboxylate

To a mixture of NH₂OHSO₃ (3.63 g, 32.1 mmol) in H₂O (20 mL) is added sat. aq. NaHCO₃ solution (40 mL) to adjust pH to 6 and heated to 70° C. 3-(3-Chlorophenyl)pyridazine (4.00 g, 21.0 mmol) is added to the mixture. The resulting reaction mixture is stirred at 70° C. for 2 h. After cooling to ambient temperature, the pH value is adjusted to pH 7 and methyl prop-2-ynoate (0.45 g, 5.35 mmol) in DCM (20 mL) is added to the mixture. The resulting mixture is stirred at ambient temperature for 12 h. The reaction mixture is diluted with water and extracted with EtOAc. The combined organic extracts are washed with brine, dried over Na₂SO₄, filtered and concentrated under reduced pressure. The residue is purified by column chromatography (SiO₂, petroleum ether/EtOAc gradient) to yield methyl 6-(3-chlorophenyl)pyrazolo [1,5-b]pyridazine-3-carboxylate.

C₁₄H₁₀ClN₃O₂ (M=287.7 g/mol)

ESI-MS: 288 [M+H]+

Rt (HPLC): 0.74 min (method F)

### 6-(3-chlorophenyl)pyrazolo[1,5-b]pyridazine-3-carboxylic acid

To a mixture of methyl 6-(3-chlorophenyl)pyrazolo[1,5-b]pyridazine-3-carboxylate (5.00 g, 10.4 mmol) in THE (50 mL) is added LiOH (2.00 eq, 0.88 g, 20.9 mmol) and water (30 mL). The resulting reaction mixture is stirred at ambient temperature for 5 h. The reaction mixture is diluted with water and extracted with EtOAc. The aqueous phase is acidified to pH 1 and extracted with EtOAc. The combined organic extracts are washed with brine dried over Na₂SO₄, filtered and concentrated under reduced pressure to give a the desired compound that was used in the next step without further purification.

C₁₃H₈ClN₃O₂ (M=273.7 g/mol)

ESI-MS: 274 [M+H]+

Rt (HPLC): 0.64 min (method F)

### 3-bromo-6-(3-chlorophenyl)pyrazolo[1,5-b]pyridazine

To a mixture of 6-(3-chlorophenyl)pyrazolo[1,5-b]pyridazine-3-carboxylic acid (1.60 g, 5.44 mmol) in DMF (15 mL) is added NBS (1.94 g, 10.9 mmol). The resulting reaction mixture is stirred at ambient temperature for 12 h. The reaction mixture is diluted with water and extracted with EtOAc (2×100 mL). The combined organic extracts are washed with brine, dried over Na₂SO₄, filtered and concentrated under reduced pressure. The residue was purified by column chromatography (SiO₂, petroleum ether/EtOAc gradient) to give the desired product.

C₁₂H₇BrClN₃ (M=308.6 g/mol)

ESI-MS: 308/310 [M+H]+

Rt (HPLC): 0.81 min (method F)

### Intermediate III.19

Under a nitrogen atmosphere, 3-bromo-6-(3-chlorophenyl)pyrazolo[1,5-b]pyridazine (0.40 g, 1.04 mmol) is added to 1,4-dioxane (5 mL) and bis(pinacolato)diboron (1.58 g, 6.22 mmol), Pd(PPh₃)₄ (0.24 g, 0.21 mmol) and KOAc (0.37 g, 3.73 mmol) are added. The resulting reaction mixture is stirred at 100° C. for 12 h. The reaction mixture is diluted with water and extracted with EtOAc (2×). The combined organic extracts are washed with brine, dried over Na₂SO₄, filtered and concentrated under reduced pressure. The residue is purified by column chromatography (SiO₂, petroleum ether/EtOAc gradient).

C₁₈H₁₉BClN₃O₂ (M=355.6 g/mol)

ESI-MS: 356 [M+H]+

Rt (HPLC): 0.90 min (method F)

### Synthesis of Intermediate III.20

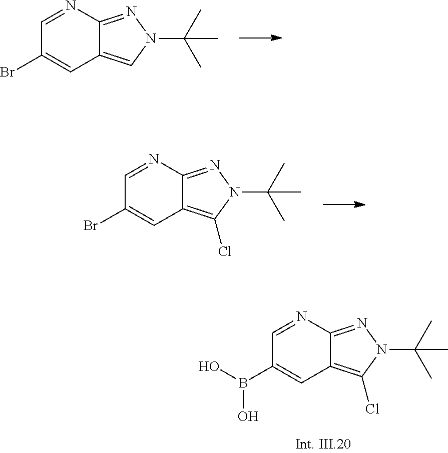 [SMILES: CC(C)(C)n1cc2cc(Br)cnc2n1 ; CC(C)(C)n1nc2ncc(Br)cc2c1Cl ; CC(C)(C)n1nc2ncc(B(O)O)cc2c1Cl]

### 5-Bromo-2-tert-butyl-3-chloro-2H-pyrazolo[3,4-b]pyridine

5-Bromo-2-tert-butyl-2H-pyrazolo[3,4-b]pyridine (1.00 g, 3.93 mmol) is added to acetonitrile (15 mL) and N-chlorosuccinimide (0.58 g, 4.33 mmol) is added at ambient temperature. The resulting reacting mixture is stirred at 85° C. for 12 h. After cooling to ambient temperature, the mixture is concentrated and resuspended in water. It is extracted with EtOAc (3×). The combined organic extracts are dried over Na₂SO₄, filtered and concentrated. The residue is purified by column chromatography (SiO₂, PE/EtOAc gradient) to yield the desired product.

C₁₀H₁₁BrClN₃ (M=288.6 g/mol)

ESI-MS: 288/290 [M+H]+

Rt (HPLC): 0.80 min (Method R)

### Intermediate III.20

A solution of 5-bromo-2-tert-butyl-3-chloro-pyrazolo[3,4-b]pyridine (0.80 g, 2.77 mmol), bis(pinacolato)diboron (0.92 g, 3.61 mmol), and potassium acetate (815 mg, 8.32 mmol) in 1,4-dioxane (16 mL) is purged with N₂ for 10 min, followed by addition of [1,1′-bis-(diphenylphosphino)-ferrocen]-dichloro-palladium(II) (Pd(dppf)Cl₂, CAS: 72287-26-4) (202 mg, 0.23 mmol). After being stirred at 100° C. for 12 h, the mixture is cooled to ambient temperature, concentrated and resuspended in water. It is extracted with EtOAc (3×), dried over Na₂SO₄ and concentrated. The residue is purified via preparative HPLC (Welch Xtimate C₁₈, acetonitrile/water gradient containing 10 mM NH₄HCO₃) to give Int. III.4.

C₁₀H13BClN₃O₂ (M=253.5 g/mol)

ESI-MS: 254 [M+H]+

Rt (HPLC): 0.71 min (Method S)

### Intermediate IV.1

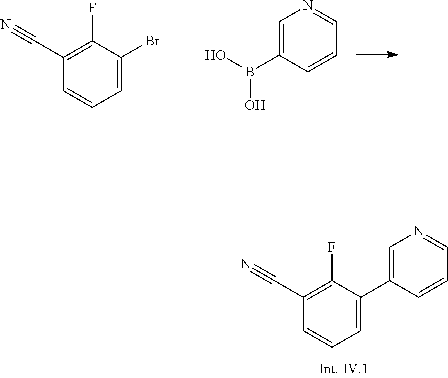 [SMILES: N#Cc1cccc(Br)c1F ; OB(O)c1cccnc1 ; N#Cc1cccc(-c2cccnc2)c1F]

To a mixture of 3-bromo-2-fluorobenzonitrile (500 mg, 2.50 mmol) and pyridine-3-boronic acid (307 mg, 2.50 mmol) in 1,4-dioxane (5 mL) is added sodium carbonate (2 M in H₂O, 2 mL, 4.0 mmol). The resulting mixture is purged with argon for 15 min, followed by addition of [1,1′-Bis-(diphenylphosphino)-ferrocen]-dichloro-palladium(II) (Pd(dppf)Cl₂, CAS: 72287-26-4) (0.091 g, 125 μmol), and the mixture is further purged with argon for 3 min. The reaction mixture is heated to 90° C. and stirred for 3 h. After cooling to ambient temperature, the reaction mixture is diluted with EtOAc and washed with water. The aqueous phase is extracted twice with EtOAc and the combined organic extract is treated with charcoal, filtered through Celite and concentrated. The residue is used in the next step without further purification.

C₁₂H₇FN₂ (M=198.2 g/mol)
EI-MS: 199 [M+H]+

Rt (HPLC): 0.28 min (method C)

### Intermediate V.1

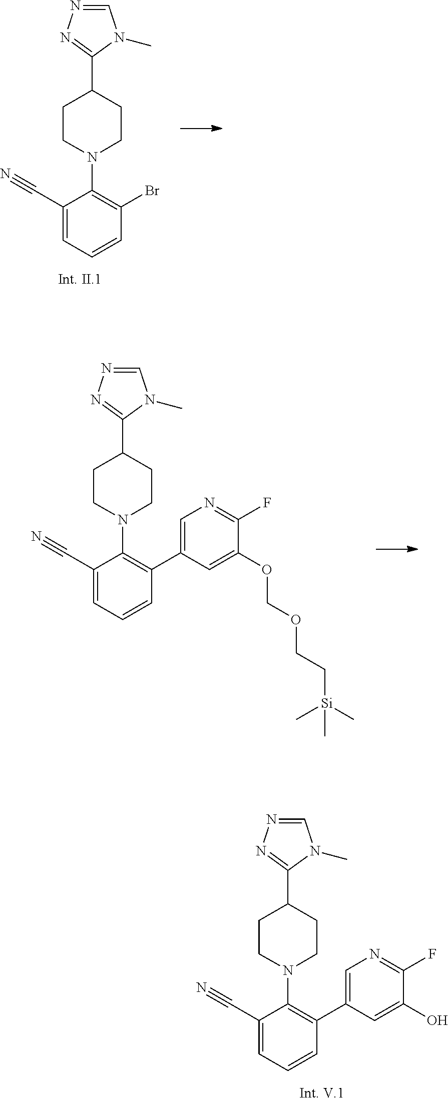 [SMILES: Cn1cnnc1C1CCN(c2c(Br)cccc2C#N)CC1 ; Cn1cnnc1C1CCN(c2c(C#N)cccc2-c2cnc(F)c(OCOCC[Si](C)(C)C)c2)CC1 ; Cn1cnnc1C1CCN(c2c(C#N)cccc2-c2cnc(F)c(O)c2)CC1]

### 3-(6-fluoro-5-{[2-(trimethylsilyl)ethoxy]methoxy}pyridin-3-yl)-2-[4-(4-methyl-4H-1,2,4-triazol-3-yl)piperidin-1-yl]benzonitrile

To a mixture of intermediate 11.1 (2.50 g, 7.22 mmol) and intermediate II.12 (˜65% purity, 5.07 g, 8.9 mmol) in 1,4-dioxane (30 mL) is added cesium carbonate (2 M in H₂O, 11.8 mL, 21.7 mmol). The resulting mixture is purged with argon for 15 min, followed by addition of tetrakis(triphenylphosphine)palladium(0) (0.834 g, 0.722 mmol) is added, and the mixture is further purged with argon for 3 min. The reaction mixture is heated to 80° C. and stirred for 2 h. After cooling to ambient temperature, the reaction mixture is diluted with diethyl ether, washed with half conc. NaCl solution, dried over Na₂SO₄, and concentrated. The crude material is taken into the next step without further purification.

C₂₆H₃₃FN₆O₂Si (M=508.7 g/mol)
EI-MS: 509 [M+H]+

Rt (HPLC): 0.68 min (method C)

### Intermediate V.1

3-(6-fluoro-5-{[2-(trimethylsilyl)ethoxy]methoxy}pyridin-3-yl)-2-[4-(4-methyl-4H-1,2,4-triazol-3-yl)piperidin-1-yl]benzonitrile (˜65% purity, 6.77 g, 8.65 mmol) is added to 1,4-dioxane (60 mL) and a solution of HCl in 1,4-dioxane (4 M, 16.6 mL, 66.5 mmol) is added. The resulting reaction mixture is stirred for 66 h at ambient temperature. The mixture is concentrated and the residue purified by preparative HPLC (XBridge C18, ACN/water gradient 0.1% NH₃) to yield the desired product.

C₂₀H₁₉FN₆O (M=378.4 g/mol)
EI-MS: 379 [M+H]+

Rt (HPLC): 0.42 min (method C)

### Intermediate V.2

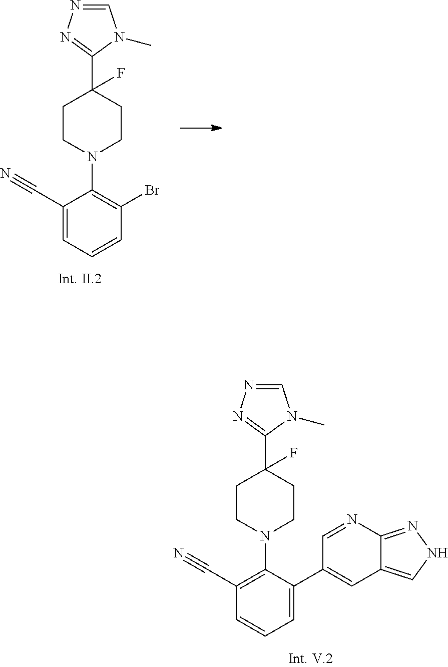 [SMILES: Cn1cnnc1C1(F)CCN(c2c(Br)cccc2C#N)CC1 ; Cn1cnnc1C1(F)CCN(c2c(C#N)cccc2-c2cnc3n[nH]cc3c2)CC1]

To a mixture of intermediate II.2 (110 mg, 302 μmol) and (1H-pyrazolo[3,4-b]pyridine-5-yl)boronic acid (62.2 mg, 362 μmol) in 1,4-dioxane (5 mL) is added potassium carbonate (2 M in H₂O, 302 μL, 604 μmol). The resulting mixture is purged with argon for 15 min, followed by addition of [1,1′-Bis-(diphenylphosphino)-ferrocen]-dichloro-palladium(II) (Pd(dppf)Cl₂, CAS: 72287-26-4) (22.1 mg, 30.2 μmol), and the mixture is further purged with argon for 3 min. The reaction mixture is heated to 100° C. and stirred for 1 h. After cooling to ambient temperature, the reaction mixture is diluted with EtOAc, filtered through Celite and concentrated. The residue is purified via preparative HPLC (XBridge C18, ACN/water gradient containing 0.1% TFA) to yield the desired compound.

C₂₁H₁₉FN₈ (M=402.4 g/mol)

ESI-MS: 403 [M+H]+

Rt (HPLC): 0.75 min (method E)

### Preparation of Final Compounds

### Example 1

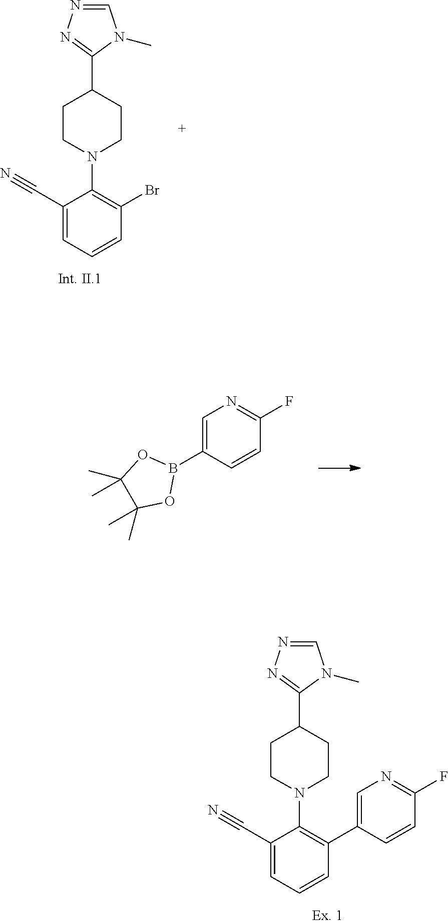 [SMILES: Cn1cnnc1C1CCN(c2c(Br)cccc2C#N)CC1 ; CC1(C)OB(c2ccc(F)nc2)OC1(C)C ; Cn1cnnc1C1CCN(c2c(C#N)cccc2-c2ccc(F)nc2)CC1]

To a mixture of intermediate 11.1 (750 mg, 2.2 mmol) and 2-fluoropyridine-5-boronic acid pinacol ester (CAS: 329214-79-1) (580 mg, 2.6 mmol) in 1,4-dioxane (15 mL) is added potassium carbonate (2 M in H₂O, 3.2 mL, 6.5 mmol). The resulting mixture is purged with argon for 15 min, [1,1′-bis-(diphenylphosphino)-ferrocen]-dichloro-palladium(II) (Pd(dppf)Cl₂, CAS: 72287-26-4) (158 mg, 0.22 mmol) is added, and the mixture is further purged with argon for 3 min. The reaction mixture is heated to 100° C. and stirred for 2 h. After cooling to ambient temperature, the reaction mixture is diluted with EtOAc, washed with H₂O and saturated aqueous solution of NaHCO₃, dried over Na₂SO₄, and concentrated. The crude product is purified via preparative HPLC (XBridge C18, ACN/water gradient containing 0.1% NH₃) to yield the desired compound.

C₂₀H₁₉FN₆ (M=362.4 g/mol)

ESI-MS: 363 [M+H]+

Rt (HPLC): 0.42 min (method C)

¹H NMR (400 MHz, DMSO-d6) δ=9.35 (s, 1H), 8.27 (d, J=2.2 Hz, 1H), 8.08 (dt, J=2.3, 8.2 Hz, 1H), 7.80 (dd, J=1.6, 7.7 Hz, 1H), 7.57 (dd, J=1.5, 7.6 Hz, 1H), 7.37-7.27 (m, 2H), 3.79 (s, 3H), 3.28-3.09 (m, 3H), 2.99 (br s, 2H), 1.86 (br d, J=10.8 Hz, 2H), 1.73-1.58 (m, 2H)

The following examples were prepared with minor modifications of the procedure of example 1:

| | Starting | | |
|---|---|---|---|
| Ex. | material | Structure | Deviation from general procedure |
| 2 | Int. II.1 + 4-(4,4,5,5-tet- ramethyl-1,3,2- dioxaborolan- 2-yl)isothiazole | 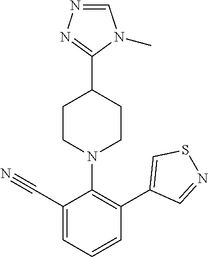 [SMILES: Cn1cnnc1C1CCN(c2c(C#N)cccc2-c2cnsc2)CC1] | 1.3 eq boronic acid derivative; 3.5 eq K₃PO₄ solution 0.1 XPhos Pd G3 (CAS: 1445085- 55-1); 18 h at 100° C. |
| 3 | Int. II.1 + 2-(2-methoxy- ethoxy)-5- (4,4,5,5-tetra- methyl-1,3,2- dioxaborolan- 2-yl)pyrimi- dine | 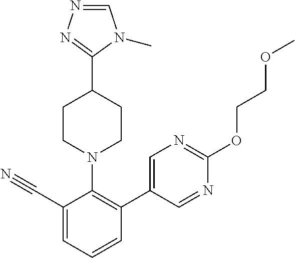 [SMILES: COCCOc1ncc(-c2cccc(C#N)c2N2CCC(c3nncn3C)CC2)cn1] | 1.3 eq boronic acid derivative; 3.5 eq K₃PO₄ solution 0.1 XPhos Pd G3 (CAS: 1445085- 55-1); 18 h at 100° C. |
| 4 | Int. II.1 + 2-fluoro-4- methylpyri- dine-5-boronic acid | 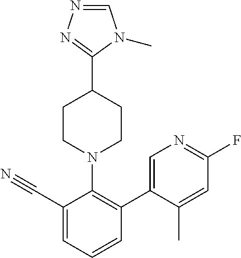 [SMILES: Cc1cc(F)ncc1-c1cccc(C#N)c1N1CCC(c2nncn2C)CC1] | 1.3 eq boronic acid derivative; 3.5 eq K₃PO₄ solution 0.1 XPhos Pd G3 (CAS: 1445085- 55-1); 18 h at 100° C. |
| 5 | Int. II.1 + 2-fluoro-3- methylpyri- dine-5-boronic acid | 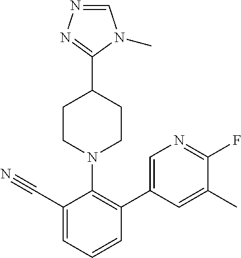 [SMILES: Cc1cc(-c2cccc(C#N)c2N2CCC(c3nncn3C)CC2)cnc1F] | 1.3 eq boronic acid derivative; 3.5 eq K₃PO₄ solution 0.1 XPhos Pd G3 (CAS: 1445085- 55-1); 18 h at 100° C. |
| 6 | Int. II.1 + 5-(1H-pyrazol- 1-yl)pyridine- 3-boronic acid | 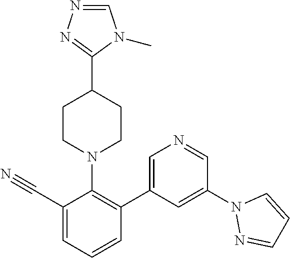 [SMILES: Cn1cnnc1C1CCN(c2c(C#N)cccc2-c2cncc(-n3cccn3)c2)CC1] | 1.3 eq boronic acid derivative; 3.5 eq K₃PO₄ solution 0.1 XPhos Pd G3 (CAS: 1445085- 55-1); 18 h at 100° C. |
| 7 | Int. II.1 + 6-(4,4,5,5-tet- ramethyl-1,3,2- dioxaborolan- 2-yl)- [1,2,5]oxadia- zolo[3,4-b]pyri- dine | 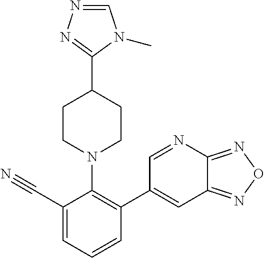 [SMILES: Cn1cnnc1C1CCN(c2c(C#N)cccc2-c2cnc3nonc3c2)CC1] | 1.3 eq boronic acid derivative; 3.5 eq K₃PO₄ solution 0.1 XPhos Pd G3 (CAS: 1445085- 55-1); 18 h at 100° C. |
| 8 | Int. II.1 + 4-methyl-3- (4,4,5,5-tetra- methyl-1,3,2- dioxaborolan- 2-yl)pyridine | 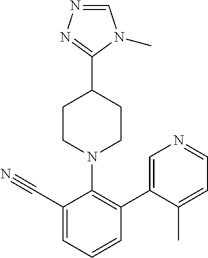 [SMILES: Cc1ccncc1-c1cccc(C#N)c1N1CCC(c2nncn2C)CC1] | 1.3 eq boronic acid derivative; 3.5 eq K₃PO₄ solution 0.1 XPhos Pd G3 (CAS: 1445085- 55-1); 18 h at 100° C. |
| 9 | Int. II.1 + (5-(hy- droxymeth- yl)pyridin-3- yl)boronic acid | 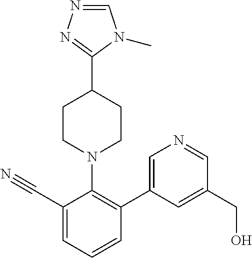 [SMILES: Cn1cnnc1C1CCN(c2c(C#N)cccc2-c2cncc(CO)c2)CC1] | 1.3 eq boronic acid derivative; 3.5 eq K₃PO₄ solution 0.1 XPhos Pd G3 (CAS: 1445085- 55-1); 18 h at 100° C. |
| 10 | Int. II.1 + 5-(prop-1- ynyl)pyridin-3- ylboronic acid | 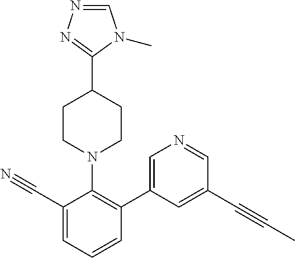 [SMILES: CC#Cc1cncc(-c2cccc(C#N)c2N2CCC(c3nncn3C)CC2)c1] | 1.3 eq boronic acid derivative; 3.5 eq K₃PO₄ solution 0.1 XPhos Pd G3 (CAS: 1445085- 55-1); 18 h at 100° C. |
| 11 | Int. II.1 + 3-methyl-5- (4,4,5,5-tetra- methyl-1,3,2- dioxaborolan- 2-yl)pyridine | 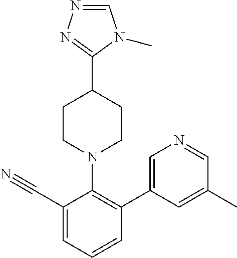 [SMILES: Cc1cncc(-c2cccc(C#N)c2N2CCC(c3nncn3C)CC2)c1] | 1.3 eq boronic acid derivative; 3.5 eq K₃PO₄ solution 0.1 XPhos Pd G3 (CAS: 1445085- 55-1); 18 h at 100° C. |
| 12 | Int. II.1 + 6-(4,4,5,5-tet- ramethyl-1,3,2- dioxaborolan- 2-yl)-3,4-dihy- dro-2H-py- rano[2,3-b]pyri- dine | 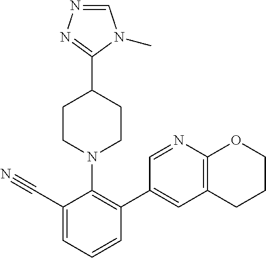 [SMILES: Cn1cnnc1C1CCN(c2c(C#N)cccc2-c2cnc3c(c2)CCCO3)CC1] | 1.3 eq boronic acid derivative; 3.5 eq K₃PO₄ solution 0.1 XPhos Pd G3 (CAS: 1445085- 55-1); 18 h at 100° C. |
| 13 | Int. II.1 + pyrimidine-5- boronic acid | 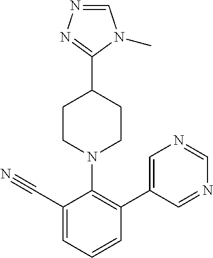 [SMILES: Cn1cnnc1C1CCN(c2c(C#N)cccc2-c2cncnc2)CC1] | 3 equiv of 2 M Na₂CO₃ aq. solu- tion; 5 mol % Pd(dppf)Cl₂; 80° C. for 16 h; purified by preparative HPLC (XBridg C18, ACN/water gradient containing 0.1% TFA) |
| 14 | Int. II.1 + 5-(4,4,5,5-tet- ramethyl-1,3,2- dioxaborolan- 2-yl)nicotina- mide | 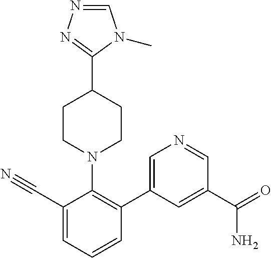 [SMILES: Cn1cnnc1C1CCN(c2c(C#N)cccc2-c2cncc(C(N)=O)c2)CC1] | 1.3 eq boronic acid derivative; 3.5 eq K₃PO₄ solution 0.1 XPhos Pd G3 (CAS: 1445085- 55-1); 18 h at 100° C. |
| 15 | Int. II.1 + 3-cyclopropyl- 5-(4,4,5,5-tet- ramethyl-1,3,2- dioxaborolan- 2-yl)pyridine | 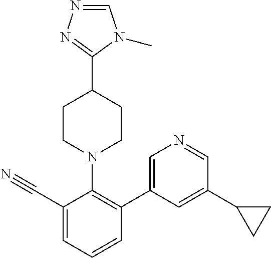 [SMILES: Cn1cnnc1C1CCN(c2c(C#N)cccc2-c2cncc(C3CC3)c2)CC1] | 1.3 eq boronic acid derivative; 3.5 eq K₃PO₄ solution 0.1 XPhos Pd G3 (CAS: 1445085- 55-1); 18 h at 100° C. |
| 16 | Int. II.1 + 5-methoxypyri- dine-3-boronic acid | 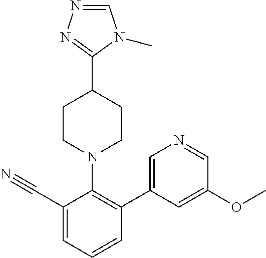 [SMILES: COc1cncc(-c2cccc(C#N)c2N2CCC(c3nncn3C)CC2)c1] | 1.3 eq boronic acid derivative; 3.5 eq K₃PO₄ solution 0.1 XPhos Pd G3 (CAS: 1445085- 55-1); 18 h at 100° C. |
| 17 | Int. II.1 + 5-fluoro- pyridine-3-bo- ronic acid | 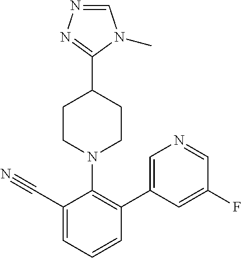 [SMILES: Cn1cnnc1C1CCN(c2c(C#N)cccc2-c2cncc(F)c2)CC1] | 1.3 eq boronic acid derivative; 3.5 eq K₃PO₄ solution 0.1 XPhos Pd G3 (CAS: 1445085- 55-1); 18 h at 100° C. |
| 18 | Int. II.1 + 2-chloro-5- (4,4,5,5-tetra- methyl-1,3,2- dioxaborolan- 2-yl)pyridine | 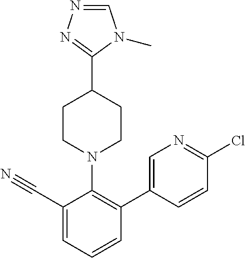 [SMILES: Cn1cnnc1C1CCN(c2c(C#N)cccc2-c2ccc(Cl)nc2)CC1] | 1.3 eq boronic acid derivative; 3.5 eq K₃PO₄ solution 0.1 XPhos Pd G3 (CAS: 1445085- 55-1); 18 h at 100° C. |
| 19 | Int. II.1 + 2-fluoro-3-pro- pan-2-yloxy-5- (4,4,5,5-tetra- methyl-1,3,2- dioxaborolan- 2-yl)pyridine | 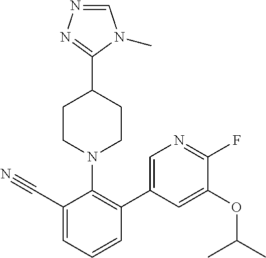 [SMILES: CC(C)Oc1cc(-c2cccc(C#N)c2N2CCC(c3nncn3C)CC2)cnc1F] | 3 equiv of 2 M Na₂CO₃ aq. solu- tion; 5 mol % Pd(dppf)Cl₂; 80° C. for 16 h |
| 20 | Int. II.1 + (5,6-difluoro- pyridin-3- yl)boronic acid | 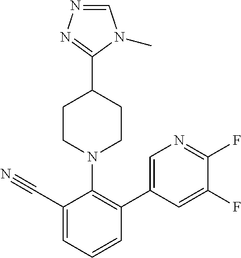 [SMILES: Cn1cnnc1C1CCN(c2c(C#N)cccc2-c2cnc(F)c(F)c2)CC1] | 3 equiv of 2 M Na₂CO₃ aq. solu- tion; 5 mol % Pd(dppf)Cl₂; 80° C. for 16 h |
| 21 | Int. II.1 + (5-phenylpyri- din-3-yl)bo- ronic acid | 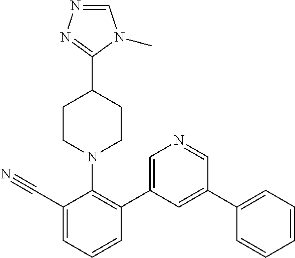 [SMILES: Cn1cnnc1C1CCN(c2c(C#N)cccc2-c2cncc(-c3ccccc3)c2)CC1] | 3 equiv of 2 M Na₂CO₃ aq. solu- tion; 5 mol % Pd(dppf)Cl₂; 80° C. for 16 h |
| 22 | Int. II.1 + (2-methyl-2h- pyrazolo[3,4- b]pyridin-5- yl)boronic acid | 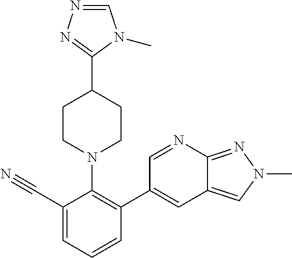 [SMILES: Cn1cc2cc(-c3cccc(C#N)c3N3CCC(c4nncn4C)CC3)cnc2n1] | 2 equiv of 2 M K₂CO₃ aq. solu- tion; 100° C. for 1 h; purified by preparative HPLC (XBridge C18, ACN/water gradient containing 0.1% TFA) |
| 23 | Int. II.1 + 3-(difluoro- methoxy)-5- (4,4,5,5-tetra- methyl-1,3,2- dioxaborolan- 2-yl)pyridine | 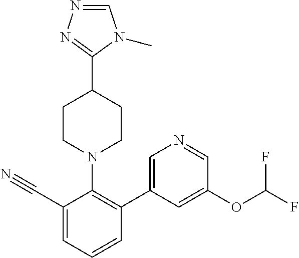 [SMILES: Cn1cnnc1C1CCN(c2c(C#N)cccc2-c2cncc(OC(F)F)c2)CC1] | 2 equiv of 2 M K₂CO₃ aq. solu- tion; 100° C. for 1 h; purified by preparative HPLC (XBridge C18, ACN/water gradient containing 0.1% TFA) |
| 24 | Int. II.1 + 7-(4,4,5,5-tet- ramethyl- [1,3,2]dioxabo- rolan-2-yl)-2,3- dihydro-1h- pyrido[2,3- b][1,4]oxazine | 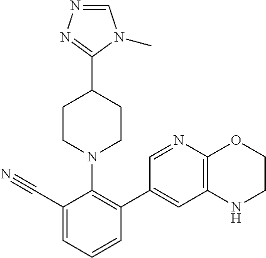 [SMILES: Cn1cnnc1C1CCN(c2c(C#N)cccc2-c2cnc3c(c2)NCCO3)CC1] | 2 equiv of 2 M K₂CO₃ aq. solu- tion; 100° C. for 1 h; purified by preparative HPLC (XBridge C18, ACN/water gradient containing 0.1% TFA) & HPLC (XBridge C18, ACN/water gradient contain- ing 0.1% NH₃) |
| 25 | Int. II.1 + {2H-[1,3]diox- olo[4,5-b]pyri- dine-6-yl}bo- ronic acid | 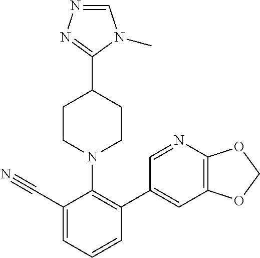 [SMILES: Cn1cnnc1C1CCN(c2c(C#N)cccc2-c2cnc3c(c2)OCO3)CC1] | 2 equiv of 2 M K₂CO₃ aq. solu- tion; 100° C. for 1 h; purified by preparative HPLC (XBridge C18, ACN/water gradient containing 0.1% TFA) & HPLC (SunFire C18, ACN/water gradient contain- ing 0.1% NH₃) |
| 26 | Int. II.1 + (1H-pyra- zolo[3,4-b]pyr- din-5-yl)bo- ronic acid | 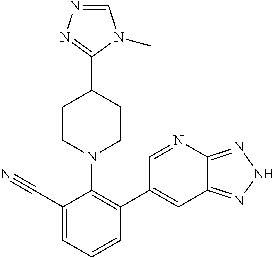 [SMILES: Cn1cnnc1C1CCN(c2c(C#N)cccc2-c2cnc3n[nH]nc3c2)CC1] | 2 equiv of 2 M K₂CO₃ aq. solu- tion; 100° C. for 1 h; purified by preparative HPLC (XBridge C18, ACN/water gradient containing 0.1% TFA) & HPLC (SunFire C18, ACN/water gradient contain- ing 0.1% NH₃) |
| 27 | Int. II.1 + 5-(methoxycar- bonyl)pyridine- 3-boronic acid | 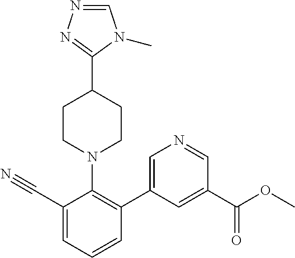 [SMILES: COC(=O)c1cncc(-c2cccc(C#N)c2N2CCC(c3nncn3C)CC2)c1] | 2 equiv of 2 M Na₂CO₃ aq. solu- tion; 100° C. for 1 h; teated with SiliaMetS Thiourea scavenger; purified by preparative HPLC (SunFire C18, ACN/water gradi- ent containing 0.1% TFA) & col- umn chromatography (SiO2, EtOAc/MeOH gradient 1/0 to 3/1) |
| 29 | Int. II.1 + 2-(4-(4,4,5,5- tetramethyl- 1,3,2-dioxabo- rolan-2-yl)pyr- din-3-yl)oxa- zole | 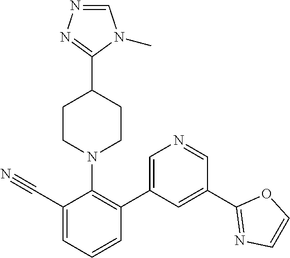 [SMILES: Cn1cnnc1C1CCN(c2c(C#N)cccc2-c2cncc(-c3ncco3)c2)CC1] | 1.6 eq. of pinacol boronic ester, 3 eq. of 2 M Cs₂CO₃ solution; 80° C. for 1.5 h; |
| 30 | Int. II.1 + Int. III.1 | 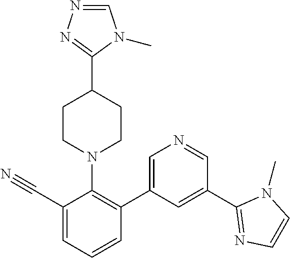 [SMILES: Cn1ccnc1-c1cncc(-c2cccc(C#N)c2N2CCC(c3nncn3C)CC2)c1] | 1.5 eq. of pinacol boronic aester, 3 eq. of 2 M Cs₂CO₃ solution; 80° C. for 1 h; purified by preparative HPLC (XBridge C18, ACN/water gradient containing 0.1% NH3) and preparative SFC (Torus_2- PIC, MeOH/CO₂) |
| 31 | Int. II.1 + Int. III.2 | 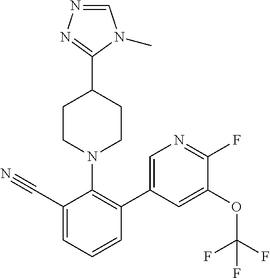 [SMILES: Cn1cnnc1C1CCN(c2c(C#N)cccc2-c2cnc(F)c(OC(F)(F)F)c2)CC1] | 1.4 eq. of pinacol boronic ester, 3 eq. of 2 M Cs₂CO₃ solution; 80° C. for 1 h; |
| 32 | Int. II.1 + 4-(4,4,5,5-tet- ramethyl-1,3,2- dioxaborolan- 2-yl)pyridazine | 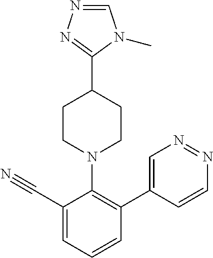 [SMILES: Cn1cnnc1C1CCN(c2c(C#N)cccc2-c2ccnnc2)CC1] | 3 eq. of 2 M Na₂CO₃ solution; 110° C. for 5 h and 120° C. for 5 h; purifed by preparative HPLC (Sun- Fire C18, ACN/water gradient containing 0.1% TFA) |
| 33 | Int. II.1 + 3H-imida- zo[4,5-b]pyri- dine-6-boronic acid | 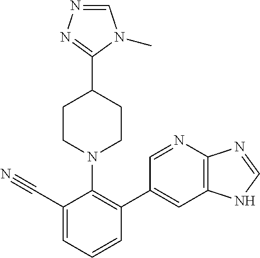 [SMILES: Cn1cnnc1C1CCN(c2c(C#N)cccc2-c2cnc3nc[nH]c3c2)CC1] | 3 eq. of 2 M Na₂CO₃ solution; 110° C. for 5 h and 120° C. for 5 h; purified by preparative HPLC (Sun- Fire C18, ACN/water gradient containing 0.1% TFA) |
| 39 | Int. II.1 + 3-(tetrahydro- 2h-pyran-2-yl)- (4,4,5,5-tetra- methyl-1,3,2- dioxaborolan- 2-yl)pyridine | 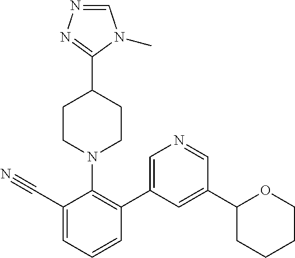 [SMILES: Cn1cnnc1C1CCN(c2c(C#N)cccc2-c2cncc(C3CCCCO3)c2)CC1] | 1.5 eq. of boronic acid derivative; 3 eq. of 2 M Na₂CO₃ solution; 110° C. for 5 h and 120° C. for 7 h; purified by preparative HPLC (Sun- Fire C18, ACN/water gradient containing 0.1% TFA) |
| 40 | Int. II.1 + Int. III.3a | 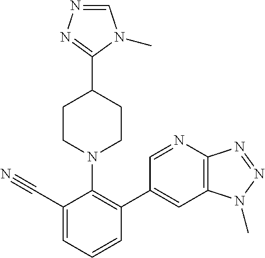 [SMILES: Cn1cnnc1C1CCN(c2c(C#N)cccc2-c2cnc3nnn(C)c3c2)CC1] | 1.0 eq. of Int. III.3a; 2.0 eq. of K₂CO₃ solution; 1 h at 100° C. |
| 45 | Int. II.1 + Int. III.3b | 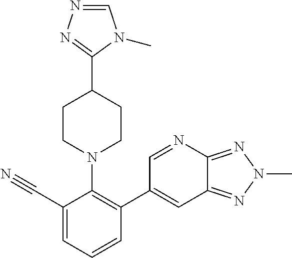 [SMILES: Cn1nc2cc(-c3cccc(C#N)c3N3CCC(c4nncn4C)CC3)cnc2n1] | 1 h at 100° C.; purified by prepara- tive HPLC (XBridge C18, ACN/water gradient containing 0.1% TFA) and chiral preparative SFC (CHIRAL ART ® Amylose- SA, 20 × 25 mm, 5 μm, 75% sCO2, 25% IPA,) |
| 46 | Int. II.1 + Int. III.4 | 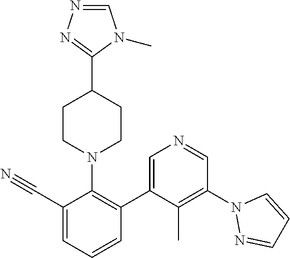 [SMILES: Cc1c(-c2cccc(C#N)c2N2CCC(c3nncn3C)CC2)cncc1-n1cccn1] | 2.0 eq. of K₂CO₃ solution; 10 h at 100° C.; purified by preparative HPLC (XBridge C18, MeCN/wa- ter containing 0.1% NH₃) and pre- parative HPLC (XBridge C18, MeCN/water containing 0.1% TFA) |
| 49 | Int. II.1 + 2-fluoro-3- methoxypyri- dine-5-boronic acid | 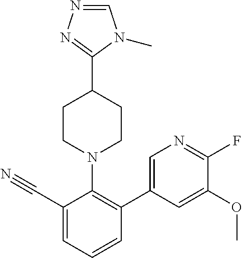 [SMILES: COc1cc(-c2cccc(C#N)c2N2CCC(c3nncn3C)CC2)cnc1F] | 3.0 eq. of Na2CO3 solution is used; 18 h at 80° C.; purified by column chromatography (SiO2, EtOAc/MeOH gradient) |
| 50 | Int. II.1 + Int. III.5 | 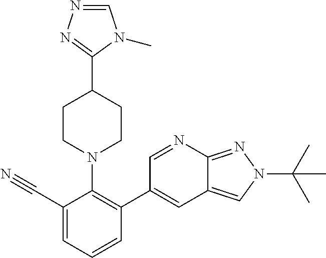 [SMILES: Cn1cnnc1C1CCN(c2c(C#N)cccc2-c2cnc3nn(C(C)(C)C)cc3c2)CC1] | 3.0 eq. of Cs₂CO₃ solution is used; 0.1 eq. Pd(PPh₃)₄; 3 h at 80° C.; purified by column chromatogra- phy (SiO₂, EtOAc/MeOH gradi- ent). |
| 51 | Int. II.1 + Int. III.6 | 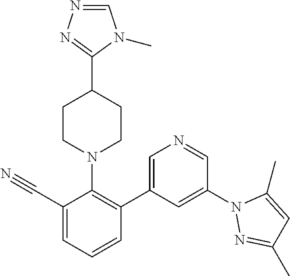 [SMILES: Cc1cc(C)n(-c2cncc(-c3cccc(C#N)c3N3CCC(c4nncn4C)CC3)c2)n1] | 3.0 eq. of Cs₂CO₃ solution is used; 0.1 eq. Pd(PPh₃)₄; 3 h at 80° C. |
| 52 | Int. II.2 + pyridazine-4- boronic acid pinacol ester | 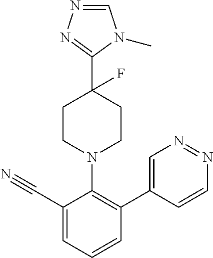 [SMILES: Cn1cnnc1C1(F)CCN(c2c(C#N)cccc2-c2ccnnc2)CC1] | 3.0 eq. of Na₂CO₃ solution is used; 0.1 eq. Pd(PPh₃)₄; 5 h at 110° C. |
| 53 | Int. II.2 + 2-fluoro-3- methoxypyri- dine-5-boronic acid | 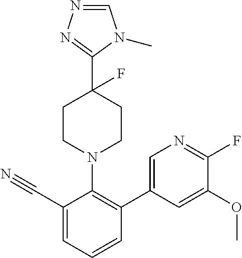 [SMILES: COc1cc(-c2cccc(C#N)c2N2CCC(F)(c3nncn3C)CC2)cnc1F] | 2.0 eq. 2 fluoro-3-methoxypyri- dine-5-boronic acid; 2.5 eq. of Na₂CO₃ solution; 0.1 eq. Pd(PPh₃)₄ is used; 2 h at 110° C. |
| 54 | Int. II.2 + (2-hydroxypy- rimidin-5- yl)boronic acid | 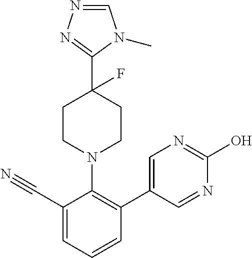 [SMILES: Cn1cnnc1C1(F)CCN(c2c(C#N)cccc2-c2cnc(O)nc2)CC1] | 2.0 eq. (2-hydroxypyrimidin-5- yl)boronic acid; 3.0 eq. of Na₂CO₃ solution;_{;} 0.1 eq. Pd(PPh₃)₄ is used; 18 h at 100° C. |
| 56 | Int. II.2 + 2-fluoro- pyridine-5-bo- ronic acid pina- col ester | 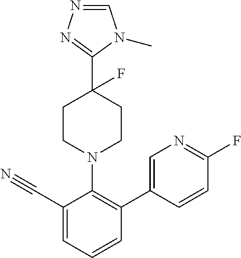 [SMILES: Cn1cnnc1C1(F)CCN(c2c(C#N)cccc2-c2ccc(F)nc2)CC1] | 4.0 eq. of K₂CO₃ solution; 1 h at 100° C.; purified by preparative HPLC (XBridge C18, ACN/water gradi- ent containing 0.1% TFA) |
| 61 | Int. II.2 + Int. III.7 | 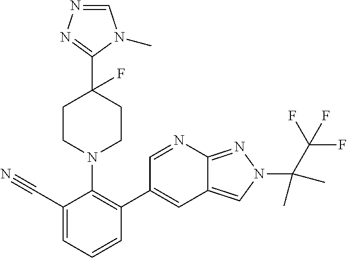 [SMILES: Cn1cnnc1C1(F)CCN(c2c(C#N)cccc2-c2cnc3nn(C(C)(C)C(F)(F)F)cc3c2)CC1] | 2.0 eq. of K₂CO₃ solution is used; 1 h at 100° C. |
| 62 | Int. II.2 + Int. III.5 | 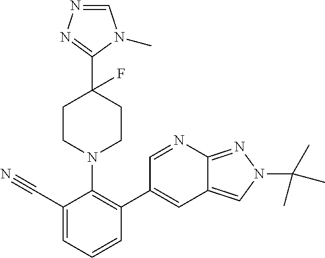 [SMILES: Cn1cnnc1C1(F)CCN(c2c(C#N)cccc2-c2cnc3nn(C(C)(C)C)cc3c2)CC1] | 2.0 eq. of K₂CO₃ solution is used; 3 h at 100° C. |
| 63 | Int. II.2 + Int. III.8a | 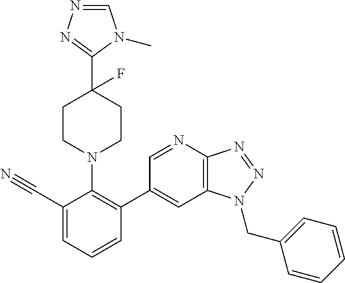 [SMILES: Cn1cnnc1C1(F)CCN(c2c(C#N)cccc2-c2cnc3nnn(Cc4ccccc4)c3c2)CC1] | 1.5 eq. of Int. III.8a is used; 2.0 eq. of K₂CO₃ solution is used; 1 h at 100° C. |
| 64 | Int. II.2 + Int. III.8b | 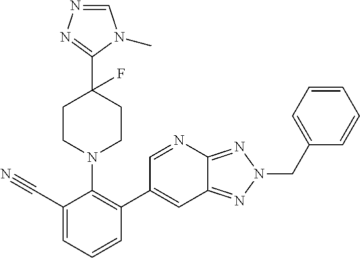 [SMILES: Cn1cnnc1C1(F)CCN(c2c(C#N)cccc2-c2cnc3nn(Cc4ccccc4)nc3c2)CC1] | 1.5 eq. of Int. III.8a is used; 2.0 eq. of K₂CO₃ solution is used; 1 h at 100° C. |
| 66 | Int. II.3 + 2-fluoro- pyridine-5-bo- ronic acid pina- col ester | 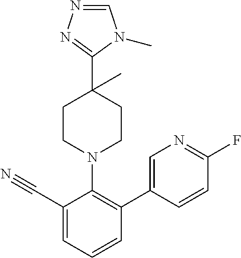 [SMILES: Cn1cnnc1C1(C)CCN(c2c(C#N)cccc2-c2ccc(F)nc2)CC1] | 4 eq. of K₂CO₃ solution; 1 h at 100° C. |
| 67 | Int. II.1 + pyridine-5-bo- ronic acid pina- col ester | 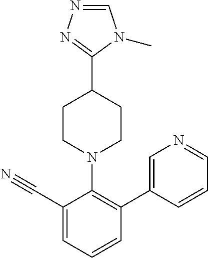 [SMILES: Cn1cnnc1C1CCN(c2c(C#N)cccc2-c2cccnc2)CC1] | 3 eq. of Cs₂CO₃ solution; 0.1 eq. Pd(PPh₃)₄ is used; 1.5 h at 90° C. |
| 70 | Int. II.2 + 3-(4,4,5,5-tet- ramethyl-1,3,2- dioxaborolan- 2- yl)pyra- zolo[1,5-b]pyri- dazine | 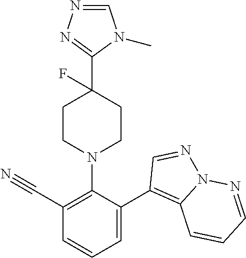 [SMILES: Cn1cnnc1C1(F)CCN(c2c(C#N)cccc2-c2cnn3ncccc23)CC1] | 2 eq. of K₃PO₄ solution is used, 0.1 eq XPhos Pd G3 (CAS: 1445085-55-1) is used; 16 h at 90° C.; purified by prepara- tive HPLC (Sunfire C18, ACN/water gradient containing 0.1% TFA) |
| 71 | Int. II.2 + Int. III.10 | 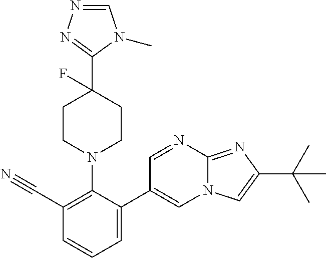 [SMILES: Cn1cnnc1C1(F)CCN(c2c(C#N)cccc2-c2cnc3nc(C(C)(C)C)cn3c2)CC1] | 1.5 eq. of Int. III.10; 3 eq. of K₂CO₃ solution ; 1 h at 100° C.; pu- rified by preparative HPLC (Sun- fire C18, ACN/water gradient containing 0.1% TFA) and prepa- rative HPLC (XBridge C18, ACN/water gradient containing 0.1% NH₃) |
| 72 | Int. II.2 + Int. III.14 | 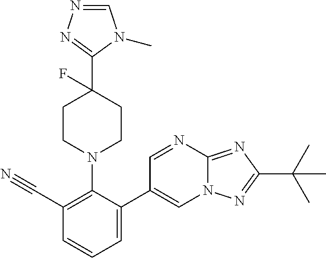 [SMILES: Cn1cnnc1C1(F)CCN(c2c(C#N)cccc2-c2cnc3nc(C(C)(C)C)nn3c2)CC1] | 1.16 eq. of Int. III.14; 2.0 eq. of K₂CO₃ solution; 4 h at 95° C.; pu- rified by preparative HPLC (Sun- fire C18, ACN/water gradient containing 0.1% TFA) |
| 73 | Int. II.2 + Int. III.15 | 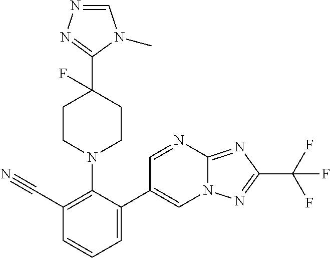 [SMILES: Cn1cnnc1C1(F)CCN(c2c(C#N)cccc2-c2cnc3nc(C(F)(F)F)nn3c2)CC1] | 1.1 eq. of Int. III.15; 2.0 eq. of K₂CO₃ solution; 10 h at 95° C.; pu- rified by preparative HPLC (Sun- fire C18, ACN/water gradient containing 0.1% TFA) |
| 74 | Int. II.2 + Int. III.11 | 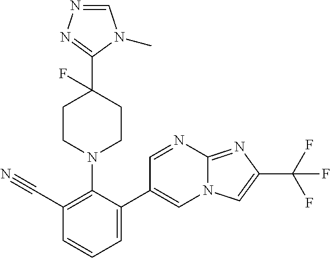 [SMILES: Cn1cnnc1C1(F)CCN(c2c(C#N)cccc2-c2cnc3nc(C(F)(F)F)cn3c2)CC1] | 1.4 eq of Int. III.11; 1 h at 100° C. |
| 75 | Int. II.2 + Int. III.13 | 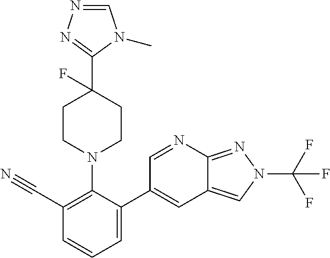 [SMILES: Cn1cnnc1C1(F)CCN(c2c(C#N)cccc2-c2cnc3nn(C(F)(F)F)cc3c2)CC1] | 1.04 eq of Int. III.13; 2 eq. of K₂CO₃ solution; 5 h at 100° C.; ad- ditionally purified by preparative SFC (Torus Diol, MeOH/CO₂) |
| 79 | Int. II.2 + Int. III.19 | 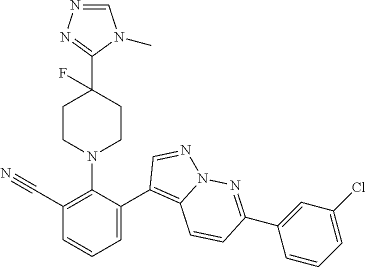 [SMILES: Cn1cnnc1C1(F)CCN(c2c(C#N)cccc2-c2cnn3nc(-c4cccc(Cl)c4)ccc23)CC1] | 4.0 eq. of K₂CO₃ solution; 1 h at 70° C. |
| 81 | Int. II.1 + Int. III.19 | 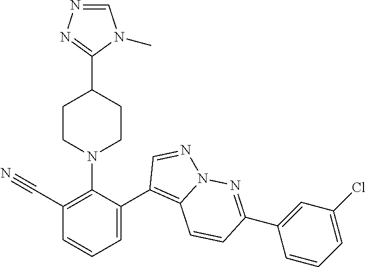 [SMILES: Cn1cnnc1C1CCN(c2c(C#N)cccc2-c2cnn3nc(-c4cccc(Cl)c4)ccc23)CC1] | 4.0 eq. of K₂CO₃ solution; 1 h at 70° C. |
| 82 | Int. II.2 + 2-fluoro-3- methylpyri- dine-5-boronic acid | 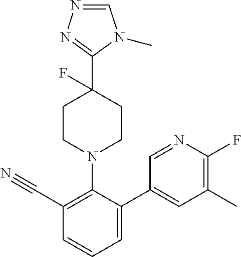 [SMILES: Cc1cc(-c2cccc(C#N)c2N2CCC(F)(c3nncn3C)CC2)cnc1F] | 1.1 eq of 2-fluoro-3-methylpyri- dine-5-boronic acid; 4.0 eq. of K₂CO₃ solution; 2 h at 70° C. |
| 83 | Int. II.2 + 2-fluoro-4- methylpyri- dine-5-boronic acid | 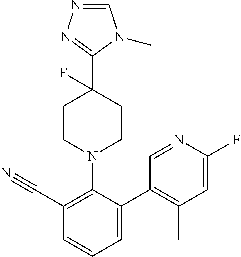 [SMILES: Cc1cc(F)ncc1-c1cccc(C#N)c1N1CCC(F)(c2nncn2C)CC1] | 1.1 eq of 2-fluoro-4-methylpyri- dine-5-boronic acid; 4.0 eq. of K₂CO₃ solution; 1 h at 70° C. |
| 85 | Int. II.2 + (4-chloro-6- fluoropyridin- 3-yl)boronic acid | 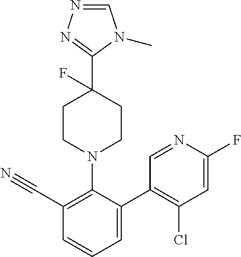 [SMILES: Cn1cnnc1C1(F)CCN(c2c(C#N)cccc2-c2cnc(F)cc2Cl)CC1] | 1 h at 70° C. |
| 86 | Int. II.2 + Int. III.20 | 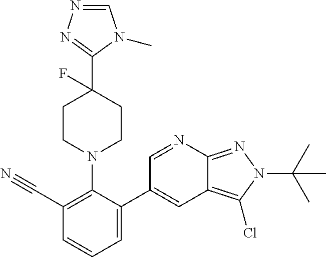 [SMILES: Cn1cnnc1C1(F)CCN(c2c(C#N)cccc2-c2cnc3nn(C(C)(C)C)c(Cl)c3c2)CC1] | 1.3 eq. of Int. III.20 was used 3 eq. of 2 M aq. Na₂CO₃ solution. |

### Example 34

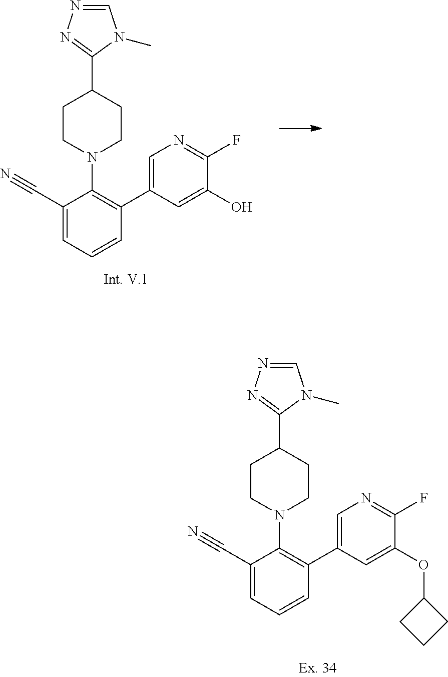 [SMILES: Cn1cnnc1C1CCN(c2c(C#N)cccc2-c2cnc(F)c(O)c2)CC1 ; Cn1cnnc1C1CCN(c2c(C#N)cccc2-c2cnc(F)c(OC3CCC3)c2)CC1]

Intermediate V.1 (60.0 mg, 159 mmol) is added to DMF (1.0 mL) and cesium carbonate (155 mg, 476 mmol) and bromocyclobutane (98.2 mg, 634 mmol) are added. The resulting reaction mixture is heated to 100° C. and stirred at this temperature for 3 h. After cooling to ambient temperature, the reaction mixture is diluted with water and MeOH and purified by preparative HPLC (XBridge C18, ACN/water gradient containing 0.1% NH₃) to yield the desired product.

C₂₄H₂₅FN₆O (M=432.5 g/mol)

ESI-MS: 433 [M+H]+

Rt (HPLC): 0.53 min (method C)

¹H NMR (400 MHz, DMSO-d6) δ=8.30 (s, 1H), 7.77 (dd, J=7.7, 1.6 Hz, 1H), 7.72 (t, J=1.8 Hz, 1H), 7.49-7.56 (m, 2H), 7.27 (t, J=7.7 Hz, 1H), 4.90 (quin, J=7.1 Hz, 1H), 3.58 (s, 3H), 3.20-3.28 (m, 2H), 2.91-3.02 (m, 2H), 2.85 (tt, J=11.2, 3.8 Hz, 1H), 2.43-2.51 (m, 2H), 2.08-2.20 (m, 2H), 1.55-1.86 (m, 6H)

The following examples were prepared with only minor modifications of the procedure of example 34:

| | Starting | | |
|---|---|---|---|
| Ex. | material | Structure | Deviation from general procedure |
| 35 | Int. V.1 + benzyl bromide | 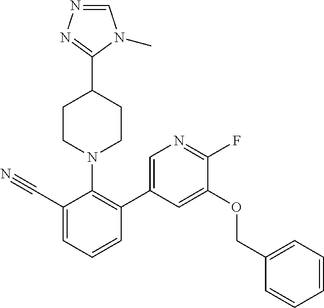 [SMILES: Cn1cnnc1C1CCN(c2c(C#N)cccc2-c2cnc(F)c(OCc3ccccc3)c2)CC1] | 2 eq. of benzyl bromide is used. |
| 36 | Int. V.1 + (iodometh- yl)cyclopro- pane | 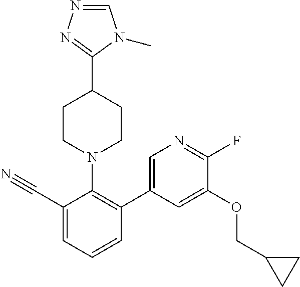 [SMILES: Cn1cnnc1C1CCN(c2c(C#N)cccc2-c2cnc(F)c(OCC3CC3)c2)CC1] | 2 eq. of (iodomethyl)cyclopropane is used; 3 h at 80° C. |
| 47 | Int. V.1 + iodocyclopro- pane | 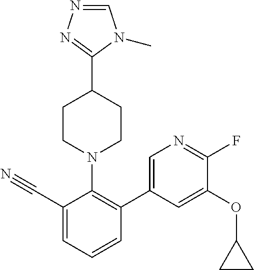 [SMILES: Cn1cnnc1C1CCN(c2c(C#N)cccc2-c2cnc(F)c(OC3CC3)c2)CC1] | 1.5 eq. of iodocyclopropane is used; 1.5 eq. of cesium carbonate is used; 16 h at 50° C. |

### Example 37

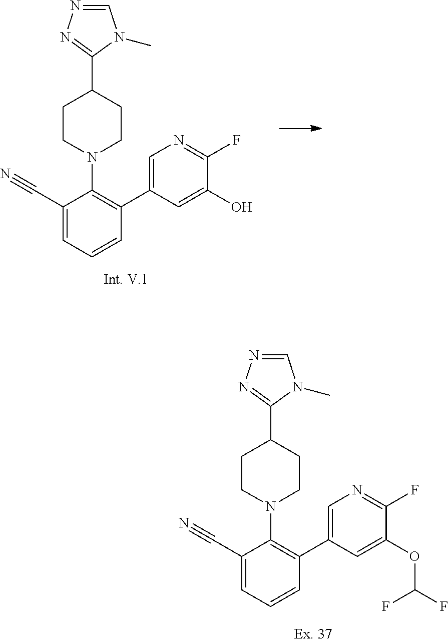 [SMILES: Cn1cnnc1C1CCN(c2c(C#N)cccc2-c2cnc(F)c(O)c2)CC1 ; Cn1cnnc1C1CCN(c2c(C#N)cccc2-c2cnc(F)c(OC(F)F)c2)CC1]

In a microwave vial, intermediate V.1 (50.0 mg, 132 μmol) and potassium carbonate (45.7 mg, 330 μmol) are added to a mixture of DMF and water (9:1, 1.5 mL) and sodium chlorodifluoroacetate (42.0 mg, 264 μmol) is added. The vial is sealed and the resulting reaction mixture is stirred at 120° C. for 3 h. After cooling to ambient temperature a second crop of sodium chlorodifluoroacetate (42.0 mg, 264 μmol) is added. The vial is sealed and the resulting reaction mixture is stirred at 120° C. for 2 h. After cooling to ambient temperature, the mixture is diluted with water and MeOH, filtered and purified by preparative HPLC (Sunfire C18, ACN/water gradient containing 0.1% TFA). C₂₁H₁₉F₃N₆O (M=428.4 g/mol)

ESI-MS: 429 [M+H]+

Rt (HPLC): 0.64 min (method H)

¹H NMR (400 MHz, DMSO-d6) δ=8.97 (s, 1H), 8.16 (t, J=1.6 Hz, 1H), 8.04 (dd, J=9.4, 1.5 Hz, 1H), 7.82 (dd, J=7.7, 1.6 Hz, 1H), 7.60 (dd, J=7.6, 1.6 Hz, 1H), 7.33 (t, J=7.7 Hz, 1H), 7.38 (t, J=72.5 Hz, 1H), 3.72 (s, 3H), 3.18-3.28 (m, 2H), 2.94-3.11 (m, 3H), 1.79-1.90 (m, 2H), 1.55-1.70 (m, 2H).

### Example 38

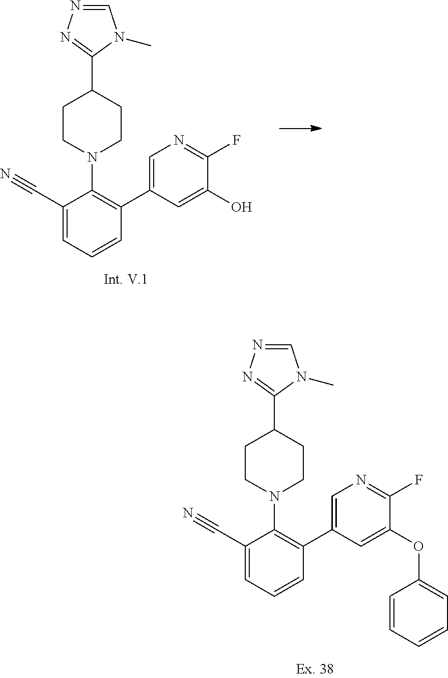 [SMILES: Cn1cnnc1C1CCN(c2c(C#N)cccc2-c2cnc(F)c(O)c2)CC1 ; Cn1cnnc1C1CCN(c2c(C#N)cccc2-c2cnc(F)c(Oc3ccccc3)c2)CC1]

In a microwave vial, intermediate V.1 (50.0 mg, 132 μmol) is added to DMSO (0.5 mL) and iodobenzene (33 mg, 159 μmol), copper(I)iodide (6.3 mg, 33 μmol), potassium phosphate monohydrate (96 mg, 396 μmol) and picolinic acid (3.3 mg, 26 μmol) are added under an argon atmosphere. The vial is sealed and the resulting reaction mixture stirred at 100° C. for 45 min and at 130° C. for 3 h. After cooling to ambient temperature, the mixture is diluted with DCM and washed with half-conc. aqueous ammonia solution. The organic phase is dried, concentrated and purified by preparative HPLC (X-Bridge C₁₈, ACN/water gradient containing 0.1% NH₃) to yield the desired product.

C₂₆H₂₃FN₆O (M=454.5 g/mol)

ESI-MS: 455 [M+H]+

Rt (HPLC): 0.86 min (method I)

¹H NMR (400 MHz, DMSO-d6) δ=8.33 (s, 1H), 8.03 (t, J=1.8 Hz, 1H), 7.76 (dd, J=7.7, 1.6 Hz, 1H), 7.59 (dd, J=9.8, 2.0 Hz, 1H), 7.55 (dd, J=7.7, 1.7 Hz, 1H), 7.39-7.46 (m, 2H), 7.27 (t, J=7.7 Hz, 1H), 7.17-7.23 (m, 3H), 3.61 (s, 3H), 3.13-3.21 (m, 2H), 2.88-2.99 (m, 2H), 2.84 (tt, J=11.4, 3.6 Hz, 1H), 1.72-1.82 (m, 2H), 1.55-1.68 (m, 2H).

### Example 41

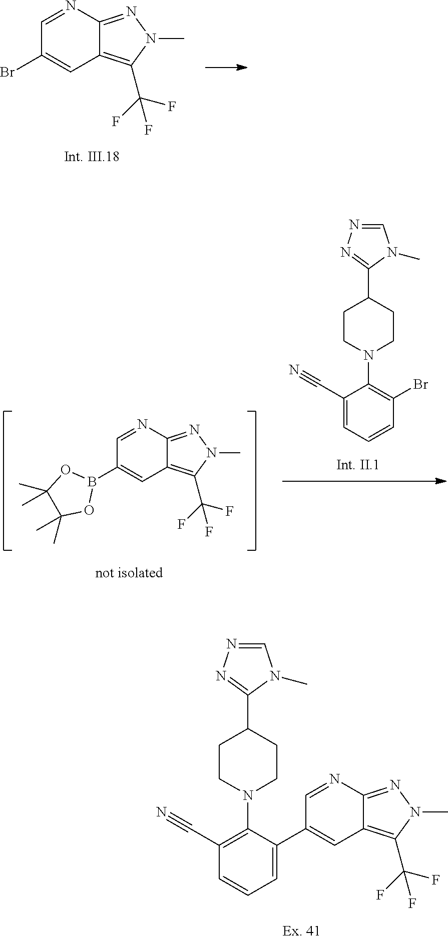

Intermediate III.18 (72.8 mg, 0.260 mmol), potassium acetate (51.0 mg, 0.52 mmol) and bis(pinacolato)diboron (66.0 mg, 0.260 mmol) are suspended in 1,4-dioxane (1.5 mL) and the resulting mixture is purged with argon for 15 min. [1,1′-Bis-(diphenylphosphino)-ferrocen]-dichloro-palladium(II) dichloride DCM complex (Pd(dppf)Cl₂*CH₂Cl₂, CAS: 95464-05-4) (14.1 mg, 0.017 mmol) is added, and the mixture is further purged with argon for 3 min. The reaction mixture is heated to 100° C. and stirred for 4 h. After being cooled to ambient temperature, intermediate 11.1 (60.0 mg, 0.173 mmol), a Na₂CO₃ solution (2M in H₂O, 260 μL, 0.520 mmol) and (Pd(dppf)Cl₂*CH₂Cl₂, CAS: 95464-05-4) (14.1 mg, 0.017 mmol) is added. The mixture is purged again with argon for 3 min and heated to and stirred at 100° C. for 4 h. After cooling to ambient temperature, the reaction is diluted with a mixture of water/ACN, acidified with TFA, filtered and purified by preparative HPLC (Sunfire C18, ACN/water gradient containing 0.1% TFA) to yield the desired compound.

C₂₃H₂₁F₃N₈ (M=466.5 g/mol)

ESI-MS: 467 [M+H]+

Rt (HPLC): 0.78 min (method D)

¹H NMR (400 MHz, DMSO-d6) δ=8.97 (s, 1H), 8.79 (d, J=2.0 Hz, 1H), 8.21-8.27 (m, 1H), 7.82 (dd, J=7.7, 1.5 Hz, 1H), 7.68 (dd, J=7.6, 1.5 Hz, 1H), 7.35 (t, J=7.7 Hz, 1H), 4.38 (s, 3H), 3.69 (s, 3H), 3.23-3.34 (m, 2H), 2.94-3.13 (m, 3H), 1.73-1.84 (m, 2H), 1.47-1.63 (m, 2H)

The following examples were prepared with only minor modifications of the procedure of example 41:

| | Starting | | |
|---|---|---|---|
| Ex. | material | Structure | Deviation from general procedure |
| 28 | Int. II.1 + 3-bromo-7-(3- chloro-phenyl)- imidazo[1,2- a]pyrimidine | 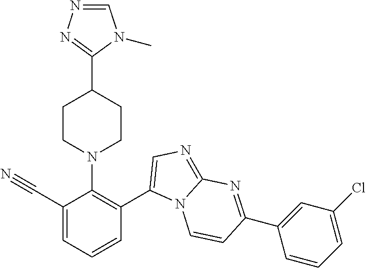 [SMILES: Cn1cnnc1C1CCN(c2c(C#N)cccc2-c2cnc3nc(-c4cccc(Cl)c4)ccn23)CC1] | 2 eq. of 3-bromo-7-(3-chloro-phe- nyl)-imidazo[1,2-a]pyrimidine; 3 eq. of bis(pinacolato)diboron; 6 eq. of potassium acetate; boryla- tion for 16 h at 70° C.; Suzuki cou- pling for 18 h at 80° C. |
| 42 | Int. II.1 + 5-bromo-3- chloro-2- fluoro- pyridine | 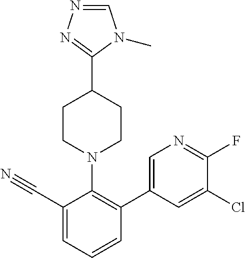 [SMILES: Cn1cnnc1C1CCN(c2c(C#N)cccc2-c2cnc(F)c(Cl)c2)CC1] | None. |
| 43 | Int. II.1 + 6-Bromo- [1,2,4]thiadia- zolo[3,4-b]pyri- dine | 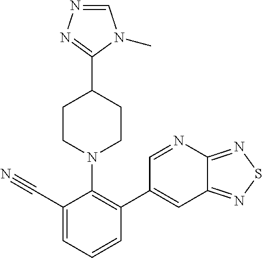 [SMILES: Cn1cnnc1C1CCN(c2c(C#N)cccc2-c2cnc3nsnc3c2)CC1] | None. |
| 44 | Int. II.1 + 5-Bromo-2- fluoro-4-meth- oxypyridine | 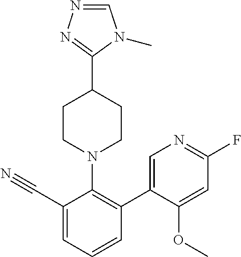 [SMILES: COc1cc(F)ncc1-c1cccc(C#N)c1N1CCC(c2nncn2C)CC1] | None. |
| 48 | Int. II.1 + 5-Chloro-3- methoxypyri- dazine | 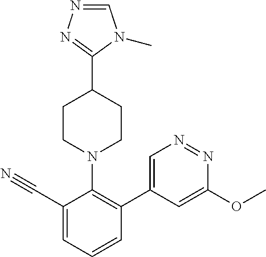 [SMILES: COc1cc(-c2cccc(C#N)c2N2CCC(c3nncn3C)CC2)cnn1] | None. |
| 57 | Int. II.1 + 3-bromopyra- zolo[1,5-b]pyri- dazine | 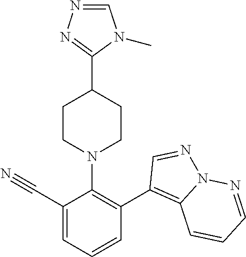 [SMILES: Cn1cnnc1C1CCN(c2c(C#N)cccc2-c2cnn3ncccc23)CC1] | 2 eq. of 3-bromopyrazolo[1,5- b]pyridazine; 3 eq. of bis(pinaco- lato)diboron; 6 eq. of potassium acetate; borylation for 16 h at 70° C.; Suzuki coupling for 18 h at 80° C. |
| 68 | Int. II.2 + 3-bromo-1,8- naphthyridine | 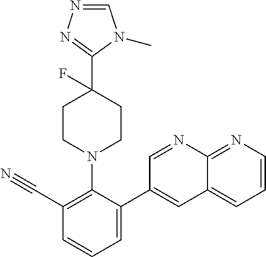 [SMILES: Cn1cnnc1C1(F)CCN(c2c(C#N)cccc2-c2cnc3ncccc3c2)CC1] | None. |
| 69 | Int. II.2 + Int. III.9 | 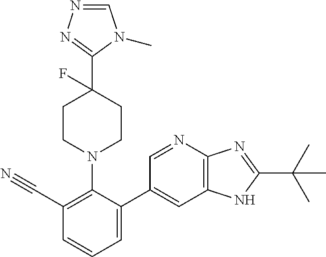 [SMILES: Cn1cnnc1C1(F)CCN(c2c(C#N)cccc2-c2cnc3nc(C(C)(C)C)[nH]c3c2)CC1] | None. |
| 76 | Int. II.1 + Int. III.17 | 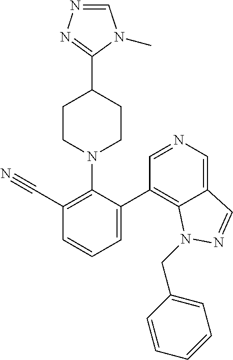 [SMILES: Cn1cnnc1C1CCN(c2c(C#N)cccc2-c2cncc3cnn(Cc4ccccc4)c23)CC1] | 1.0 eq. Int. XX; 1.3 eq. Bispinaco- lato diboron; 2 h at 100° C. for borylation; 3 eq. 2 M K₃PO₄ solu- tion; 3 h at 100° C. for Suzuki cou- pling; puridied by preparative HPLC (XBridge C18, wa- ter/MeCN gradient containing 0.1% NH₃) |
| 77 | Int. II.1 + Int. III.16 | 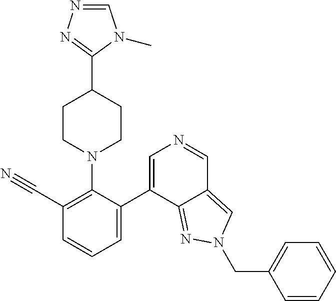 [SMILES: Cn1cnnc1C1CCN(c2c(C#N)cccc2-c2cncc3cn(Cc4ccccc4)nc23)CC1] | 1.0 eq. Int. XX; 1.3 eq. Bispinaco- lato diboron; 2 h at 100° C. for borylation; 3 eq. 2 M K₃PO₄ solu- tion; 3 h at 100° C. for Suzuki cou- pling; additionally purified by pre- parative SFC (Torus 2 PIC, MeOH/CO₂) |
| 78 | Int. II.2 + Int. III.16 | 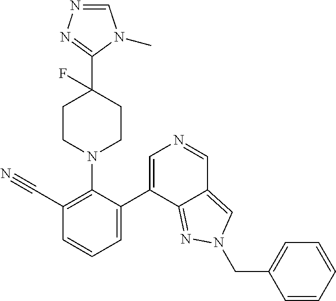 [SMILES: Cn1cnnc1C1(F)CCN(c2c(C#N)cccc2-c2cncc3cn(Cc4ccccc4)nc23)CC1] | 1.0 eq. Int. XX; 1.3 eq. Bispinaco- lato diboron; 2 h at 100° C. for borylation; 3 eq. 2 M K₃PO₄ solu- tion; 3 h at 100° C. for Suzuki cou- pling; purified by preparative HPLC (XBridge C18, wa- ter/MeCN gradient containing 0.1% NH₃) |
| 84 | Int. II.2 + 3-bromo-7-(3- chloro-phenyl)- imidazo[1,2- a]pyrimidine | 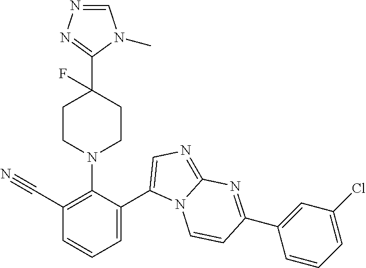 [SMILES: Cn1cnnc1C1(F)CCN(c2c(C#N)cccc2-c2cnc3nc(-c4cccc(Cl)c4)ccn23)CC1] | 1.5 eq. 1.3 eq. Bispinacolato dibo- ron; 18 h at 70° C. for borylation; 1.5 eq. 2 M K₂CO₃ solution; 3 h at 70° C. for Suzuki coupling. |

### Example 55

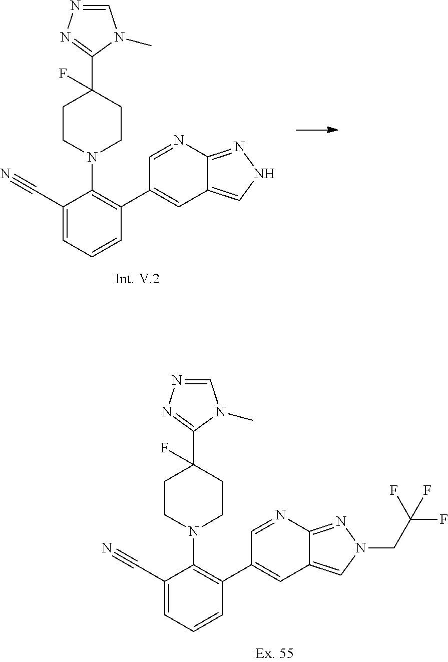 [SMILES: Cn1cnnc1C1(F)CCN(c2c(C#N)cccc2-c2cnc3n[nH]cc3c2)CC1 ; Cn1cnnc1C1(F)CCN(c2c(C#N)cccc2-c2cnc3nn(CC(F)(F)F)cc3c2)CC1]

Intermediate V.2 (20.0 mg, 49.7 μmol) is added to THE (2.0 mL) and solution of NaHMDS in THE (1.0 m, 49.7 μL, 49.7 μmol) is added. The mixture is stirred for 5 minute at ambient temperature. 2,2,2-trifluoroethyl trifluoromethanesulfonate (7.4 μL, 49.7 μmol) is added and the resulting reaction mixture is stirred for 1 h at ambient temperature. A second crop of 2,2,2-trifluoroethyl trifluoromethanesulfonate (7.4 μL, 49.7 μmol) is added and the reaction mixture is stirred for 2 h. The mixture is filtered and purified by preparative HPLC (X-Bridge C18, water/ACN gradient containing 0.1% NH₃) to yield the desired product.

C₂₃H₂₀F₄N₈ (M=484.5 g/mol)

ESI-MS: 485 [M+H]+

Rt (HPLC): 0.65 min (method J)

¹H NMR (400 MHz, DMSO-d6) δ=8.71 (d, J=2.3 Hz, 1H), 8.65 (s, 1H), 8.44 (s, 1H), 8.27 (d, J=2.3 Hz, 1H), 7.82 (dd, J=7.7, 1.6 Hz, 1H), 7.63 (dd, J=7.7, 1.6 Hz, 1H), 7.35 (t, J=7.7 Hz, 1H), 5.57 (q, J=9.0 Hz, 2H), 3.68 (d, J=1.5 Hz, 3H), 3.12-3.23 (m, 4H), 2.02-2.20 (m, 4H).

The following examples were prepared with only minor modifications of the procedure of example 55:

| | Starting | | |
|---|---|---|---|
| Ex. | material | Structure | Deviation from general procedure |
| 60 | Int. V.2 + 2,2-difluoro- ethyl trifluoro- methanesul- fonate | 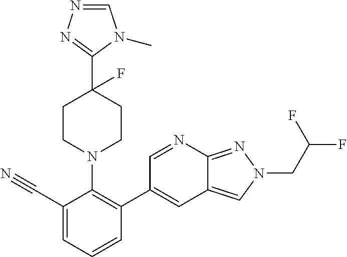 [SMILES: Cn1cnnc1C1(F)CCN(c2c(C#N)cccc2-c2cnc3nn(CC(F)F)cc3c2)CC1] | No second addition of 2,2-difluo- roethyl trifluoromethanesulfonate |

### Example 58

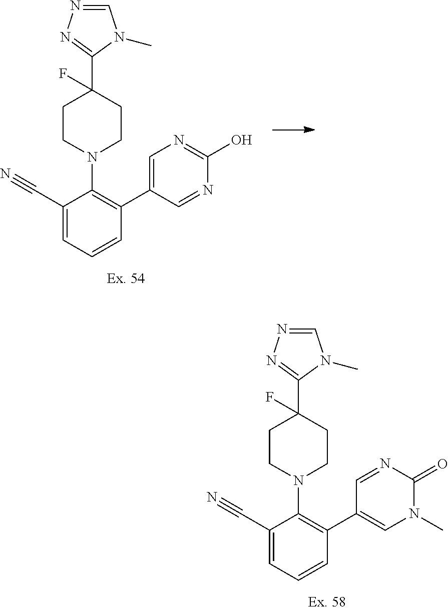 [SMILES: Cn1cnnc1C1(F)CCN(c2c(C#N)cccc2-c2cnc(O)nc2)CC1 ; Cn1cnnc1C1(F)CCN(c2c(C#N)cccc2-c2cnc(=O)n(C)c2)CC1]

Example 54 (30.0 mg, 60.8 μmol) and potassium carbonate (25.2 mg, 182 μmol) are added to DMF (2.0 mL) and iodomethane (13.0 mg, 91.2 μmol) is added. The resulting reaction mixture is stirred for 4 h at ambient temperature. It is diluted with ACN and water and directly purified by preparative HPLC (XBridge C18, ACN/water containing 0.1% TFA) to yield the desired product.

C₂₀H₂₀FN₇O (M=393.4 g/mol)

ESI-MS: 394 [M+H]+

Rt (HPLC): 0.36 min (method C)

¹H NMR (400 MHz, DMSO-d₆) δ ppm 8.73 (d, J=3.0 Hz, 1H), 8.56 (s, 1H), 8.43 (d, J=3.2 Hz, 1H), 7.78 (dd, J=7.7, 1.6 Hz, 1H), 7.63 (dd, J=7.7, 1.6 Hz, 1H), 7.36 (t, J=7.7 Hz, 1H), 3.76 (d, J=1.5 Hz, 3H), 3.51 (s, 3H), 3.33-3.45 (m, 2H), 3.13-3.22 (m, 2H), 2.08-2.29 (m, 4H)

The following examples were prepared according to the procedure of example 58:

| | Starting | | |
|---|---|---|---|
| Ex. | material | Structure | Deviation from general procedure |
| 59 | Ex. 54 + benzyl bromide | 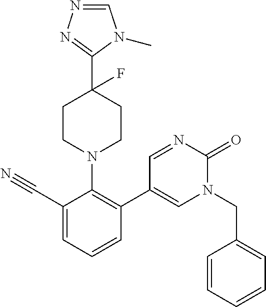 [SMILES: Cn1cnnc1C1(F)CCN(c2c(C#N)cccc2-c2cnc(=O)n(Cc3ccccc3)c2)CC1] | None. |

### Example 65

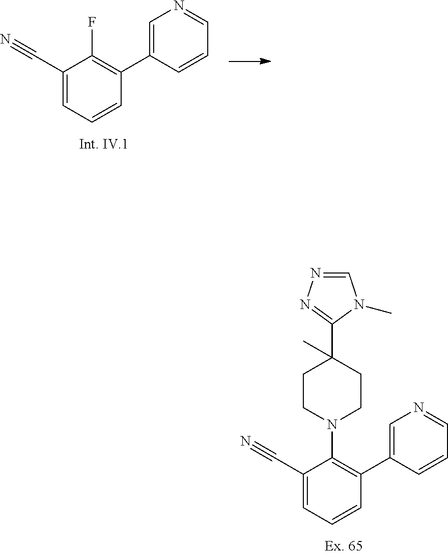 [SMILES: N#Cc1cccc(-c2cccnc2)c1F ; Cn1cnnc1C1(C)CCN(c2c(C#N)cccc2-c2cccnc2)CC1]

Intermediate IV.1 (100 mg, 0.505 mmol) is suspended in NMP (1.0 mL) and potassium carbonate (209 mg, 1.51 mmol) and 4-methyl-4-(4-methyl-1,2,4-triazol-3-yl)piperidine dihyhdrochloride (128 mg, 0.505 mmol) are added. The resulting reaction mixture is stirred at 160° C. for 18 h. After cooling to ambient temperature, the mixture is diluted with water and purified by preparative HPLC (XBridge C18, ACN/water gradient containing 0.1% NH₃) and additionally purified by preparative HPLC (XBridge C18, ACN/water gradient containing 0.1% TFA) to yield the desired product.

C₂₁H₂₂N₆ (M=358.4 g/mol)

ESI-MS: 359 [M+H]+

Rt (HPLC): 0.38 min (method K)

¹H NMR (400 MHz, DMSO-d6) δ=8.94 (s, 1H), 8.71 (d, J=1.8 Hz, 1H), 8.68 (dd, J=4.9, 1.5 Hz, 1H), 8.01 (dt, J=7.9, 1.8 Hz, 1H), 7.80 (dd, J=7.7, 1.6 Hz, 1H), 7.63 (dd, J=7.8, 5.0 Hz, 1H), 7.59 (dd, J=7.6, 1.6 Hz, 1H), 7.33 (t, J=7.7 Hz, 1H), 3.79 (s, 3H), 2.92-3.13 (m, 4H), 2.04-2.16 (m, 2H), 1.60-1.72 (m, 2H), 1.30 (s, 3H).

### Synthesis of Example 80

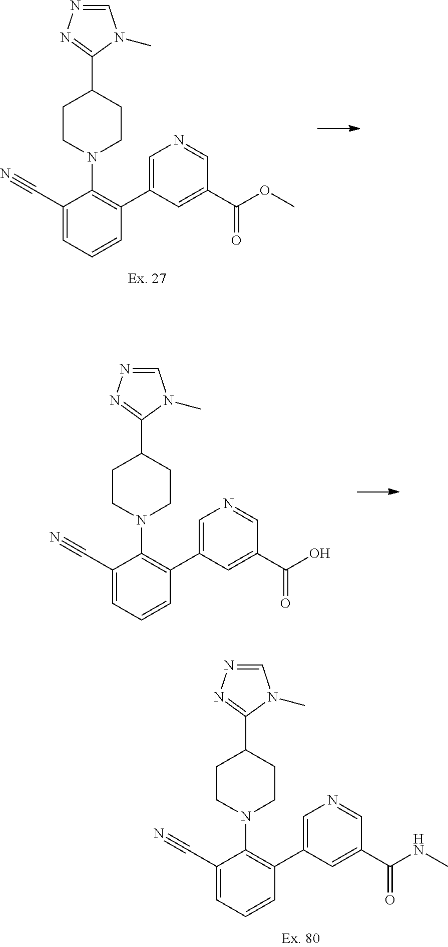 [SMILES: COC(=O)c1cncc(-c2cccc(C#N)c2N2CCC(c3nncn3C)CC2)c1 ; Cn1cnnc1C1CCN(c2c(C#N)cccc2-c2cncc(C(=O)O)c2)CC1 ; CNC(=O)c1cncc(-c2cccc(C#N)c2N2CCC(c3nncn3C)CC2)c1]

### 5-{3-Cyano-2-[4-(4-methyl-4H-1,2,4-triazol-3-yl)piperidin-1-yl]phenyl}pyridine-3-carboxylic acid

To a stirred solution of Example 27 (150 mg, 0.37 mmol) in MeOH (1.5 mL) is added 2 M aqueous solution of lithium hydroxide (0.56 mL, 1.1 mmol). After being stirred for 2 h, the reaction is neutralized with 4 M aqueous hydrochloride and concentrated. The resulting residue is suspended in H₂O and filtered to give the title compound.

C₂₁H₂₀N₆O₂ (M=388.4 g/mol)

ESI-MS: 389 [M+H]+

Rt (HPLC): 0.44 min (method H)

### Example 80

To a stirred solution of 5-{3-cyano-2-[4-(4-methyl-4H-1,2,4-triazol-3-yl)piperidin-1-yl]phenyl}pyridine-3-carboxylic acid (35 mg, 0.09 mmol) and HATU (38 mg, 0.10 mmol) in DMF (1 mL), is added DIPEA (53 μL, 0.29 mmol). After being stirred for 2 min, the reaction is treated with 2 M methylamine in MeOH (0.14 mL, 0.27 mmol) and further stirred for 18 h. Direct purification by preparative HPLC (XBridge C18, ACN/water gradient containing 0.1% NH₃) yielded Example 80.

C₂₂H₂₃N₇O (M=401.5 g/mol)

ESI-MS: 402 [M+H]+

Rt (HPLC): 0.47 min (method L)

¹H NMR (400 MHz, DMSO-d6) δ=9.00 (d, J=2.2 Hz, 1H), 8.74 (d, J=2.2 Hz, 1H), 8.67 (q, J=4.1 Hz, 1H), 8.28 (s, 1H), 8.20 (t, J=2.2 Hz, 1H), 7.80 (dd, J=7.7, 1.6 Hz, 1H), 7.59 (dd, J=7.7, 1.6 Hz, 1H), 7.32 (t, J=7.7 Hz, 1H), 3.56 (s, 3H), 3.16-3.23 (m, 2H), 2.91-3.02 (m, 2H), 2.77-2.88 (m, 4H), 1.70-1.78 (m, 2H), 1.55-1.69 (m, 2H)

### Analytical Data of Synthesized Examples

| | | Molecular Formula | |
|---|---|---|---|
| | | (MW) | |
| ex- | | ESI-MS | |
| am- | | HPLC retention time | ¹H NMR (400 MHz, |
| ple | Structure | (method) | DMSO-d6): δ in ppm |
| 2 | 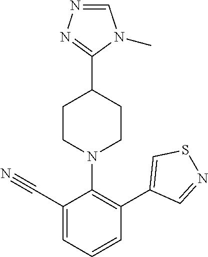 [SMILES: Cn1cnnc1C1CCN(c2c(C#N)cccc2-c2cnsc2)CC1] | C₁₈H₁₈N₆S (M = 350.4 g/mol) ESI-MS: 351 [M + H]+ Rt (HPLC): 0.60 min (method L) | 9.16 (s, 1 H), 9.07 (s, 1 H), 8.75 (s, 1 H), 7.77 (dd, J = 7.7, 1.6 Hz, 1 H), 7.63 (dd, J = 7.7, 1.6 Hz, 1 H), 7.30 (t, J = 7.7 Hz, 1 H), 3.76 (s, 3 H), 3.16 - 3.24 (m, 2 H), 3.03-3.14 (m, 3 H), 1.83-1.90 (m, 2 H), 1.64-1.77 (m, 2 H) |
| 3 | 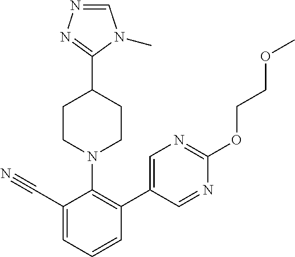 [SMILES: COCCOc1ncc(-c2cccc(C#N)c2N2CCC(c3nncn3C)CC2)cn1] | C₂₂H₂₅N₇O₂ (M = 419.5 g/mol) ESI-MS: 420 [M + H]+ Rt (HPLC): 0.59 min (method L) | 8.94 (s, 1 H), 8.68 (s, 2 H), 7.80 (dd, J = 7.7, 1.5 Hz, 1 H), 7.61 (dd, J = 7.7, 1.5 Hz, 1 H), 7.33 (t, J = 7.7 Hz, 1 H), 4.43-4.52 (m, 2 H), 3.72 (s, 3 H), 3.68-3.72 (m, 2 H), 3.31 (s, 3 H), 3.18- 3.25 (m, 2 H), 3.00-3.12 (m, 3 H), 1.81-1.89 (m, 2 H), 1.57-1.71 (m, 2 H) |
| 4 | 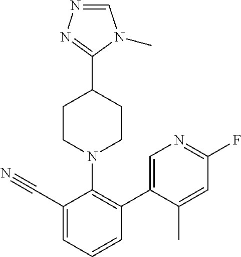 [SMILES: Cc1cc(F)ncc1-c1cccc(C#N)c1N1CCC(c2nncn2C)CC1] | C₂₁H₂₁FN₆ (M = 376.4 g/mol) ESI-MS: 377 [M+H]+ Rt (HPLC): 0.67 min (method O) | 8.28 (s, 1 H), 8.02 (s, 1 H), 7.80 (dd, J = 7.7, 1.6 Hz, 1 H), 7.45 (dd, J = 7.6, 1.6 Hz, 1 H), 7.27-7.32 (m, 1 H), 7.23 (br s, 1 H), 3.56 (s, 3 H), 3.27-3.31 (m, 1 H), 3.02-3.15 (m, 2 H), 2.71- 2.85 (m, 2 H), 2.18 (s, 3 H), 1.74-1.82 (m, 1 H), 1.54- 1.70 (m, 2 H), 1.33-1.45 (m, 1 H) |
| 5 | 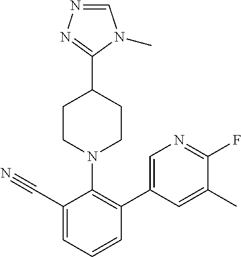 [SMILES: Cc1cc(-c2cccc(C#N)c2N2CCC(c3nncn3C)CC2)cnc1F] | C₂₁H₂₁FN₆ (M = 376.4 g/mol) ESI-MS: 377 [M + H]+ Rt (HPLC): 0.69 min (method O) | 8.30 (s, 1 H), 8.06 (br s, 1 H), 7.93 (dd, J = 9.6, 1.6 Hz, 1 H), 7.77 (dd, J = 7.7, 1.6 Hz, 1 H), 7.54 (dd, J = 7.7, 1.6 Hz, 1 H), 7.28 (t, J = 7.7 Hz, 1 H), 3.58 (s, 3 H), 3.15- 3.21 (m, 2 H), 2.93-3.04 (m, 2 H), 2.85 (tt, J = 11.3, 4.0 Hz, 1 H), 2.32 (s, 3 H), 1.72-1.80 (m, 2 H), 1.59- 1.71 (m, 2 H) |
| 6 | 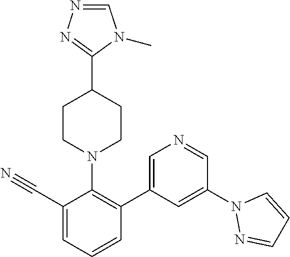 [SMILES: Cn1cnnc1C1CCN(c2c(C#N)cccc2-c2cncc(-n3cccn3)c2)CC1] | C₂₃H₂₂N₈ (M = 410.5 g/mol) ESI-MS: 411 [M+H]+ Rt (HPLC): 0.55 min (method H) | 9.16 (d, J = 2.4 Hz, 1 H), 9.12 (br s, 1 H), 8.67 (d, J = 2.5 Hz, 1 H), 8.55 (d, J = 1.6 Hz, 1 H), 8.32 (t, J = 2.1 Hz, 1 H), 7.83-7.86 (m, 1 H), 7.82 (d, J = 1.5 Hz, 1 H), 7.66 (dd, J = 7.6, 1.5 Hz, 1 H), 7.34 (t, J = 7.7 Hz, 1 H), 6.61-6.65 (m, 1 H), 3.73 (s, 3 H), 3.24-3.33 (m, 2 H), 2.94-3.12 (m, 3 H), 1.81-1.90 (m, 2 H), 1.61-1.74 (m, 2 H) |
| 7 | 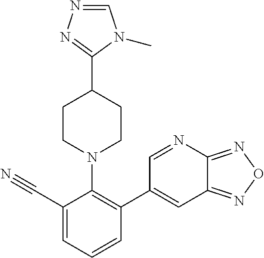 [SMILES: Cn1cnnc1C1CCN(c2c(C#N)cccc2-c2cnc3nonc3c2)CC1] | C₂₀H₁₈N₈O (M = 386.4 g/mol) ESI-MS: 387 [M + H]+ Rt (HPLC): 0.62 min (method O) | 9.12 (d, J = 2.0 Hz, 1 H), 8.58 (d, J = 2.0 Hz, 1 H), 8.28 (s, 1 H), 7.92 (dd, J = 7.8, 1.6 Hz, 1 H), 7.81 (dd, J = 7.7, 1.6 Hz, 1 H), 7.44 (t, J = 7.7 Hz, 1 H), 3.56 (s, 3 H), 3.19-3.28 (m, 4 H), 2.88 (tt, J = 11.4, 3.3 Hz, 1 H), 1.71-1.79 (m, 2 H), 1.43-1.57 (m, 2 H) |
| 8 | 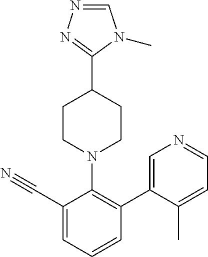 [SMILES: Cc1ccncc1-c1cccc(C#N)c1N1CCC(c2nncn2C)CC1] | C₂₁H₂₂N₆ (M = 358.4 g/mol) ESI-MS: 359 [M + H]+ Rt (HPLC): 0.38 min (method H) | 8.46 (d, J = 5.1 Hz, 1 H), 8.31 (s, 1 H), 8.27 (s, 1 H), 7.78 (dd, J = 7.7, 1.6 Hz, 1 H), 7.44 (dd, J = 7.6, 1.6 Hz, 1 H), 7.38 (d, J = 5.1 Hz, 1 H), 7.25-7.31 (m, 1 H), 3.54 (s, 3 H), 3.27-3.31 (m, 1 H), 3.01-3.13 (m, 2 H), 2.68-2.82 (m, 2 H), 2.15 (s, 3 H), 1.71-1.80 (m, 1 H), 1.53-1.67 (m, 2 H), 1.34-1.46 (m, 1 H) |
| 9 | 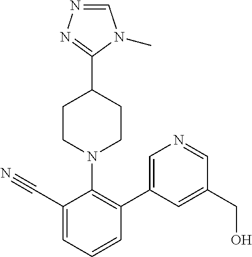 [SMILES: Cn1cnnc1C1CCN(c2c(C#N)cccc2-c2cncc(CO)c2)CC1] | C₂₁H₂₂N₆O (M = 374.4 g/mol) ESI-MS: 375 [M + H]+ Rt (HPLC): 0.36 min (method H) | 8.54 (d, J = 2.0 Hz, 1 H), 8.48 (d, J = 2.2 Hz, 1 H), 8.30 (s, 1 H), 7.74-7.80 (m, 2 H), 7.53 (dd, J = 7.6, 1.6 Hz, 1 H), 7.29 (t, J = 7.7 Hz, 1 H), 5.48 (br s, 1 H), 4.63 (s, 2 H), 3.57 (s, 3 H), 3.14-3.21 (m, 2 H), 2.92- 3.02 (m, 2 H), 2.78-2.87 (m, 1 H), 1.60-1.76 (m, 4 H) |
| 10 | 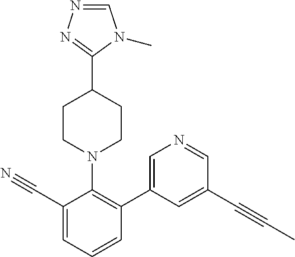 [SMILES: CC#Cc1cncc(-c2cccc(C#N)c2N2CCC(c3nncn3C)CC2)c1] | C₂₃H₂₂N₆ (M = 382.5 g/mol) ESI-MS: 383 [M + H]+ Rt (HPLC): 0.70 min (method O) | 8.59 (d, J = 1.9 Hz, 1 H), 8.52 (d, J = 2.0 Hz, 1 H), 8.29 (s, 1 H), 7.84 (t, J = 2.0 Hz, 1 H), 7.78 (dd, J = 7.7, 1.6 Hz, 1 H), 7.55 (dd, J = 7.7, 1.6 Hz, 1 H), 7.28 (t, J = 7.7 Hz, 1 H), 3.57 (s, 3 H), 3.16-3.23 (m, 2 H), 2.89-3.00 (m, 2 H), 2.78- 2.87 (m, 1 H), 2.10 (s, 3 H), 1.72-1.79 (m, 2 H), 1.59- 1.70 (m, 2 H) |
| 11 | 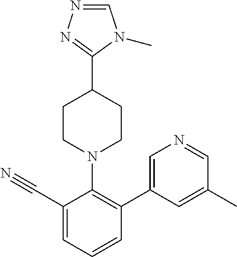 [SMILES: Cc1cncc(-c2cccc(C#N)c2N2CCC(c3nncn3C)CC2)c1] | C₂₁H₂₂N₆ (M = 358.4 g/mol) ESI-MS: 359 [M + H]+ Rt (HPLC): 0.39 min (method H) | 9.06 (s, 1 H), 8.59 (s, 2 H), 8.00 (br s, 1 H), 7.82 (dd, J = 7.7, 1.6 Hz, 1 H), 7.60 (dd, J = 7.6, 1.5 Hz, 1 H), 7.34 (t, J = 7.7 Hz, 1 H), 3.73 (s, 3 H), 3.17-3.24 (m, 2 H), 2.96-3.11 (m, 3 H), 2.45 (s, 3 H), 1.78- 1.87 (m, 2 H), 1.52-1.67 (m, 2 H) |
| 12 | 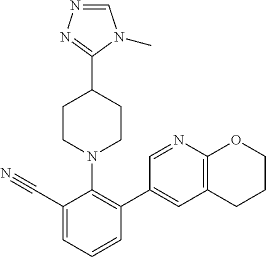 [SMILES: Cn1cnnc1C1CCN(c2c(C#N)cccc2-c2cnc3c(c2)CCCO3)CC1] | C₂₃H₂₄N₆O (M = 400.5 g/mol) ESI-MS: 401 [M+H]+ Rt (HPLC): 0.48 min (method H) | 8.30 (s, 1 H), 7.99 (d, J = 2.4 Hz, 1 H), 7.71 (dd, J = 7.7, 1.6 Hz, 1 H), 7.59 (d, J = 2.2 Hz, 1 H), 7.49 (dd, J = 7.6, 1.6 Hz, 1 H), 7.24 (t, J = 7.7 Hz, 1 H), 4.28-4.34 (m, 2 H), 3.59 (s, 3 H), 3.15- 3.22 (m, 2 H), 2.93-3.03 (m, 2 H), 2.82-2.91 (m, 3 H), 1.91-2.00 (m, 2 H), 1.64-1.80 (m, 4 H) |
| 13 | 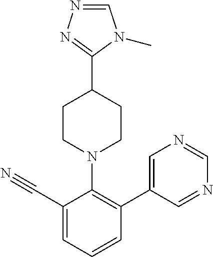 [SMILES: Cn1cnnc1C1CCN(c2c(C#N)cccc2-c2cncnc2)CC1] | C₁₉H₁₉N₇ (M = 345.4 g/mol) ESI-MS: 346 [M + H]+ Rt (HPLC): 0.35 min (method C) | 9.22 (s, 1 H), 9.04 (s, 1 H), 8.90 (s, 2 H), 7.84 (dd, J = 7.7, 1.5 Hz, 1 H), 7.66 (dd, J = 7.7, 1.6 Hz, 1 H), 7.37 (t, J = 7.7 Hz, 1 H), 3.73 (s, 3 H), 3.17-3.25 (m, 2 H), 3.01-3.13 (m, 3 H), 1.79-1.88 (m, 2 H), 1.52-1.66 (m, 2 H) |
| 14 | 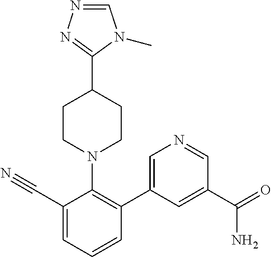 [SMILES: Cn1cnnc1C1CCN(c2c(C#N)cccc2-c2cncc(C(N)=O)c2)CC1] | C₂₁H₂₁N₇O (M = 387.4 g/mol) ESI-MS: 388 [M+H]+ Rt (HPLC): 0.42 min (method H) | 9.04 (d, J = 2.0 Hz, 1 H), 8.75 (d, J = 2.0 Hz, 1 H), 8.28 (s, 1 H), 8.24 (t, J = 2.1 Hz, 1 H), 8.19 (br s, 1 H), 7.80 (dd, J = 7.7, 1.6 Hz, 1 H), 7.64 (br s, 1 H), 7.60 (dd, J = 7.7, 1.6 Hz, 1 H), 7.32 (t, J = 7.7 Hz, 1 H), 3.56 (s, 3 H), 3.16-3.23 (m, 2 H), 2.90-3.01 (m, 2 H), 2.77-2.86 (m, 1 H), 1.70-1.79 (m, 2 H), 1.56- 1.69 (m, 2 H) |
| 15 | 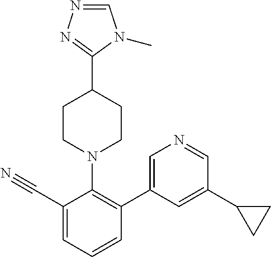 [SMILES: Cn1cnnc1C1CCN(c2c(C#N)cccc2-c2cncc(C3CC3)c2)CC1] | C₂₃H₂₄N₆ (M = 384.5 g/mol) ESI-MS: 385 [M + H]+ Rt (HPLC): 0.44 min (method H) | 9.19 (s, 1 H), 8.61 (d, J = 2.0 Hz, 1 H), 8.58 (d, J = 1.1 Hz, 1 H), 7.79 - 7.86 (m, 2 H), 7.60 (dd, J = 7.7, 1.6 Hz, 1 H), 7.34 (t, J = 7.7 Hz, 1 H), 3.76 (s, 3 H), 3.17-3.26 (m, 2 H), 2.94-3.12 (m, 3 H), 2.08-2.17 (m, 1 H), 1.79-1.90 (m, 2 H), 1.50- 1.66 (m, 2 H), 1.05-1.13 (m, 2 H), 0.90-0.96 (m, 2 H) |
| 16 | 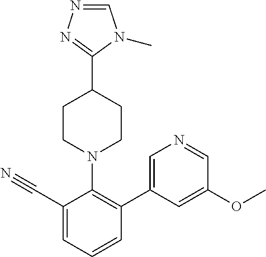 [SMILES: COc1cncc(-c2cccc(C#N)c2N2CCC(c3nncn3C)CC2)c1] | C₂₁H₂₂N₆O (M = 374.4 g/mol) ESI-MS: 375 [M+H]+ Rt (HPLC): 0.61 min (method O) | 9.03 (s, 1 H), 8.35 (d, J=2.8 Hz, 1 H), 8.22 (d, J = 1.5 Hz, 1 H), 7.79 (dd, J = 7.7, 1.5 Hz, 1 H), 7.56 (dd, J = 7.6, 1.5 Hz, 1 H), 7.49-7.53 (m, 1 H), 7.26-7.33 (m, 1 H), 3.91 (s, 3 H), 3.73 (s, 3 H), 3.20-3.28 (m, 2 H), 3.06 (tt, J = 11.7, 3.6 Hz, 1 H), 2.90-3.01 (m, 2 H), 1.80-1.88 (m, 2 H), 1.60- 1.74 (m, 2 H) |
| 17 | 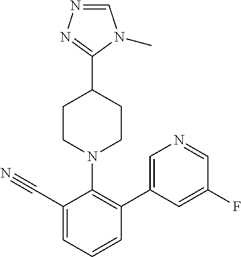 [SMILES: Cn1cnnc1C1CCN(c2c(C#N)cccc2-c2cncc(F)c2)CC1] | C₂₀H₁₉FN₆ (M = 362.4 g/mol) ESI-MS: 363 [M + H]+ Rt (HPLC): 0.62 min (method O) | 9.11 (s, 1 H), 8.63 (d, J=2.7 Hz, 1 H), 8.46-8.51 (m, 1 H), 7.80-7.88 (m, 2 H), 7.59 (dd, J = 7.7, 1.6 Hz, 1 H), 7.32 (t, J = 7.7 Hz, 1 H), 3.75 (s, 3 H), 3.19-3.27 (m, 2 H), 3.08 (tt, J = 11.8, 3.4 Hz, 1 H), 2.92-3.03 (m, 2 H), 1.80-1.88 (m, 2 H), 1.56-1.70 (m, 2 H) |
| 18 | 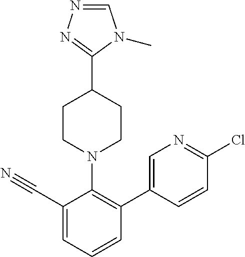 [SMILES: Cn1cnnc1C1CCN(c2c(C#N)cccc2-c2ccc(Cl)nc2)CC1] | C₂₀H₁₉ClN₆ (M = 378.9 g/mol) ESI-MS: 379 [M+H]+ Rt (HPLC): 0.60 min (method H) | 8.46 (d, J = 2.2 Hz, 1 H), 8.30 (s, 1 H), 7.93 (dd, J = 8.2, 2.5 Hz, 1 H), 7.79 (dd, J = 7.7, 1.6 Hz, 1 H), 7.63 (dd, J = 8.2, 0.5 Hz, 1 H), 7.55 (dd, J = 7.7, 1.6 Hz, 1 H), 7.30 (t, J = 7.7 Hz, 1 H), 3.58 (s, 3 H), 3.16- 3.23 (m, 2 H), 2.92-3.03 (m, 2 H), 2.85 (tt, J = 11.5, 3.8 Hz, 1 H), 1.73-1.81 (m, 2 H), 1.60-1.73 (m, 2 H) |
| 19 | 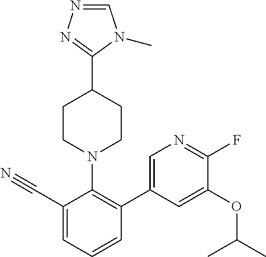 [SMILES: CC(C)Oc1cc(-c2cccc(C#N)c2N2CCC(c3nncn3C)CC2)cnc1F] | C₂₃H₂₅FN₆O (M = 420.5 g/mol) ESI-MS: 421 [M + H]+ Rt (HPLC): 0.78 min (method O) | 8.29 (s, 1 H), 7.78 (dd, J = 7.7, 1.6 Hz, 1 H), 7.70- 7.76 (m, 2 H), 7.55 (dd, J = 7.7, 1.6 Hz, 1 H), 7.27 (t, J = 7.7 Hz, 1 H), 4.80 (spt, J = 6.0 Hz, 1 H), 3.57 (s, 3 H), 3.19-3.28 (m, 2 H), 2.91-3.04 (m, 2 H), 2.84 (tt, J = 11.3, 3.7 Hz, 1 H), 1.60-1.83 (m, 4 H), 1.32 (d, J = 6.0 Hz, 6 H) |
| 20 | 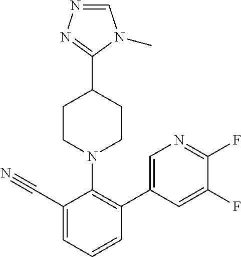 [SMILES: Cn1cnnc1C1CCN(c2c(C#N)cccc2-c2cnc(F)c(F)c2)CC1] | C₂₀H₁₈F₂N₆ (M = 380.4 g/mol) ESI-MS: 381 [M + H]+ Rt (HPLC): 0.69 min (method O) | 8.30 (s, 1 H), 8.15-8.24 (m, 1 H), 8.11 (t, J = 1.6 Hz, 1 H), 7.81 (dd, J = 7.7, 1.5 Hz, 1 H), 7.59 (dd, J = 7.7, 1.6 Hz, 1 H), 7.31 (t, J = 7.7 Hz, 1 H), 3.58 (s, 3 H), 3.16- 3.25 (m, 2 H), 2.95-3.07 (m, 2 H), 2.87 (tt, J = 11.5, 3.7 Hz, 1 H), 1.73-1.82 (m, 2 H), 1.58-1.71 (m, 2 H) |
| 21 | 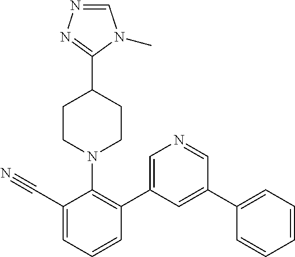 [SMILES: Cn1cnnc1C1CCN(c2c(C#N)cccc2-c2cncc(-c3ccccc3)c2)CC1] | C₂₆H₂₄N₆ (M = 420.5 g/mol) ESI-MS: 421 [M+H]+ Rt (HPLC): 0.78 min (method O) | 8.91 (d, J = 2.2 Hz, 1 H), 8.60 (d, J = 1.9 Hz, 1 H), 8.28 (s, 1 H), 8.12 (t, J = 2.1 Hz, 1 H), 7.82-7.86 (m, 2 H), 7.79 (dd, J = 7.7, 1.6 Hz, 1 H), 7.64 (dd, J = 7.7, 1.6 Hz, 1 H), 7.49-7.56 (m, 2 H), 7.41-7.47 (m, 1 H), 7.31 (t, J = 7.7 Hz, 1 H), 3.55 (s, 3 H), 3.24-3.30 (m, 2 H), 2.95-3.06 (m, 2 H), 2.80-2.90 (m, 1 H), 1.63-1.81 (m, 4 H) |
| 22 | 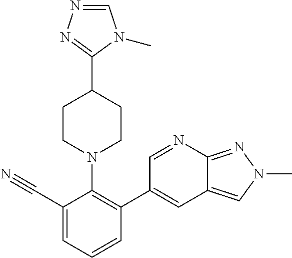 [SMILES: Cn1cc2cc(-c3cccc(C#N)c3N3CCC(c4nncn4C)CC3)cnc2n1] | C₂₂H₂₂N₈ (M = 398.5 g/mol) ESI-MS: 399 [M + H]+ Rt (HPLC): 0.40 min (method C) | 9.04 (s, 1 H), 8.59 (d, J = 2.0 Hz, 1 H), 8.48 (s, 1 H), 8.19 (d, J = 2.2 Hz, 1 H), 7.78 (dd, J = 7.7, 1.6 Hz, 1 H), 7.59 (dd, J = 7.6, 1.5 Hz, 1 H), 7.31 (t, J = 7.7 Hz, 1 H), 4.23 (s, 3 H), 3.70 (s, 3 H), 3.21-3.29 (m, 2 H), 2.89- 3.06 (m, 3 H), 1.74-1.83 (m, 2 H), 1.55-1.69 (m, 2 H) |
| 23 | 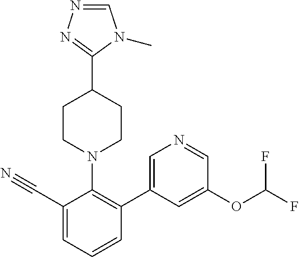 [SMILES: Cn1cnnc1C1CCN(c2c(C#N)cccc2-c2cncc(OC(F)F)c2)CC1] | C₂₁H₂₀F₂N₆O (M = 410.4 g/mol) ESI-MS: 411 [M+H]+ Rt (HPLC): 0.47 min (method C) | 9.07 (s, 1 H), 8.53 (d, J = 2.5 Hz, 1 H), 8.51 (d, J = 1.8 Hz, 1 H), 7.82 (dd, J = 7.7, 1.6 Hz, 1 H), 7.75 (t, J = 2.1 Hz, 1 H), 7.59 (dd, J = 7.6, 1.6 Hz, 1 H), 7.39 (t, J = 73.3 Hz, 1 H), 7.33 (t, J = 7.7 Hz, 1 H), 3.74 (s, 3 H), 3.18- 3.27 (m, 2 H), 2.91-3.11 (m, 3 H), 1.80-1.88 (m, 2 H), 1.56-1.71 (m, 2 H) |
| 24 | 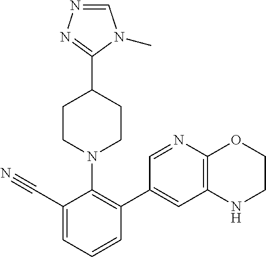 [SMILES: Cn1cnnc1C1CCN(c2c(C#N)cccc2-c2cnc3c(c2)NCCO3)CC1] | C₂₂H₂₃N₇O (M = 401.5 g/mol) ESI-MS: 402 [M + H]+ Rt (HPLC): 0.56 min (method O) | 8.30 (s, 1 H), 7.70 (dd, J = 7.6, 1.6 Hz, 1 H), 7.41 (dd, J = 7.6, 1.6 Hz, 1 H), 7.34 (d, J = 2.2 Hz, 1 H), 7.20 (t, J = 7.7 Hz, 1 H), 6.89 (d, J = 2.0 Hz, 1 H), 6.12-6.19 (m, 1 H), 4.26- 4.36 (m, 2 H), 3.59 (s, 3 H), 3.31-3.34 (m, 2 H), 3.17- 3.27 (m, 2 H), 2.76-2.97 (m, 3 H), 1.75-1.87 (m, 4 H) |
| 25 | 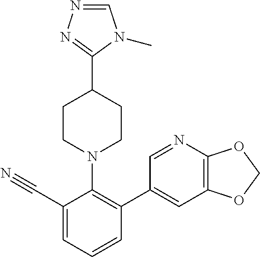 [SMILES: Cn1cnnc1C1CCN(c2c(C#N)cccc2-c2cnc3c(c2)OCO3)CC1] | C₂₁H₂₀N₆O₂ (M = 388.4 g/mol) ESI-MS: 389 [M+H]+ Rt (HPLC): 0.56 min (method M) | 8.98 (s, 1 H), 7.75 (dd, J = 7.7, 1.6 Hz, 1 H), 7.58 (d, J = 1.8 Hz, 1 H), 7.48 (dd, J = 7.6, 1.6 Hz, 1 H), 7.31 (d, J = 1.8 Hz, 1 H), 7.25 (t, J = 7.7 Hz, 1 H), 6.21 (s, 2 H), 3.74 (s, 3 H), 3.20-3.29 (m, 2 H), 3.05 (tt, J = 11.6, 3.4 Hz, 1 H), 2.95 (br t, J = 11.3 Hz, 2 H), 1.82-1.91 (m, 2 H), 1.66- 1.80 (m, 2 H) |
| 26 | 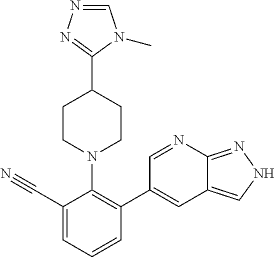 [SMILES: Cn1cnnc1C1CCN(c2c(C#N)cccc2-c2cnc3n[nH]cc3c2)CC1] | C₂₁H₂₀N₈ (M = 384.4 g/mol) ESI-MS: 385 [M + H]+ Rt (HPLC): 0.56 min (method H) | 13.74 (br s, 1 H), 8.99 (s, 1 H), 8.54 (d, J = 2.0 Hz, 1 H), 8.24 (d, J = 2.0 Hz, 1 H), 8.21 (s, 1 H), 7.79 (dd, J = 7.7, 1.6 Hz, 1 H), 7.59 (dd, J = 7.6, 1.5 Hz, 1 H), 7.31 (t, J = 7.7 Hz, 1 H), 3.69 (s, 3 H), 3.18-3.28 (m, 2 H), 2.86-3.06 (m, 3 H), 1.74-1.82 (m, 2 H), 1.55 - 1.70 (m, 2 H) |
| 27 | 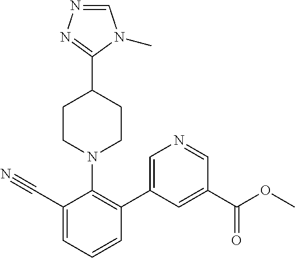 [SMILES: COC(=O)c1cncc(-c2cccc(C#N)c2N2CCC(c3nncn3C)CC2)c1] | C₂₂H₂₂N₆O₂ (M = 402.5 g/mol) ESI-MS: 403 [M + H]+ Rt (HPLC): 0.71 min (method D) | 9.10 (d, J = 2.0 Hz, 1 H), 8.87 (d, J = 2.2 Hz, 1 H), 8.36 (t, J = 2.0 Hz, 1 H), 8.28 (s, 1 H), 7.81 (dd, J = 7.7, 1.6 Hz, 1 H), 7.63 (dd, J = 7.6, 1.5 Hz, 1 H), 7.33 (t, J = 7.7 Hz, 1 H), 3.92 (s, 3 H), 3.57 (s, 3 H), 3.15-3.24 (m, 2 H), 2.94- 3.09 (m, 2 H), 2.83 (tt, J = 11.4, 3.7 Hz, 1 H), 1.68- 1.78 (m, 2 H), 1.52-1.67 (m, 2 H) |
| 28 | 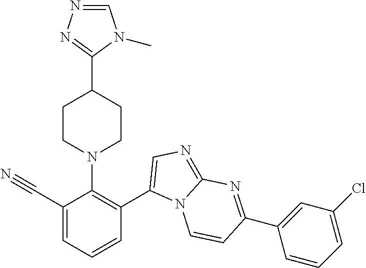 [SMILES: Cn1cnnc1C1CCN(c2c(C#N)cccc2-c2cnc3nc(-c4cccc(Cl)c4)ccn23)CC1] | C₂₇H₂₃ClN₈ (M = 495.0 g/mol) ESI-MS: 495 [M + H]+ Rt (HPLC): 0.50 min (method P) | 8.68 (d, J = 7.2 Hz, 1 H), 8.28-8.31 (m, 1 H), 8.21- 8.25 (m, 1 H), 8.20 (s, 1 H), 7.93 (s, 1 H), 7.88 (dd, J = 7.7, 1.6 Hz, 1 H), 7.76 (d, J = 7.2 Hz, 1 H), 7.73 (dd, J = 7.6, 1.5 Hz, 1 H), 7.58-7.63 (m, 2 H), 7.34 (t, J = 7.7 Hz, 1 H), 3.47 (s, 3 H), 3.34-3.38 (m, 2 H), 2.94-3.04 (m, 2 H), 2.72- 2.82 (m, 1 H), 1.59-1.67 (m, 2 H), 1.28-1.40 (m, 2 H) |
| 29 | 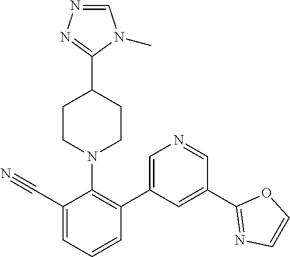 [SMILES: Cn1cnnc1C1CCN(c2c(C#N)cccc2-c2cncc(-c3ncco3)c2)CC1] | C₂₃H₂₁N₇O (M = 411.5 g/mol) ESI-MS: 412 [M + H]+ Rt (HPLC): 0.41 min (method C) | 9.19 (d, J = 2.0 Hz, 1 H), 8.88 (s, 1 H), 8.75 (d, J = 1.9 Hz, 1 H), 8.36 (t, J = 1.8 Hz, 1 H), 8.33 (s, 1 H), 7.83 (dd, J = 7.7, 1.6 Hz, 1 H), 7.66 (dd, J = 7.6, 1.5 Hz, 1 H), 7.48 (s, 1 H), 7.34 (t, J = 7.7 Hz, 1 H), 3.68 (s, 3 H), 3.22-3.31 (m, 2 H), 2.90-3.13 (m, 3 H), 1.81 (br d, J = 10.6 Hz, 2 H), 1.57- 1.72 (m, 2 H) |
| 30 | 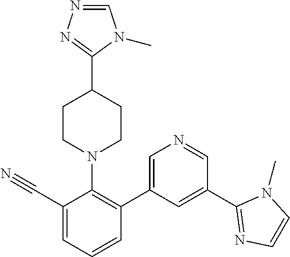 [SMILES: Cn1ccnc1-c1cncc(-c2cccc(C#N)c2N2CCC(c3nncn3C)CC2)c1] | C₂₄H₂₄N₈ (M = 424.5 g/mol) ESI-MS: 425 [M + H]+ Rt (HPLC): 0.54 min (method J) | 8.90 (d, J = 2.0 Hz, 1 H), 8.61 (d, J = 1.9 Hz, 1 H), 8.27 (s, 1 H), 8.08 (t, J = 2.0 Hz, 1 H), 7.78 (dd, J=7.7, 1.5 Hz, 1 H), 7.59 (dd, J = 7.6, 1.5 Hz, 1 H), 7.27- 7.34 (m, 2 H), 7.04 (d, J = 0.8 Hz, 1 H), 3.83 (s, 3 H), 3.54 (br s, 3 H), 3.19- 3.28 (m, 2 H), 2.92-3.04 (m, 2 H), 2.76-2.87 (m, 1 H), 1.71-1.80 (m, 2 H), 1.57-1.70 (m, 2 H) |
| 31 | 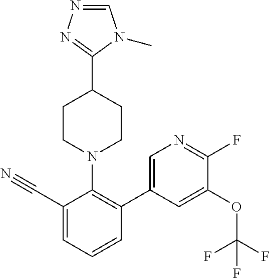 [SMILES: Cn1cnnc1C1CCN(c2c(C#N)cccc2-c2cnc(F)c(OC(F)(F)F)c2)CC1] | C₂₁H₁₈F₄N₆O (M = 446.4 g/mol) ESI-MS: 447 [M + H]+ Rt (HPLC): 0.52 min (method C) | 8.80 (s, 1 H), 8.30-8.36 (m, 2 H), 7.84 (dd, J = 7.7, 1.6 Hz, 1 H), 7.63 (dd, J = 7.6, 1.6 Hz, 1 H), 7.35 (t, J = 7.7 Hz, 1 H), 3.68 (s, 3 H), 3.18-3.27 (m, 2 H), 2.91-3.10 (m, 3 H), 1.77- 1.89 (m, 2 H), 1.51-1.65 (m, 2 H) |
| 32 | 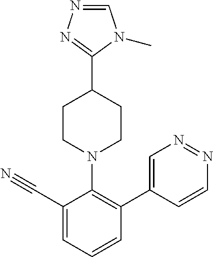 [SMILES: Cn1cnnc1C1CCN(c2c(C#N)cccc2-c2ccnnc2)CC1] | C₁₉H₁₉N₇ (M = 345.4 g/mol) ESI-MS: 346 [M + H]+ Rt (HPLC): 0.60 min (method D) | 9.33-9.36 (m, 2 H), 9.14 (s, 1 H), 7.87 (dd, J = 7.7, 1.6 Hz, 1 H), 7.76-7.81 (m, 1 H), 7.65 (dd, J = 7.7, 1.6 Hz, 1 H), 7.37 (t, J = 7.7 Hz, 1 H), 3.76 (s, 3 H), 3.18- 3.27 (m, 2 H), 3.01-3.18 (m, 3 H), 1.79-1.89 (m, 2 H), 1.54-1.68 (m, 2 H) |
| 33 | 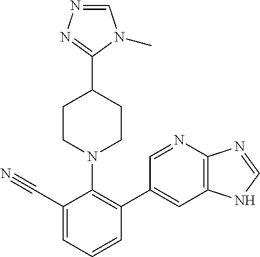 [SMILES: Cn1cnnc1C1CCN(c2c(C#N)cccc2-c2cnc3nc[nH]c3c2)CC1] | C₂₁H₂₀N₈ (M = 384.4 g/mol) ESI-MS: 385 [M + H]+ Rt (HPLC): 0.60 min (method D) | 9.11 (s, 1 H), 8.77 (s, 1 H), 8.45 (d, J = 1.8 Hz, 1 H), 8.13 (d, J = 1.9 Hz, 1 H), 7.80 (dd, J = 7.7, 1.6 Hz, 1 H), 7.59 (dd, J = 7.7, 1.6 Hz, 1 H), 7.31 (t, J = 7.7 Hz, 1 H), 3.72 (s, 3 H), 3.21- 3.29 (m, 2 H), 3.02 (tt, J = 11.7, 3.4 Hz, 1 H), 2.82- 2.96 (m, 2 H), 1.76-1.85 (m, 2 H), 1.57-1.72 (m, 2 H) |
| 35 | 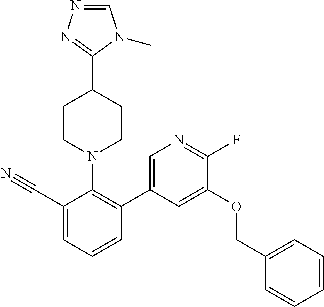 [SMILES: Cn1cnnc1C1CCN(c2c(C#N)cccc2-c2cnc(F)c(OCc3ccccc3)c2)CC1] | C₂₇H₂₅FN₆O (M = 468.5 g/mol) ESI-MS: 469 [M + H]+ Rt (HPLC): 0.55 min (method C) | 8.32 (s, 1 H), 7.86 (br d, J = 9.6 Hz, 1 H), 7.74-7.80 (m, 2 H), 7.51-7.56 (m, 3 H), 7.35-7.45 (m, 3 H), 7.27 (t, J = 7.7 Hz, 1 H), 5.32 (s, 2 H), 3.59 (s, 3 H), 3.16-3.24 (m, 2 H), 2.76- 2.95 (m, 3 H), 1.64-1.79 (m, 4 H) |
| 36 | 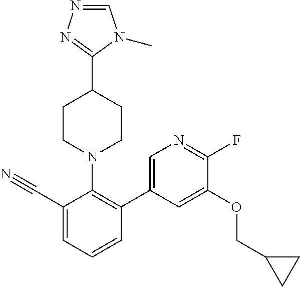 [SMILES: Cn1cnnc1C1CCN(c2c(C#N)cccc2-c2cnc(F)c(OCC3CC3)c2)CC1] | C₂₄H₂₅FN₆O (M = 432.5 g/mol) ESI-MS: 433 [M + H]+ Rt (HPLC): 0.78 min (method O) | 8.29 (s, 1 H), 7.77 (dd, J = 7.7, 1.6 Hz, 1 H), 7.69- 7.75 (m, 2 H), 7.54 (dd, J = 7.7, 1.6 Hz, 1 H), 7.27 (t, J = 7.7 Hz, 1 H), 4.04 (d, J = 7.2 Hz, 2 H), 3.58 (s, 3 H), 3.19-3.27 (m, 2 H), 2.99 (br t, J = 11.0 Hz, 2 H), 2.87 (tt, J = 11.3, 3.7 Hz, 1 H), 1.60-1.82 (m, 4 H), 1.21-1.33 (m, 1 H), 0.56- 0.63 (m, 2 H), 0.34-0.41 (m, 2 H) |
| 39 | 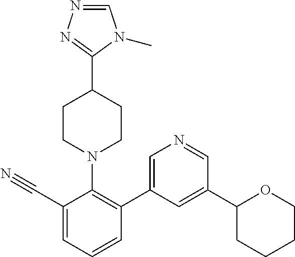 [SMILES: Cn1cnnc1C1CCN(c2c(C#N)cccc2-c2cncc(C3CCCCO3)c2)CC1] | C₂₅H₂₈N₆O (M = 428.5 g/mol) ESI-MS: 429 [M + H]+ Rt (HPLC): 0.66 min (method D) | 9.15 (s, 1 H), 8.64 (br s, 1 H), 8.61 (br s, 1 H), 8.00 (br s, 1 H), 7.80 (dd, J = 7.7, 1.5 Hz, 1 H), 7.60 (dd, J = 7.7, 1.6 Hz, 1 H), 7.33 (t, J = 7.7 Hz, 1 H), 4.51 (dd, J = 11.0, 1.6 Hz, 1 H), 3.99- 4.08 (m, 1 H), 3.74 (s, 3 H), 3.51-3.61 (m, 1 H), 3.20 (br d, J = 11.8 Hz, 2 H), 2.97- 3.10 (m, 3 H), 1.77-1.98 (m, 4 H), 1.45-1.74 (m, 6 H) |
| 40 | 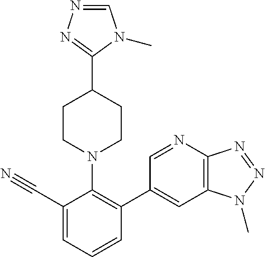 [SMILES: Cn1cnnc1C1CCN(c2c(C#N)cccc2-c2cnc3nnn(C)c3c2)CC1] | C₂₁H₂₁N₉ (M = 399.5 g/mol) ESI-MS: 400 [M + H]+ Rt (HPLC): 0.49 min (method M) | 9.11 (s, 1 H), 8.75 (d, J = 1.9 Hz, 1 H), 8.48 (d, J = 1.9 Hz, 1 H), 7.85 (dd, J = 7.7, 1.6 Hz, 1 H), 7.67 (dd, J = 7.6, 1.6 Hz, 1 H), 7.37 (t, J = 7.7 Hz, 1 H), 4.39 (s, 3 H), 3.71 (s, 3 H), 3.24 (br d, J = 12.5 Hz, 2 H), 3.12- 2.93 (m, 3 H), 1.79 (br d, J = 10.4 Hz, 2 H), 1.64-1.49 (m, 2 H) |
| 42 | 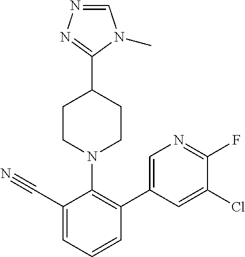 [SMILES: Cn1cnnc1C1CCN(c2c(C#N)cccc2-c2cnc(F)c(Cl)c2)CC1] | C₂₀H₁₈ClFN₆ (M = 396.9 g/mol) ESI-MS: 397 [M + H]+ Rt (HPLC): 0.58 min (method E) | 9.04 (s, 1 H), 8.33 (dd, J = 9.0, 2.2 Hz, 1 H), 8.23- 8.27 (m, 1 H), 7.82 (dd, J = 7.7, 1.5 Hz, 1 H), 7.62 (dd, J = 7.6, 1.6 Hz, 1 H), 7.33 (t, J = 7.7 Hz, 1 H), 3.74 (s, 3 H), 3.18-3.28 (m, 2 H), 2.97-3.12 (m, 3 H), 1.81-1.90 (m, 2 H), 1.53-1.68 (m, 2 H) |
| 43 | 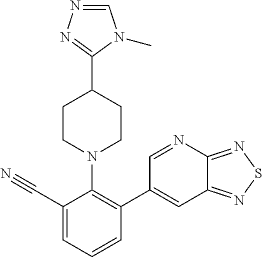 [SMILES: Cn1cnnc1C1CCN(c2c(C#N)cccc2-c2cnc3nsnc3c2)CC1] | C₂₀H₁₈N₈S (M = 402.5 g/mol) ESI-MS: 403 [M + H]+ Rt (HPLC): 0.69 min (method D) | 9.19 (d, J = 2.2 Hz, 1 H), 9.00 (s, 1 H), 8.59 (d, J = 2.2 Hz, 1 H), 7.89 (dd, J = 7.7, 1.6 Hz, 1 H), 7.77 (dd, J = 7.7, 1.6 Hz, 1 H), 7.41 (t, J = 7.7 Hz, 1 H), 3.69 (s, 3 H), 3.27-3.37 (m, 2 H), 3.10-3.20 (m, 2 H), 3.05 (tt, J = 11.8, 3.5 Hz, 1 H), 1.74-1.85 (m, 2 H), 1.45- 1.61 (m, 2 H) |
| 44 | 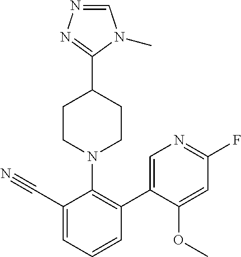 [SMILES: COc1cc(F)ncc1-c1cccc(C#N)c1N1CCC(c2nncn2C)CC1] | C₂₁H₂₁FN₆O (M = 392.4 g/mol) ESI-MS: 393 [M + H]+ Rt (HPLC): 0.79 min (method D) | 9.05 (s, 1 H), 7.94 (s, 1 H), 7.77 (dd, J = 7.7, 1.6 Hz, 1 H), 7.43 (dd, J = 7.7, 1.6 Hz, 1 H), 7.26 (t, J = 7.6 Hz, 1 H), 6.99 (s, 1 H), 3.89 (s, 3 H), 3.73 (s, 3 H), 3.12- 3.21 (m, 2 H), 3.04 (tt, J = 11.6, 3.4 Hz, 1 H), 2.91 (br t, J = 11.0 Hz, 2 H), 1.79- 1.89 (m, 2 H), 1.52-1.67 (m, 2 H) |
| 45 | 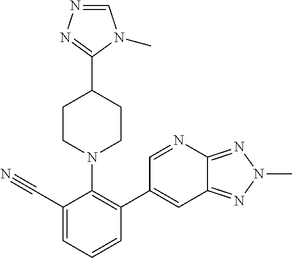 [SMILES: Cn1nc2cc(-c3cccc(C#N)c3N3CCC(c4nncn4C)CC3)cnc2n1] | C₂₁H₂₁N₉ (M = 399.5 g/mol) ESI-MS: 400 [M + H]+ Rt (HPLC): 3.45 min (method N) | 8.80 (d, J = 2.0 Hz, 1 H), 8.44 (d, J = 2.2 Hz, 1 H), 8.26 (s, 1 H), 7.82 (dd, J = 7.7, 1.6 Hz, 1 H), 7.66 (dd, J = 7.6, 1.6 Hz, 1 H), 7.34 (t, J = 7.7 Hz, 1 H), 4.57 (s, 3 H), 3.54 (s, 3 H), 3.25 (br d, J = 12.3 Hz, 2 H), 3.01 (br t, J = 10.4 Hz, 2 H), 2.80 (tt, J = 11.5, 3.6 Hz, 1 H), 1.77-1.65 (m, 2 H), 1.64-1.49 (m, 2 H) |
| 46 | 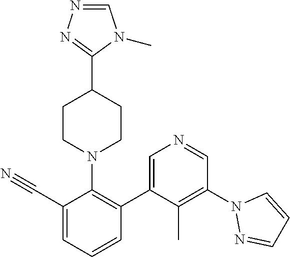 [SMILES: Cc1c(-c2cccc(C#N)c2N2CCC(c3nncn3C)CC2)cncc1-n1cccn1] | C₂₄H₂₄N₈ (M = 424.5 g/mol) ESI-MS: 425 [M + H]+ Rt (HPLC): 0.59 min (method J) | 9.04 (s, 1 H), 8.60 (s, 1 H), 8.46 (s, 1 H), 8.17 (d, J = 2.2 Hz, 1 H), 7.84 (dd, J = 7.7, 1.6 Hz, 1 H), 7.81 (d, J = 1.5 Hz, 1 H), 7.55 (dd, J = 7.6, 1.6 Hz, 1 H), 7.34 (t, J = 7.7 Hz, 1 H), 6.56-6.62 (m, 1 H), 3.72 (s, 3 H), 3.33 (br d, J = 12.0 Hz, 1 H), 3.09- 3.19 (m, 2 H), 3.05 (tt, J = 11.7, 3.5 Hz, 1 H), 2.85 (br t, J = 11.3 Hz, 1 H), 2.01 (s, 3 H), 1.87 (br d, J = 12.8 Hz, 1 H), 1.76 (br d, J = 11.8 Hz, 1 H), 1.52-1.65 (m, 1 H), 1.37-1.50 (m, 1 H) |
| 47 | 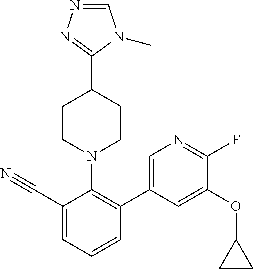 [SMILES: Cn1cnnc1C1CCN(c2c(C#N)cccc2-c2cnc(F)c(OC3CC3)c2)CC1] | C₂₃H₂₃FN₆O (M = 418.5 g/mol) ESI-MS: 419 [M+H]+ Rt (HPLC): 0.72 min (method I) | 8.30 (s, 1 H), 7.92 (dd, J = 9.9, 1.7 Hz, 1 H), 7.76- 7.82 (m, 2 H), 7.56 (dd, J = 7.6, 1.6 Hz, 1 H), 7.28 (t, J = 7.7 Hz, 1 H), 4.12 (quin, J = 4.4 Hz, 1 H), 3.58 (s, 3 H), 3.20-3.28 (m, 2 H), 2.95-3.07 (m, 2 H), 2.87 (tt, J = 11.1, 3.8 Hz, 1 H), 1.63-1.85 (m, 4 H), 0.84 (d, J = 4.4 Hz, 4 H) |
| 48 | 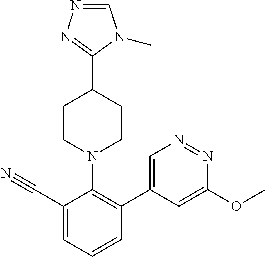 [SMILES: COc1cc(-c2cccc(C#N)c2N2CCC(c3nncn3C)CC2)cnn1] | C₂₀H₂₁N₇O (M = 375.4 g/mol) ESI-MS: 376 [M + H]+ Rt (HPLC): 0.65 min (method D) | 9.13 (s, 1 H), 8.97 (d, J = 1.6 Hz, 1 H), 7.86 (dd, J = 7.7, 1.6 Hz, 1 H), 7.63 (dd, J = 7.7, 1.6 Hz, 1 H), 7.34 (t, J = 7.7 Hz, 1 H), 7.29 (d, J = 1.6 Hz, 1 H), 4.09 (s, 3 H), 3.76 (s, 3 H), 3.21- 3.33 (m, 2 H), 3.00-3.19 (m, 3 H), 1.81-1.93 (m, 2 H), 1.54-1.74 (m, 2 H) |
| 49 | 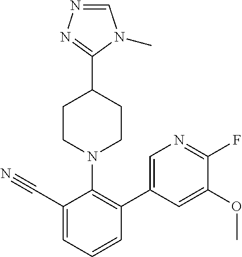 [SMILES: COc1cc(-c2cccc(C#N)c2N2CCC(c3nncn3C)CC2)cnc1F] | C₂₁H₂₁FN₆O (M = 392.4 g/mol) ESI-MS: 393 [M + H]+ Rt (HPLC): 0.68 min (method O) | 8.30 (s, 1 H), 7.72 - 7.80 (m, 3 H), 7.57 (dd, J = 7.7, 1.5 Hz, 1 H), 7.28 (t, J = 7.7 Hz, 1 H), 3.97 (s, 3 H), 3.58 (s, 3 H), 3.20-3.28 (m, 2 H), 3.00 (br t, J = 10.7 Hz, 2 H), 2.89 (tt, J = 11.3, 3.9 Hz, 1 H), 1.63-1.82 (m, 4 H) |
| 50 | 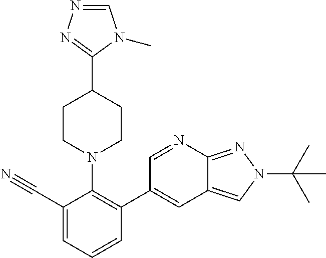 [SMILES: Cn1cnnc1C1CCN(c2c(C#N)cccc2-c2cnc3nn(C(C)(C)C)cc3c2)CC1] | C₂₅H₂₈N₈ (M = 440.6 g/mol) ESI-MS: 441 [M + H]+ Rt (HPLC): 0.46 min (method C) | 8.59 (d, J = 2.2 Hz, 1 H), 8.58 (s, 1 H), 8.27 (s, 1 H), 8.14 (d, J = 2.3 Hz, 1 H), 7.77 (dd, J = 7.7, 1.6 Hz, 1 H), 7.55 (dd, J = 7.6, 1.6 Hz, 1 H), 7.29 (t, J = 7.7 Hz, 1 H), 3.55 (s, 3 H), 3.22- 3.26 (m, 2 H), 2.90-3.02 (m, 2 H), 2.74-2.89 (m, 1 H), 1.72-1.77 (m, 4 H), 1.71 (s, 9 H) |
| 51 | 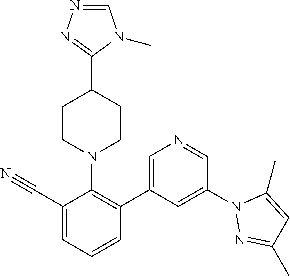 [SMILES: Cc1cc(C)n(-c2cncc(-c3cccc(C#N)c3N3CCC(c4nncn4C)CC3)c2)n1] | C₂₅H₂₆N₈ (M = 438.5 g/mol) ESI-MS: 439 [M + H]+ Rt (HPLC): 0.45 min (method C) | 8.77 (d, J = 2.4 Hz, 1 H), 8.59 (d, J = 1.9 Hz, 1 H), 8.28 (s, 1 H), 7.99 (t, J = 2.2 Hz, 1 H), 7.81 (dd, J = 7.7, 1.5 Hz, 1 H), 7.60 (dd, J = 7.7, 1.6 Hz, 1 H), 7.31 (t, J = 7.7 Hz, 1 H), 6.14 (s, 1 H), 3.57 (s, 3 H), 3.21- 3.28 (m, 2 H), 3.00 (br t, J = 11.1 Hz, 2 H), 2.83 (tt, J = 11.3, 3.6 Hz, 1 H), 2.39 (s, 3 H), 2.19 (s, 3 H), 1.73- 1.81 (m, 2 H), 1.60-1.73 (m, 2 H) |
| 52 | 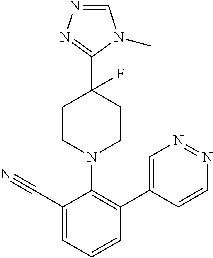 [SMILES: Cn1cnnc1C1(F)CCN(c2c(C#N)cccc2-c2ccnnc2)CC1] | C₁₉H₁₈FN₇ (M = 363.4 g/mol) ESI-MS: 364 [M + H]+ Rt (HPLC): 0.37 min (method C) | 9.38 (dd, J = 2.2, 1.2 Hz, 1 H), 9.35 (dd, J = 5.3, 1.2 Hz, 1 H), 8.59 (s, 1 H), 7.89 (dd, J = 7.7, 1.6 Hz, 1 H), 7.82 (dd, J = 5.3, 2.3 Hz, 1 H), 7.67 (dd, J = 7.7, 1.6 Hz, 1 H), 7.40 (t, J = 7.7 Hz, 1 H), 3.74 (d, J = 1.6 Hz, 3 H), 3.20-3.32 (m, 2 H), 3.08- 3.17 (m, 2 H), 1.99-2.21 (m, 4 H) |
| 53 | 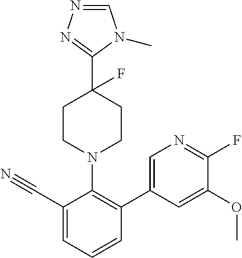 [SMILES: COc1cc(-c2cccc(C#N)c2N2CCC(F)(c3nncn3C)CC2)cnc1F] | C₂₁H₂₀F₂N₆O (M = 410.4 g/mol) ESI-MS: 411 [M + H]+ Rt (HPLC): 0.71 min (method H) | 8.57 (s, 1 H), 7.82 (dd, J = 7.7, 1.6 Hz, 1 H), 7.80- 7.75 (m, 2 H), 7.60 (dd, J = 1.6, 7.6 Hz, 1 H), 7.32 (t, J = 7.7 Hz, 1 H), 3.92 (s, 3 H), 3.73 (d, J = 1.5 Hz, 3 H), 3.26-3.10 (m, 4 H), 2.26-2.09 (m, 4 H) |
| 54 | 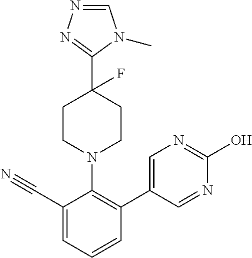 [SMILES: Cn1cnnc1C1(F)CCN(c2c(C#N)cccc2-c2cnc(O)nc2)CC1] | C₁₉H₁₈FN₇O (M = 379.4 g/mol) ESI-MS: 380 [M + H]+ Rt (HPLC): 0.35 min (method C) | 8.56 (s, 1 H), 8.44 (s, 2 H), 7.77 (dd, J = 7.7, 1.6 Hz, 1 H), 7.63 (dd, J = 7.7, 1.6 Hz, 1 H), 7.34 (t, J = 7.7 Hz, 1 H), 3.77 (d, J = 1.5 Hz, 3 H), 3.32-3.47 (m, 2 H), 3.14- 3.24 (m, 2 H), 2.07-2.30 (m, 4 H) |
| 56 | 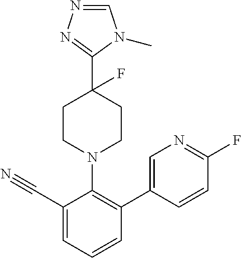 [SMILES: Cn1cnnc1C1(F)CCN(c2c(C#N)cccc2-c2ccc(F)nc2)CC1] | C₂₀H₁₈F₂N₆ (M = 380.4 g/mol) ESI-MS: 381 [M + H]+ Rt (HPLC): 0.80 min (method G) | 8.47 (s, 1 H), 8.30 (d, J = 2.3 Hz, 1 H), 8.08 (td, J = 8.2, 2.5 Hz, 1 H), 7.82 (dd, J = 7.7, 1.6 Hz, 1 H), 7.58 (dd, J = 7.6, 1.5 Hz, 1 H), 7.28-7.37 (m, 2 H), 3.72 (d, J = 1.4 Hz, 3 H), 3.14- 3.25 (m, 2 H), 3.06-3.14 (m, 2 H), 2.03-2.21 (m, 4 H) |
| 57 | 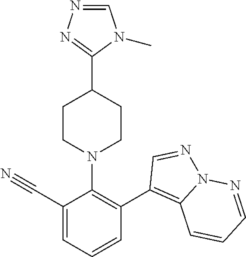 [SMILES: Cn1cnnc1C1CCN(c2c(C#N)cccc2-c2cnn3ncccc23)CC1] | C₂₁H₂₀N₈ (M = 384.4 g/mol) ESI-MS: 385 [M + H]+ Rt (HPLC): 0.52 min (method P) | 8.50 (dd, J = 4.4, 1.8 Hz, 1 H), 8.27 (s, 1 H), 8.25 (s, 1 H), 8.18 (dd, J = 9.0, 1.9 Hz, 1 H), 7.74 (dd, J = 7.7, 1.6 Hz, 1 H), 7.61 (dd, J = 7.7, 1.6 Hz, 1 H), 7.28 (t, J = 7.7 Hz, 1 H), 7.24 (dd, J = 9.0, 4.4 Hz, 1 H), 3.54 (s, 3 H), 3.19-3.26 (m, 2 H), 2.97- 3.08 (m, 2 H), 2.79 (tt, J = 11.5, 3.5 Hz, 1 H), 1.67- 1.74 (m, 2 H), 1.50-1.62 (m, 2 H) |
| 59 | 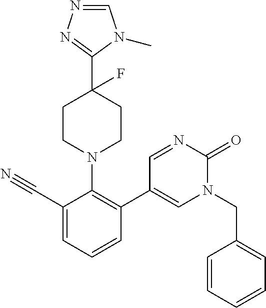 [SMILES: Cn1cnnc1C1(F)CCN(c2c(C#N)cccc2-c2cnc(=O)n(Cc3ccccc3)c2)CC1] | C₂₆H₂₄FN₇O (M = 469.5 g/mol) ESI-MS: 470 [M + H]+ Rt (HPLC): 0.46 min (method C) | 8.75 (d, J = 3.2 Hz, 1 H), 8.56 (s, 1 H), 8.51 (d, J = 3.3 Hz, 1 H), 7.79 (dd, J = 7.7, 1.5 Hz, 1 H), 7.62 (dd, J = 7.7, 1.6 Hz, 1 H), 7.26- 7.40 (m, 6 H), 5.10 (s, 2 H), 3.76 (d, J = 1.4 Hz, 3 H), 3.30-3.43 (m, 2 H), 3.13- 3.23 (m, 2 H), 2.07-2.27 (m, 4 H) |
| 60 | 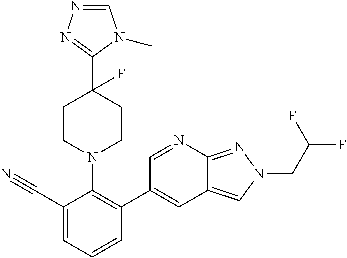 [SMILES: Cn1cnnc1C1(F)CCN(c2c(C#N)cccc2-c2cnc3nn(CC(F)F)cc3c2)CC1] | C₂₃H₂₁F₃N₈ (M = 466.5 g/mol) ESI-MS: 467 [M + H]+ Rt (HPLC): 0.60 min (method L) | 8.68 (d, J = 2.2 Hz, 1 H), 8.58 (s, 1 H), 8.44 (s, 1 H), 8.25 (d, J = 2.3 Hz, 1 H), 7.81 (dd, J = 7.7, 1.6 Hz, 1 H), 7.62 (dd, J = 7.7, 1.6 Hz, 1 H), 7.34 (t, J = 7.7 Hz, 1 H), 6.61 (tt, J = 54.8, 3.7 Hz, 1 H), 5.04 (td, J = 14.8, 3.9 Hz, 2 H), 3.68 (d, J = 1.5 Hz, 3 H), 3.11-3.21 (m, 4 H), 2.05-2.18 (m, 4 H) |
| 61 | 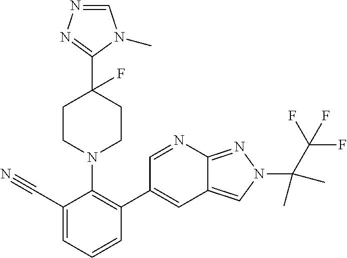 [SMILES: Cn1cnnc1C1(F)CCN(c2c(C#N)cccc2-c2cnc3nn(C(C)(C)C(F)(F)F)cc3c2)CC1] | C₂₅H₂₄F₄N₈ (M = 512.5 g/mol) ESI-MS: 513 [M + H]+ Rt (HPLC): 0.75 min (method J) | 8.89 (s, 1 H), 8.72 (d, J = 2.3 Hz, 1 H), 8.45 (s, 1 H), 8.21 (d, J = 2.2 Hz, 1 H), 7.82 (dd, J = 7.7, 1.5 Hz, 1 H), 7.63 (dd, J = 7.7, 1.6 Hz, 1 H), 7.35 (t, J = 7.7 Hz, 1 H), 3.69 (d, J = 1.5 Hz, 3 H), 3.13-3.23 (m, 4 H), 2.06- 2.20 (m, 4 H), 2.03 (s, 6 H) |
| 62 | 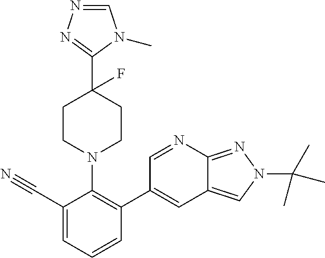 [SMILES: Cn1cnnc1C1(F)CCN(c2c(C#N)cccc2-c2cnc3nn(C(C)(C)C)cc3c2)CC1] | C₂₅H₂₇FN₈ (M = 458.5 g/mol) ESI-MS: 459 [M + H]+ Rt (HPLC): 0.72 min (method P) | 8.67 (d, J = 2.2 Hz, 1 H), 8.63 (s, 1 H), 8.55 (s, 1 H), 8.22 (d, J = 2.2 Hz, 1 H), 7.81 (dd, J = 7.7, 1.6 Hz, 1 H), 7.60 (dd, J = 7.7, 1.6 Hz, 1 H), 7.34 (t, J = 7.7 Hz, 1 H), 3.70 (d, J = 1.5 Hz, 3 H), 3.12- 3.24 (m, 4 H), 2.07-2.23 (m, 4 H), 1.72 (s, 9 H) |
| 63 | 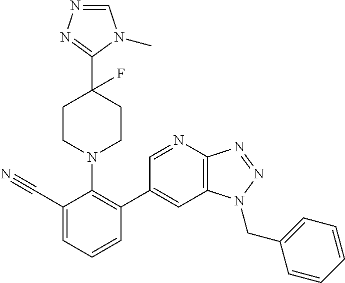 [SMILES: Cn1cnnc1C1(F)CCN(c2c(C#N)cccc2-c2cnc3nnn(Cc4ccccc4)c3c2)CC1] | C₂₇H₂₄FN₉ (M = 493.6 g/mol) ESI-MS: 494 [M + H]+ Rt (HPLC): 0.58 min (method C) | 8.79 (d, J=1.8 Hz, 1 H), 8.52 (s, 1 H), 8.47 (d, J = 1.8 Hz, 1 H), 7.88 (dd, J = 7.7, 1.5 Hz, 1 H), 7.64 (dd, J = 7.7, 1.5 Hz, 1 H), 7.28- 7.44 (m, 6 H), 6.02 (s, 2 H), 3.69 (d, J = 1.3 Hz, 3 H), 3.03-3.18 (m, 4 H), 1.91- 2.13 (m, 4 H) |
| 64 | 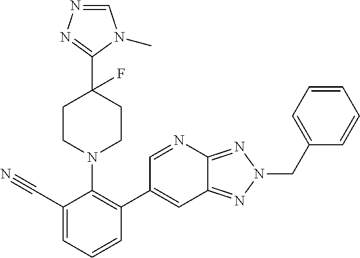 [SMILES: Cn1cnnc1C1(F)CCN(c2c(C#N)cccc2-c2cnc3nn(Cc4ccccc4)nc3c2)CC1] | C₂₇H₂₄FN₉ (M = 493.6 g/mol) ESI-MS: 494 [M + H]+ Rt (HPLC): 0.58 min (method C) | 8.85 (d, J = 2.0 Hz, 1 H), 8.52 (s, 1 H), 8.48 (d, J = 2.0 Hz, 1 H), 7.85 (dd, J = 7.7, 1.6 Hz, 1 H), 7.68 (dd, J = 7.7, 1.5 Hz, 1 H), 7.45- 7.50 (m, 2 H), 7.33-7.44 (m, 4 H), 6.04 (s, 2 H), 3.69 (d, J = 1.4 Hz, 3 H), 3.10- 3.27 (m, 4 H), 1.93-2.17 (m, 4 H) |
| 66 | 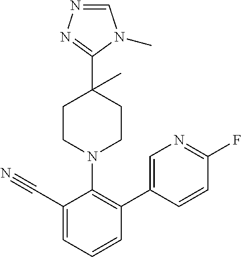 [SMILES: Cn1cnnc1C1(C)CCN(c2c(C#N)cccc2-c2ccc(F)nc2)CC1] | C₂₁H₂₁FN₆ (M = 376.4 g/mol) ESI-MS: 377 [M + H]+ Rt (HPLC): 0.47 min (method C) | 8.89 (s, 1 H), 8.26 (d, J = 2.3 Hz, 1 H), 8.03 (td, J = 8.2, 2.4 Hz, 1 H), 7.78 (dd, J = 7.7, 1.6 Hz, 1 H), 7.56 (dd, J = 7.7, 1.6 Hz, 1 H), 7.31 (t, J = 7.7 Hz, 1 H), 7.27 (dd, J = 8.4, 2.7 Hz, 1 H), 3.79 (s, 3 H), 2.93- 3.14 (m, 4 H), 2.06-2.16 (m, 2 H), 1.60-1.74 (m, 2 H), 1.31 (s, 3 H) |
| 67 | 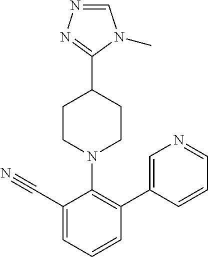 [SMILES: Cn1cnnc1C1CCN(c2c(C#N)cccc2-c2cccnc2)CC1] | C₂₀H₂₀N₆ (M = 344.4 g/mol) ESI-MS: 345 [M + H]+ Rt (HPLC): 0.30 min (method C) | 8.58-8.63 (m, 2 H), 8.29 (s, 1 H), 7.84 (dt, J = 7.8, 1.9 Hz, 1 H), 7.78 (dd, J = 7.7, 1.6 Hz, 1 H), 7.48-7.55 (m, 2 H), 7.29 (t, J = 7.7 Hz, 1 H), 3.57 (s, 3 H), 3.15- 3.22 (m, 2 H), 2.89-3.00 (m, 2 H), 2.82 (tt, J = 11.3, 4.0 Hz, 1 H), 1.59-1.78 (m, 4 H) |
| 68 | 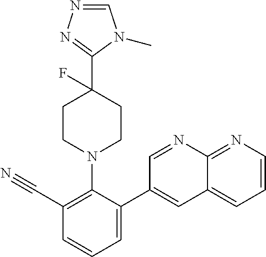 [SMILES: Cn1cnnc1C1(F)CCN(c2c(C#N)cccc2-c2cnc3ncccc3c2)CC1] | C₂₃H₂₀FN₇ (M = 413.5 g/mol) ESI-MS: 414 [M + H]+ Rt (HPLC): 0.69 min (method D) | 9.23 (d, J = 2.4 Hz, 1 H), 9.17 (dd, J = 4.4, 1.9 Hz, 1 H), 8.64 (dd, J = 8.2, 1.9 Hz, 1 H), 8.61 (d, J = 2.4 Hz, 1 H), 8.56 (s, 1 H), 7.89 (dd, J = 7.7, 1.6 Hz, 1 H), 7.73- 7.81 (m, 2 H), 7.42 (t, J = 7.7 Hz, 1 H), 3.69 (d, J = 1.6 Hz, 3 H), 3.21-3.35 (m, 2 H), 3.19 (br s, 2 H), 1.94-2.17 (m, 4 H) |
| 69 | 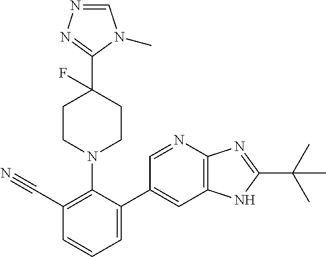 | C₂₅H₂₇FN₈ (M = 413.5 g/mol) ESI-MS: 381 [M + H]+ Rt (HPLC): 0.74 min (method D) | 8.50 (s, 1 H), 8.48 (d, J = 1.1 Hz, 1 H), 8.08 (d, J = 1.6 Hz, 1 H), 7.83 (dd, J = 7.7, 1.6 Hz, 1 H), 7.59 (dd, J = 7.7, 1.6 Hz, 1 H), 7.35 (t, J = 7.7 Hz, 1 H), 3.70 (d, J = 1.5 Hz, 3 H), 3.05-3.21 (m, 4 H), 2.00-2.25 (m, 4 H), 1.49 (s, 9 H) |
| 70 | 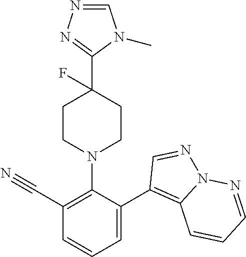 [SMILES: Cn1cnnc1C1(F)CCN(c2c(C#N)cccc2-c2cnn3ncccc23)CC1] | C₂₁H₁₉FN₈ (M = 402.4 g/mol) ESI-MS: 403 [M + H]+ Rt (HPLC): 0.83 min (method E) | 8.50 (dd, J = 4.3, 1.6 Hz, 1 H), 8.44 (s, 1 H), 8.27 (s, 1 H), 8.21 (dd, J = 9.0, 1.6 Hz, 1 H), 7.77 (dd, J = 7.7, 1.2 Hz, 1 H), 7.63 (dd, J = 7.6, 1.1 Hz, 1 H), 7.32 (t, J = 7.7 Hz, 1 H), 7.23 (dd, J = 9.1, 4.5 Hz, 1 H), 3.69 (d, J = 0.9 Hz, 3 H), 3.18-3.26 (m, 2 H), 3.09-3.18 (m, 2 H), 1.95- 2.15 (m, 4 H) |
| 71 | 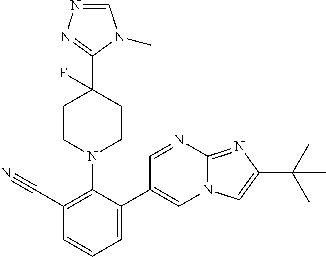 [SMILES: Cn1cnnc1C1(F)CCN(c2c(C#N)cccc2-c2cnc3nc(C(C)(C)C)cn3c2)CC1] | C₂₅H₂₇FN₈ (M = 458.5 g/mol) ESI-MS: 459 [M + H]+ Rt (HPLC): 0.69 min (method E) | 8.95 (d, J = 2.4 Hz, 1 H), 8.57 (d, J = 2.3 Hz, 1 H), 8.46 (s, 1 H), 7.84 (dd, J = 7.7, 1.6 Hz, 1 H), 7.64- 7.70 (m, 2 H), 7.37 (t, J = 7.7 Hz, 1 H), 3.71 (d, J = 1.5 Hz, 3 H), 3.26-3.29 (m, 2 H), 3.17-3.25 (m, 2 H), 2.09-2.24 (m, 4 H), 1.35 (s, 9 H) |
| 72 | 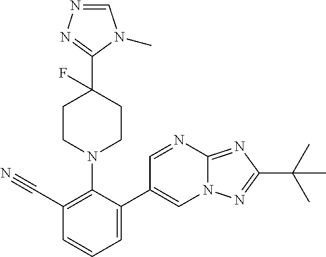 [SMILES: Cn1cnnc1C1(F)CCN(c2c(C#N)cccc2-c2cnc3nc(C(C)(C)C)nn3c2)CC1] | C₂₄H₂₆FN₉ (M = 459.5 g/mol) ESI-MS: 460 [M + H]+ Rt (HPLC): 0.71 min (method Q) | 9.44 (d, J = 2.3 Hz, 1H), 8.96 (d, J = 2.2 Hz, 1H), 8.53 (s, 1H), 7.88 (dd, J = 1.5, 7.7 Hz, 1H), 7.76 (dd, J = 1.5, 7.6 Hz, 1H), 7.41 (t, J = 7.7 Hz, 1H), 3.72 (d, J = 1.4 Hz, 3H), 3.42 - 3.28 (m, 2H), 3.25- 3.18 (m, 2H), 2.23- 1.98 (br d, J = 11.4 Hz, 4H), 1.44 (s, 9H) |
| 73 | 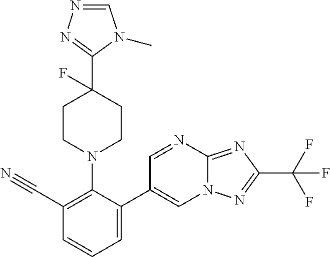 [SMILES: Cn1cnnc1C1(F)CCN(c2c(C#N)cccc2-c2cnc3nc(C(F)(F)F)nn3c2)CC1] | C₂₁H₁₇F₄N₉ (M = 471.4 g/mol) ESI-MS: 472 [M + H]+ Rt (HPLC): 0.67 min (method O) | 9.69 (d, J = 2.3 Hz, 1H), 9.25 (d, J = 2.3 Hz, 1H), 8.46 (s, 1H), 7.92 (dd, J = 1.6, 7.8 Hz, 1H), 7.78 (dd, J = 1.6, 7.7 Hz, 1H), 7.46 (t, J = 7.7 Hz, 1H), 3.71 (d, J = 1.4 Hz, 3H), 3.47-3.34 (m, 2H), 3.27- 3.17 (m, 2H), 2.20- 1.98 (m, 4H) |
| 74 | 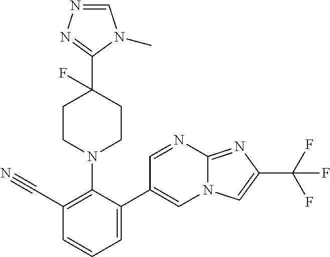 [SMILES: Cn1cnnc1C1(F)CCN(c2c(C#N)cccc2-c2cnc3nc(C(F)(F)F)cn3c2)CC1] | C₂₂H₁₈F₄N₈ (M = 470.4 g/mol) ESI-MS: 471 [M + H]+ Rt (HPLC): 0.48 min (method C) | 9.09 (d, J = 2.3 Hz, 1 H), 8.88 (d, J = 2.3 Hz, 1 H), 8.57 (s, 1 H), 8.53 (s, 1 H), 7.89 (dd, J = 7.7, 1.4 Hz, 1 H), 7.73 (dd, J = 7.7, 1.3 Hz, 1 H), 7.42 (t, J = 7.7 Hz, 1 H), 3.72 (d, J = 0.9 Hz, 3 H), 3.31-3.44 (m, 2 H), 3.22 (br d, J = 12.3 Hz, 2 H), 2.02- 2.23 (m, 4 H) |
| 75 | 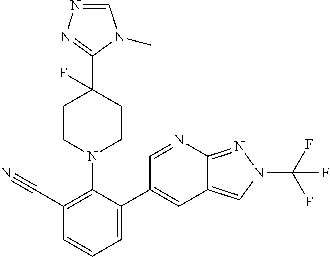 [SMILES: Cn1cnnc1C1(F)CCN(c2c(C#N)cccc2-c2cnc3nn(C(F)(F)F)cc3c2)CC1] | C₂₂H₁₈F₄N₈ (M = 470.4 g/mol) ESI-MS: 471 [M + H]+ Rt (HPLC): 0.69 min (method J) | 9.29 (s, 1 H), 8.91 (d, J = 2.2 Hz, 1 H), 8.45 (s, 1 H), 8.31 (d, J = 2.2 Hz, 1 H), 7.85 (dd, J = 7.7, 1.6 Hz, 1 H), 7.68 (dd, J = 7.7, 1.6 Hz, 1 H), 7.38 (t, J = 7.7 Hz, 1 H), 3.69 (d, J = 1.4 Hz, 3 H), 3.24-3.29 (m, 2 H), 3.15- 3.23 (m, 2 H), 1.96-2.19 (m, 4 H) |
| 76 | 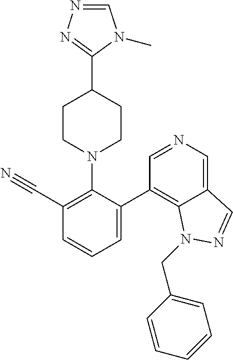 [SMILES: Cn1cnnc1C1CCN(c2c(C#N)cccc2-c2cncc3cnn(Cc4ccccc4)c23)CC1] | C₂₈H₂₆N₈ (M = 474.6 g/mol) ESI-MS: 475 [M + H]+ Rt (HPLC): 0.92 min (method D) | 9.17 (s, 1 H), 8.51 (s, 1 H), 8.23 (s, 1 H), 8.14 (s, 1 H), 7.88 (dd, J = 7.7, 1.6 Hz, 1 H), 7.51 (dd, J = 7.5, 1.6 Hz, 1 H), 7.24 (t, J = 7.7 Hz, 1 H), 7.06-7.18 (m, 3 H), 6.42-6.49 (m, 2 H), 5.30 (d, J = 16.6 Hz, 1 H), 5.21 (d, J = 16.1 Hz, 1 H), 3.47 (s, 3 H), 3.25-3.29 (m, 1 H), 2.95 (br t, J = 11.8 Hz, 1 H), 2.62 (tt, J = 11.4, 3.4 Hz, 1 H), 2.30-2.40 (m, 2 H), 1.62 (br d, J = 11.5 Hz, 1 H), 1.40-1.54 (m, 1 H), 1.29 (br d, J = 12.3 Hz, 1 H), 0.76- 0.94 (m, 1 H) |
| 77 | 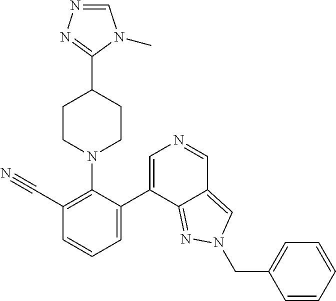 [SMILES: Cn1cnnc1C1CCN(c2c(C#N)cccc2-c2cncc3cn(Cc4ccccc4)nc23)CC1] | C₂₈H₂₆N₈ (M = 474.6 g/mol) ESI-MS: 475 [M + H]+ Rt (HPLC): 0.65 min (method L) | 9.21 (s, 1 H), 8.91 (s, 1 H), 8.26 (s, 1 H), 8.10 (s, 1 H), 7.76 (dd, J = 7.6, 1.6 Hz, 1 H), 7.54 (dd, J = 7.6, 1.6 Hz, 1 H), 7.31-7.36 (m, 5 H), 7.22 (t, J = 7.7 Hz, 1 H), 5.69 (br s, 2 H), 3.48 (s, 3 H), 3.32-3.36 (m, 2 H), 3.04-3.12 (m, 2 H), 2.33- 2.37 (m, 1 H), 1.39-1.56 (m, 4 H) |
| 78 | 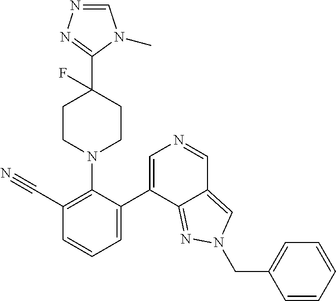 [SMILES: Cn1cnnc1C1(F)CCN(c2c(C#N)cccc2-c2cncc3cn(Cc4ccccc4)nc23)CC1] | C₂₈H₂₅FN₈ (M = 492.6 g/mol) ESI-MS: 493 [M + H]+ Rt (HPLC): 0.67 min (method O) | 9.21 (s, 1 H), 8.87 (s, 1 H), 8.42 (s, 1 H), 8.11 (s, 1 H), 7.80 (dd, J = 7.7, 1.6 Hz, 1 H), 7.58 (dd, J = 7.6, 1.6 Hz, 1 H), 7.26-7.33 (m, 6 H), 5.67 (s, 2 H), 3.63 (d, J = 1.5 Hz, 3 H), 2.93-3.03 (m, 2 H), 2.78-2.91 (m, 2 H), 1.73-1.93 (m, 4 H) |
| 79 | 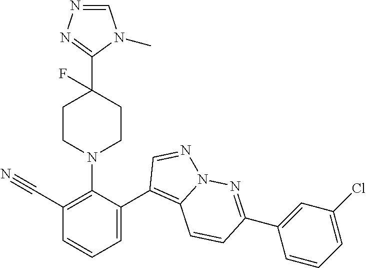 [SMILES: Cn1cnnc1C1(F)CCN(c2c(C#N)cccc2-c2cnn3nc(-c4cccc(Cl)c4)ccc23)CC1] | C₂₇H₂₂ClFN₈ (M = 513.0 g/mol) ESI-MS: 513 [M + H]+ Rt (HPLC): 0.88 min (method L) | 8.43 (s, 1 H), 8.31-8.37 (m, 2 H), 8.16-8.19 (m, 1 H), 8.08-8.13 (m, 1 H), 7.89 (d, J = 9.4 Hz, 1 H), 7.79 (dd, J = 7.7, 1.5 Hz, 1 H), 7.67 (dd, J = 7.6, 1.4 Hz, 1 H), 7.60-7.65 (m, 2 H), 7.34 (t, J = 7.7 Hz, 1 H), 3.68 (d, J = 1.1 Hz, 3 H), 3.21-3.28 (m, 2 H), 3.13- 3.21 (m, 2 H), 1.98-2.18 (m, 4 H) |
| 81 | 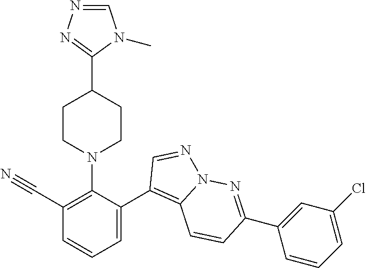 [SMILES: Cn1cnnc1C1CCN(c2c(C#N)cccc2-c2cnn3nc(-c4cccc(Cl)c4)ccc23)CC1] | C₂₇H₂₃ClN₈ (M = 495.0 g/mol) ESI-MS: 495 [M + H]+ Rt (HPLC): 0.77 min (method M) | 8.90-9.00 (m, 1 H), 8.30- 8.34 (m, 2 H), 8.18 (s, 1 H), 8.09-8.13 (m, 1 H), 7.91 (d, J = 9.5 Hz, 1 H), 7.77 (dd, J = 7.7, 1.5 Hz, 1 H), 7.66 (dd, J = 7.6, 1.5 Hz, 1 H), 7.60-7.64 (m, 2 H), 7.32 (t, J = 7.7 Hz, 1 H), 3.69 (s, 3 H), 3.24-3.32 (m, 2 H), 2.98-3.12 (m, 3 H), 1.75-1.86 (m, 2 H), 1.53-1.68 (m, 2 H) |
| 82 | 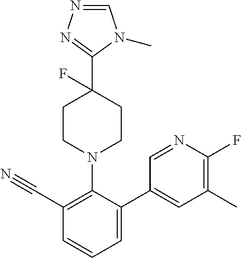 [SMILES: Cc1cc(-c2cccc(C#N)c2N2CCC(F)(c3nncn3C)CC2)cnc1F] | C₂₁H₂₀F₂N₆ (M = 394.4 g/mol) ESI-MS: 395 [M + H]+ Rt (HPLC): 0.72 min (method L) | 8.47 (s, 1 H), 8.07-8.11 (m, 1 H), 7.92-7.97 (m, 1 H), 7.80 (dd, J = 7.7, 1.6 Hz, 1 H), 7.57 (dd, J = 7.7, 1.6 Hz, 1 H), 7.32 (t, J = 7.7 Hz, 1 H), 3.72 (d, J = 1.6 Hz, 3 H), 3.14-3.24 (m, 2 H), 3.05-3.14 (m, 2 H), 2.29 (s, 3 H), 2.05-2.19 (m, 4 H) |
| 83 | 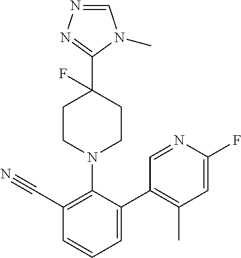 [SMILES: Cc1cc(F)ncc1-c1cccc(C#N)c1N1CCC(F)(c2nncn2C)CC1] | C₂₁H₂₀F₂N₆ (M = 394.4 g/mol) ESI-MS: 395 [M + H]+ Rt (HPLC): 0.70 min (method L) | 8.46 (s, 1 H), 8.04 (s, 1 H), 7.83 (dd, J = 7.7, 1.7 Hz, 1 H), 7.49 (dd, J = 7.6, 1.6 Hz, 1 H), 7.30-7.36 (m, 1 H), 7.22-7.25 (m, 1 H), 3.70 (d, J = 1.5 Hz, 3 H), 3.14- 3.27 (m, 2 H), 2.94-3.02 (m, 2 H), 2.34-2.43 (m, 2 H), 2.18 (s, 3 H), 2.00- 2.17 (m, 2 H) |
| 84 | 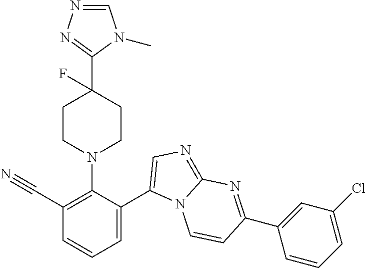 [SMILES: Cn1cnnc1C1(F)CCN(c2c(C#N)cccc2-c2cnc3nc(-c4cccc(Cl)c4)ccn23)CC1] | C₂₇H₂₂ClFN₈ (M = 513.0 g/mol) ESI-MS: 513 [M + H]+ Rt (HPLC): 0.76 min (method G) | 8.93 (d, J = 7.2 Hz, 1 H), 8.42 (s, 1 H), 8.33 (t, J = 1.7 Hz, 1 H), 8.26 (dt, J = 7.6, 1.5 Hz, 1 H), 8.21 (s, 1 H), 8.00 (d, J = 7.2 Hz, 1 H), 7.97 (dd, J = 7.9, 1.6 Hz, 1 H), 7.78 (dd, J = 7.6, 1.6 Hz, 1 H), 7.66-7.70 (m, 1 H), 7.61-7.66 (m, 1 H), 7.43 (t, J = 7.7 Hz, 1 H), 3.65 (d, J = 1.6 Hz, 3 H), 3.21-3.34 (m, 4 H), 1.99-2.14 (m, 2 H), 1.73-1.95 (m, 2 H) |
| 85 | 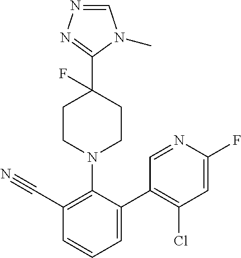 [SMILES: Cn1cnnc1C1(F)CCN(c2c(C#N)cccc2-c2cnc(F)cc2Cl)CC1] | C₂₀H₁₇ClF₂N₆ (M = 414.8 g/mol) ESI-MS: 415 [M + H]+ Rt (HPLC): 0.74 min (method J) | 8.46 (s, 1 H), 8.34 (s, 1 H), 7.88 (dd, J = 7.7, 1.6 Hz, 1 H), 7.72 (d, J = 2.3 Hz, 1 H), 7.56 (dd, J = 7.6, 1.6 Hz, 1 H), 7.37 (t, J = 7.7 Hz, 1 H), 3.71 (d, J = 1.6 Hz, 3 H), 3.01-3.22 (m, 4 H), 1.82- 2.22 (m, 4 H) |
| 86 | 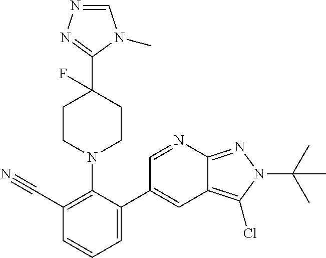 [SMILES: Cn1cnnc1C1(F)CCN(c2c(C#N)cccc2-c2cnc3nn(C(C)(C)C)c(Cl)c3c2)CC1] | C₂₅H₂₆ClFN₈ (M = 493.0 g/mol) ESI-MS: 493 [M + H]+ Rt (HPLC): 0.95 min (method G) | 8.73 (d, J = 2.0 Hz, 1 H), 8.45 (s, 1 H), 8.09 (d, J = 2.2 Hz, 1 H), 7.82 (dd, J = 7.7, 1.5 Hz, 1 H), 7.65 (dd, J = 7.7, 1.6 Hz, 1 H), 7.35 (t, J = 7.7 Hz, 1 H), 3.69 (d, J = 1.4 Hz, 3 H), 3.12-3.27 (m, 4 H), 2.02-2.20 (m, 4 H), 1.84 (s, 9 H) |

### Analytical HPLC Methods

### Method A

| | Vol % | Vol % | Flow |
|---|---|---|---|
| time | water (incl. | ACN (incl. | [mL/ |
| (min) | 0.04% TFA) | 0.02% TFA) | min] |
| 0.00 | 95 | 5 | 1.5 |
| 0.70 | 5 | 95 | 1.5 |
| 1.16 | 5 | 95 | 1.5 |
| 1.50 | 95 | 5 | 1.5 |

Analytical column: Kinetex EVO C18_2.1×30 mm_5 μm; column temperature: 40° C.

### Method B

| | Vol % | Vol | Flow |
|---|---|---|---|
| time | water (incl. | % | [mL/ |
| (min) | 0.1% NH₃) | ACN | min] |
| 0.00 | 95 | 5 | 1.3 |
| 0.02 | 95 | 5 | 1.3 |
| 1.00 | 0 | 100 | 1.3 |
| 1.30 | 0 | 100 | 1.3 |

Device description: Waters Acquity; Analytical column: XBridge (Waters) BEH C18_2.1×30 mm_2.5 μm; column temperature: 60° C.

### Method C

| | Vol % | Vol | Flow |
|---|---|---|---|
| time | water (incl. | % | [mL/ |
| (min) | 0.1% TFA) | ACN | min] |
| 0.00 | 99 | 1 | 1.6 |
| 0.02 | 99 | 1 | 1.6 |
| 1.00 | 0 | 100 | 1.6 |
| 1.10 | 0 | 100 | 1.6 |

Device description: Waters Acquity; Analytical column: Xbridge (Waters) BEH C18_2.1×30 mm_1.7 μm; column temperature: 60° C.

### Method D

| | Vol. % | Vol. | Flow |
|---|---|---|---|
| time | water (incl. | % | [mL/ |
| (min) | 0.1% NH₄OH) | ACN | min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Sunfire C18_3.0×30 mm-2.5 μm (Waters); column temperature: 60° C.

### Method E

| | Vol % water | | Flow |
|---|---|---|---|
| time (min) | (incl. 0.1% NH₃) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Xbridge (Waters) C18_3.0×30 mm_2.5 μm; column temperature: 60° C.

### Method F

| | Vol % water | Vol % ACN | Flow |
|---|---|---|---|
| time (min) | (incl. 0.04% TFA) | (incl. 0.02% TFA) | [mL/min] |
| 0.01 | 90 | 10 | 1.5 |
| 0.80 | 0 | 100 | 1.5 |
| 1.30 | 0 | 100 | 1.5 |

Device description: Shimadzu LC-20 ADXR; Analytical column: Halo C18_3.0×30 mm_5 μm; column temperature: 40° C.

### Method G

| | Vol % water | | Flow |
|---|---|---|---|
| time (min) | (incl. 0.1% TFA) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Zorbax (Agilent) StableBond C18_3.0×30 mm-1.8 μm; column temperature: 60° C.

### Method H

| | Vol. % water | Vol % ACN | Flow |
|---|---|---|---|
| time (min) | (incl. 0.1% TFA) | (incl. 0.1% FA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: Sunfire (Waters) C18_3.0×30 mm_2.5 μm; column temperature: 60° C.

### Method I

| | Vol. % water | | Flow |
|---|---|---|---|
| time (min) | (incl. 0.1% NH₃) | Vol % MeOH | [mL/min] |
| 0.00 | 95 | 5 | 1.3 |
| 0.02 | 95 | 5 | 1.3 |
| 1.1 | 0 | 100 | 1.3 |
| 1.3 | 0 | 100 | 1.3 |

Device description: Waters Acquity; Analytical column: XBridge BEH (Waters) C18_2.1×30 mm_1.7 μm; column temperature: 60° C.

### Method J

| | Vol. % water | | Flow |
|---|---|---|---|
| time (min) | (incl. 0.1% NH₃) | Vol. % ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: Xbridge (Waters) C18_3.0×30 mm_2.5 μm; column temperature: 60° C.

### Method K

| | Vol. % water | Vol. % CAN | Flow |
|---|---|---|---|
| time (min) | (incl. 0.1% TFA) | (incl. 0.08% TFA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device Description: Waters Acquity, Analytical column: Sunfire C18_3.0×30 mm_2.5 μm (Waters); column temperature: 60° C.

### Method L

| | Vol. % water | | Flow |
|---|---|---|---|
| time (min) | (incl. 0.1% NH₃) | Vol. % ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: XBridge C18_3.0×30 mm_2.5 μm (Waters); column temperature: 60° C.

### Method M

| | Vol % water | Vol % ACN (incl. | Flow |
|---|---|---|---|
| time (min) | (incl. 0.1% TFA) | 0.08% TFA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: Sunfire (Waters) C18_3.0×30 mm_2.5 μm; column temperature: 60° C.

### Method N

| | | iPrOH | Flow |
|---|---|---|---|
| time (min) | Vol. % scCO₂ | (20 mM NH₃) | [mL/min] |
| 0.00 | 70 | 30 | 4.0 |
| 10 | 70 | 30 | 4.0 |

Device description: Agilent 1260 SFC; Column: Chiral ART® Amylose SA, 4.6×250 mm, 2.5 μm (YMC); column temperature: 40° C.

### Method 0

| | Vol. % water | | Flow |
|---|---|---|---|
| time (min) | (incl. 0.1% NH₃) | Vol. % ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: XBridge (Waters) C18_3.0×30 mm_2.5 μm; column temperature: 60° C.

### Method P

| | Vol. % water | Vol. % CAN | Flow |
|---|---|---|---|
| time (min) | (incl. 0.1% TFA) | (incl. 0.08% TFA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: Sunfire C18_3.0×30 mm-2.5 μm (Waters); column temperature: 60° C.

### Method Q

| | Vol % water | | Flow |
|---|---|---|---|
| time (min) | (incl. 0.1% TFA) | Vol % ACN | [mL/min] |
| 0.00 | 95 | 5 | 1.5 |
| 1.30 | 0 | 100 | 1.5 |
| 1.50 | 0 | 100 | 1.5 |

Device description: Waters Acquity; Analytical column: Sunfire (Waters) C18_3.0×30 mm_2.5 μm; column temperature: 60° C.

### Method

| | Vol % water | Vol % CAN | Flow |
|---|---|---|---|
| time (min) | (incl. 0.04% TFA) | (incl. 0.02% TFA) | [mL/min] |
| 0.01 | 90 | 10 | 1.5 |
| 0.80 | 0 | 100 | 1.5 |
| 1.30 | 0 | 100 | 1.5 |

Device description: Shimadzu LC-20ADXR; Analytical column: Halo C18_3.0×30 mm_5 μm; column temperature: 40° C.

### Method S

| | Vol % water | Vol % CAN | Flow |
|---|---|---|---|
| time (min) | (incl. 0.04% TFA) | (incl. 0.02% TFA) | [mL/min] |
| 0.01 | 95 | 5 | 1.5 |
| 0.70 | 5 | 95 | 1.5 |
| 1.16 | 5 | 95 | 1.5 |
| 1.50 | 95 | 5 | |
| 1.51 | 95 | 5 | |

Device description: Agilent 1200 HPLC; Analytical column: Halo C18_3.0×30 mm_5 μm; column temperature: 40° C.

1. A compound of formula (I) 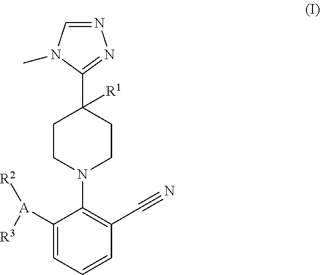
wherein
A is A1a which is a 5- or 6-membered mono-heteroaryl ring containing one or two heteroatom members selected from the group consisting of nitrogen, oxygen and sulphur;
or A is A1b which is a 9- or 10-membered fused bicyclic-heteroaryl ring containing one to four heteroatom members selected from the group consisting of nitrogen, oxygen and sulphur,
wherein at least one of the heteroatom members is nitrogen;
or A is selected from the group A1c consisting of 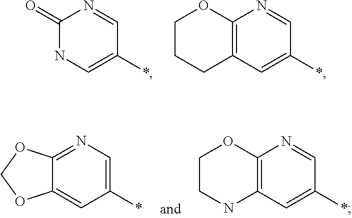
R¹ is selected from the group R1a, consisting of H, C₁₋₄-alkyl and halo;
R² is selected from the group R2a, consisting of H, halo, hydroxy, C₁₋₆-alkyl, C₂₋₆-alkynyl, C₃₋₆-cycloalkyl, F₁₋₉-fluoro-C₁₋₄-alkyl, HO—C₁₋₆-alkyl, C₁₋₆-alkyloxy, C₁₋₄-alkyl-O—H₂CH₂C—O—, C₃₋₆-cycloalkyloxy, C₃₋₆-cycloalkyl-H₂C—O—, F₁₋₉-fluoro-C₁₋₄-alkyloxy, C₁₋₆-alkyl-O—C(O)—, H₂N—C(O)—, and C₁₋₆-alkyl-NH—C(O)—;
or R² is selected from the group R2b, consisting of phenyl, benzyl, phenoxy and benzyloxy, wherein R2b is substituted with one or two R⁴;
or R² is R2c, which is a 5- or 6-membered mono-heteroaryl ring containing one or two heteroatom members selected from the group consisting of nitrogen, oxygen and sulphur,
wherein R2c is substituted with one or two R⁴;
or R² is R2d, consisting of 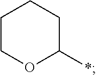
R³ is selected from the group R3a, consisting of H, C₁₋₄-alkyl and halo;
R⁴ is selected from the group R4a, consisting of H, C₁₋₄-alkyl and halo;
or a salt thereof, particularly a pharmaceutically acceptable salt thereof.

2. The compound of formula (I) according to claim 1, wherein A is selected from the group A4 consisting of pyrazolyl, pyridinyl, pyrimidinyl, pyridazinyl, pyrazinyl, methyl-pyrimidonyl, thiazolyl, isothiazolyl, oxazolyl, isoxazolyl, 1,2-dihydropyrimidin-2-onyl, 3H-imidazo[4,5-b]pyridinyl, imidazo[1,2-a]pyrimidinyl, 2H-pyrazolo[3,4-b]pyridinyl, 1H[1,2,3]triazolo[4,5-b]pyridinyl, [1,2,4]triazolo[4,3-a]pyrimidinyl, 1H-pyrazolo[4,3-c]pyridinyl, [1,2,5]oxadiazolo[3,4-b]pyridinyl, [1,2,4]triazolo[1,5-a]pyrimidinyl, [1,2,5]thiadiazolo[3,4-b]pyridinyl, 2H-[1,3]dioxolo[4,5-b]pyridinyl, imidazo[1,2-a]pyrimidinyl, pyrazolo[1,5-b]pyridazinyl, 2H,3H,4H-pyrano[2,3-b]pyridinyl, 1H,2H,3H-pyrido[2,3-b][1,4]oxazinyl and 1,8-naphthyridinyl;
or a salt thereof.

3. The compound of formula (I) according to claim 1, wherein A is selected from the group A7 consisting of 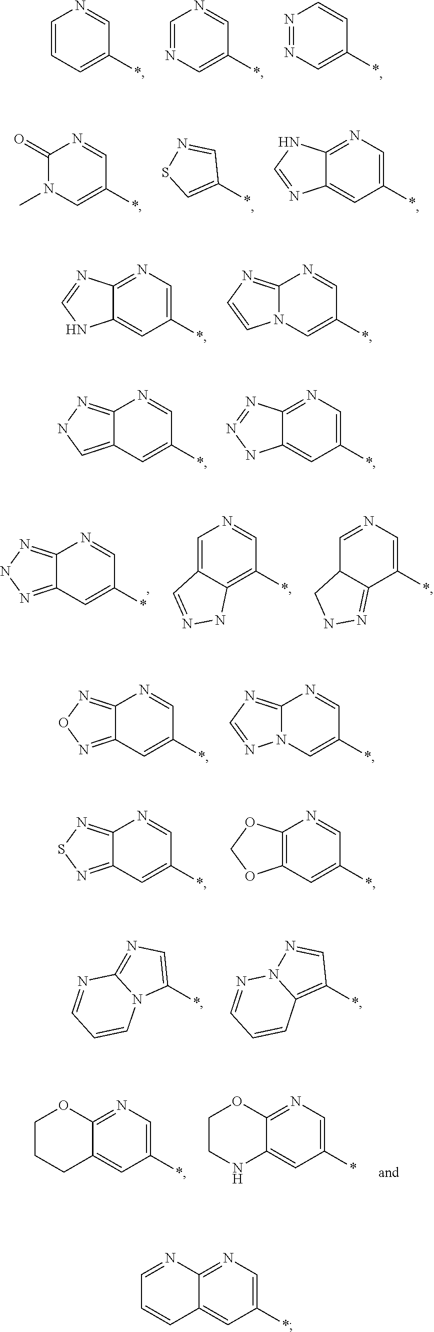
or a salt thereof.

4. The compound of formula (I) according to claim 1, wherein R¹ is selected from the group R1d, consisting of H, H₃C— and F;
or a salt thereof.

5. The compound of formula (I) according to claim 1, wherein R2 is R2E selected from the group R2i, consisting of H, F, Cl, hydroxy, methyl, t-butyl, H₃C-alkynyl, cyclopropyl, F₃C—, F₃CCH₂—, F₂CHCH₂—, F₃C—C(CH₃)₂—, HO—CH₂—, H₃C—O—, (H₃C)₂CH—O—, H₃C—O—H₂CH₂C—O—, F₂HC—O—, F₃C—O—, H₃C—O—C(O)—, H₂N—C(O)—, H₃C—NH—C(O)—, 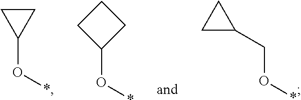
or R2E is selected from the group R2j, consisting of phenyl, m-chlorophenyl, benzyl, phenoxy and benzyloxy;
or R2E is R2g, which is selected from the group consisting of 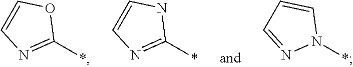
wherein R2g is substituted with H or methyl;
or R2E is R2d, consisting of 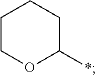
or a salt thereof.

6. The compound of formula (I) according to claim 1, wherein R³ is selected from the group R3e, consisting of F; or a salt thereof.

7. The compound of formula (I) according to claim 1, having formula (1-ϵ) 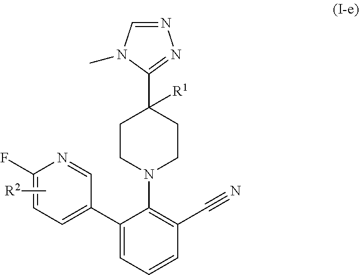
or a salt thereof.

8. The compound of formula (I) according to claim 1 having formula (1-a) 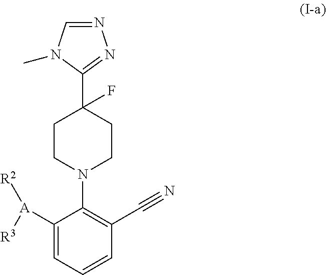
or a salt thereof.

9. The compound of formula (I) according to claim 1, selected from the group consisting of 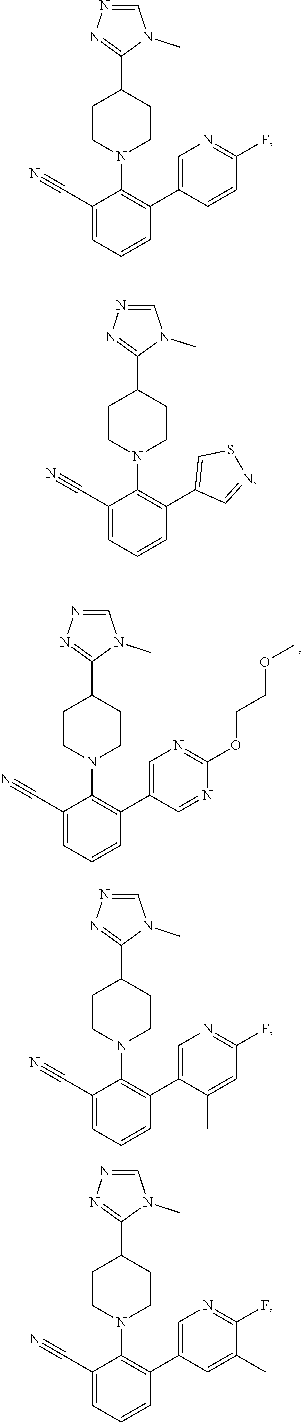 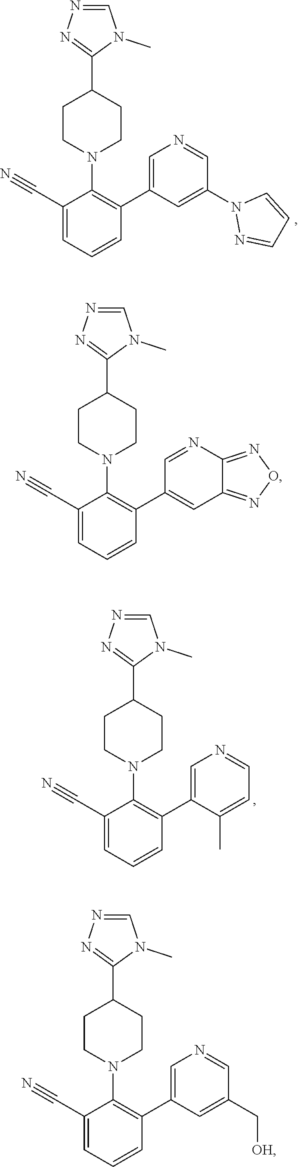 [SMILES: Cn1cnnc1C1CCN(c2c(C#N)cccc2-c2cncc(-n3cccn3)c2)CC1 ; Cn1cnnc1C1CCN(c2c(C#N)cccc2-c2cnc3nonc3c2)CC1 ; Cc1ccncc1-c1cccc(C#N)c1N1CCC(c2nncn2C)CC1 ; Cn1cnnc1C1CCN(c2c(C#N)cccc2-c2cncc(CO)c2)CC1] 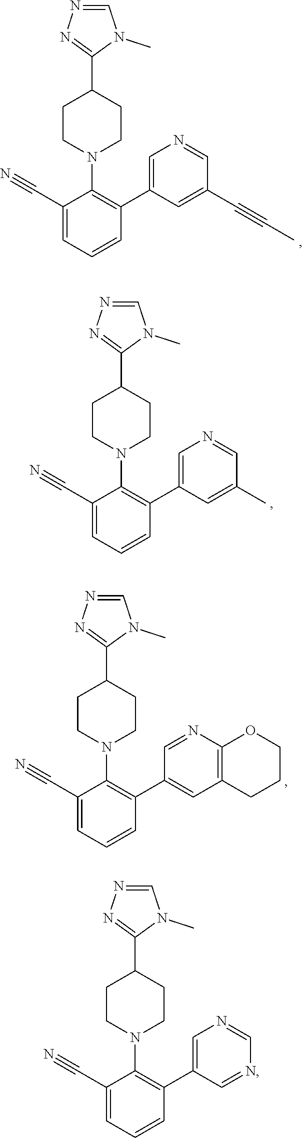 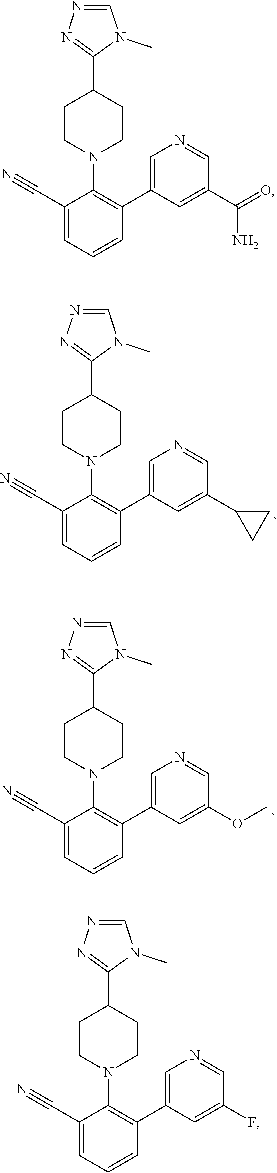 [SMILES: Cn1cnnc1C1CCN(c2c(C#N)cccc2-c2cncc(C(N)=O)c2)CC1 ; Cn1cnnc1C1CCN(c2c(C#N)cccc2-c2cncc(C3CC3)c2)CC1 ; COc1cncc(-c2cccc(C#N)c2N2CCC(c3nncn3C)CC2)c1 ; Cn1cnnc1C1CCN(c2c(C#N)cccc2-c2cncc(F)c2)CC1] 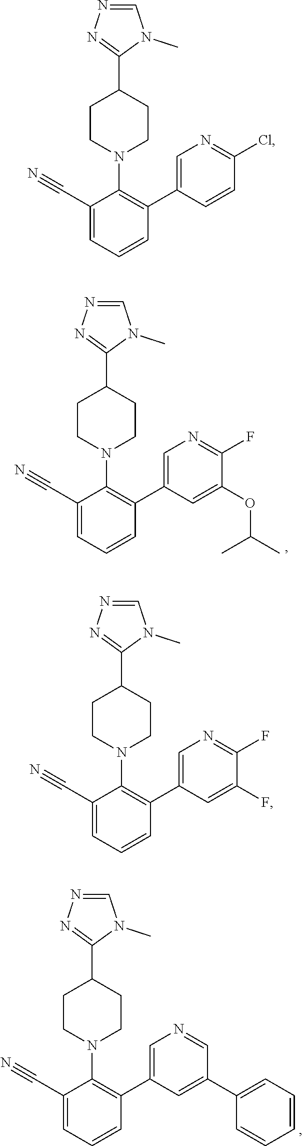 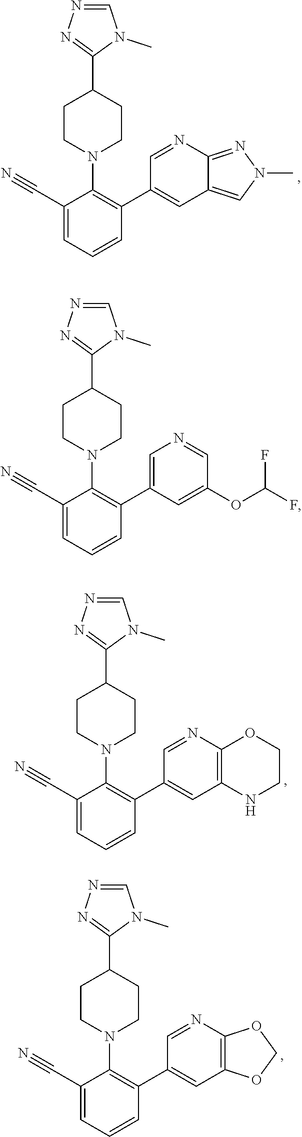 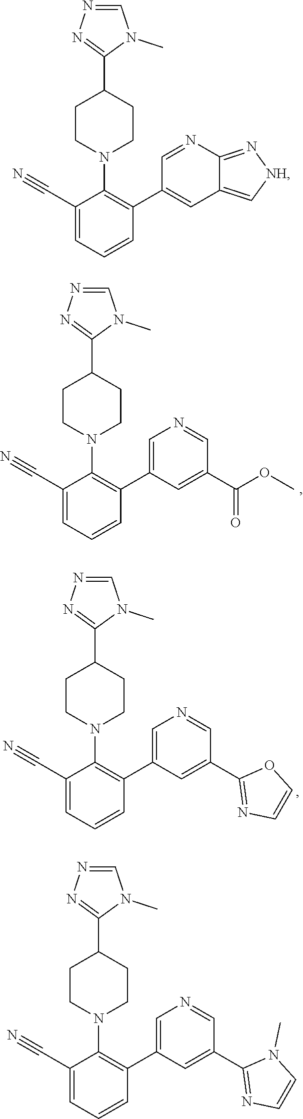 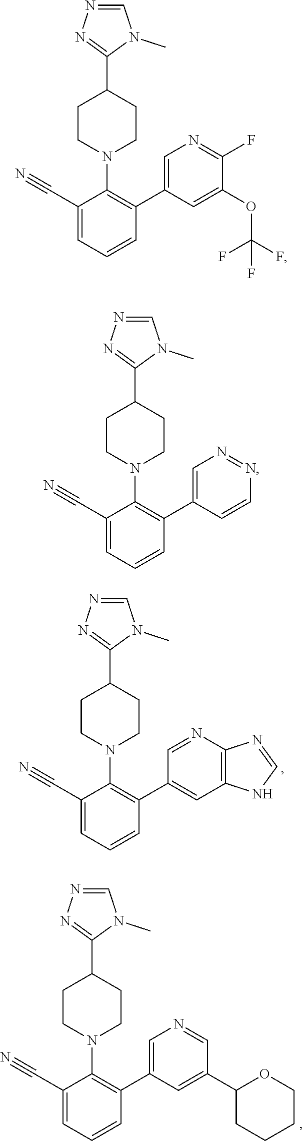 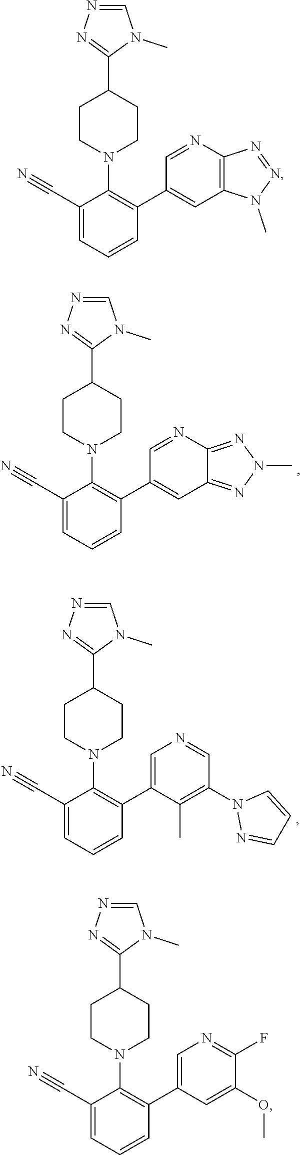 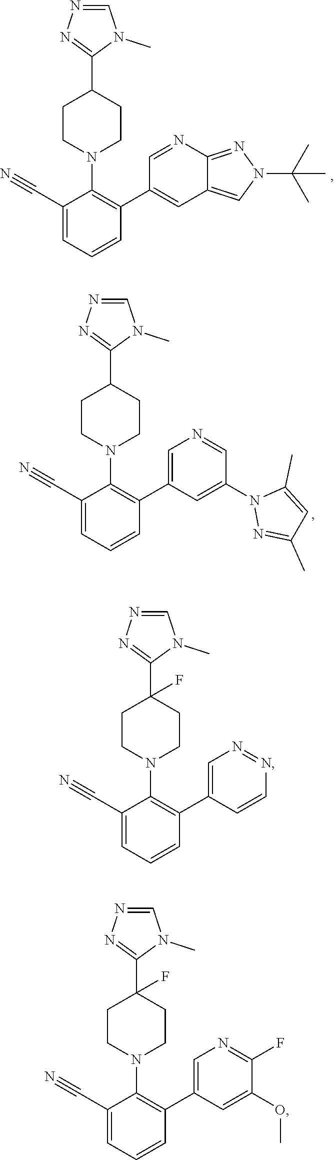 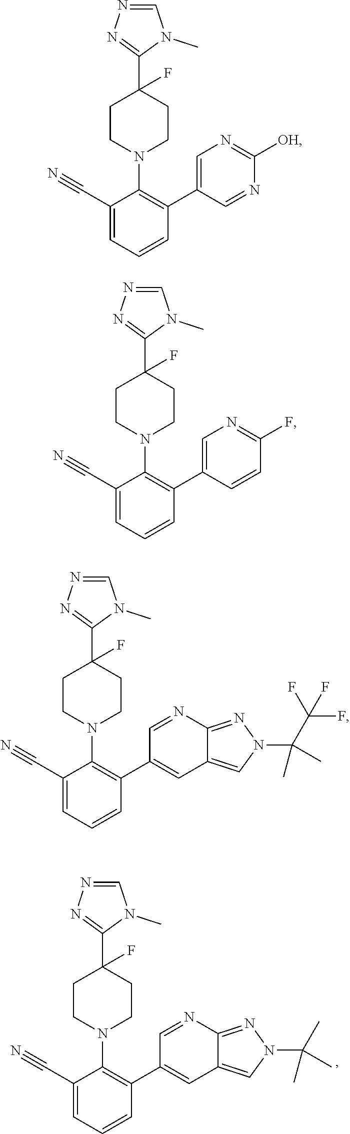 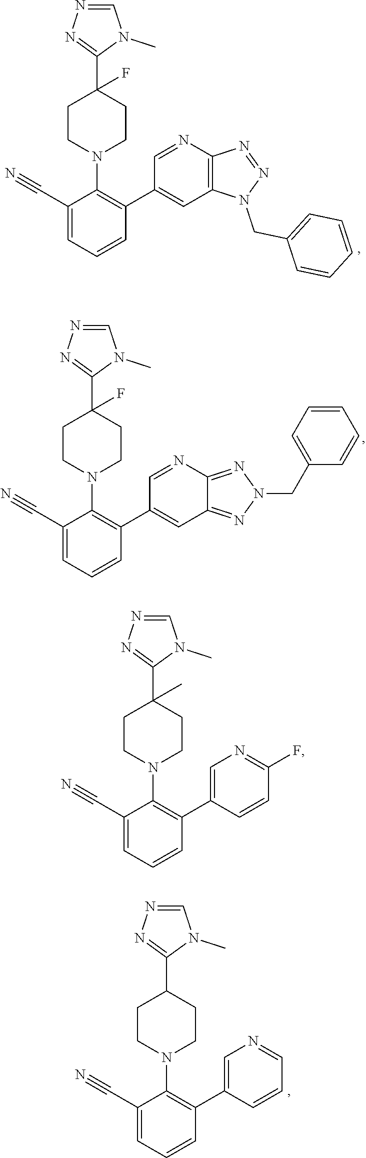 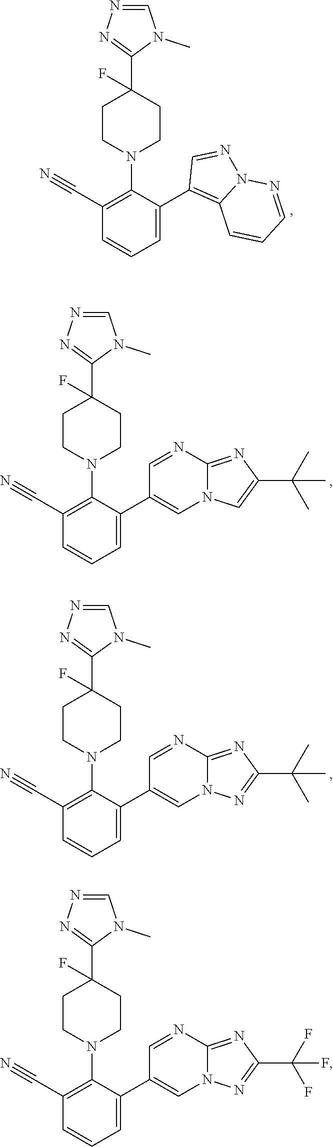 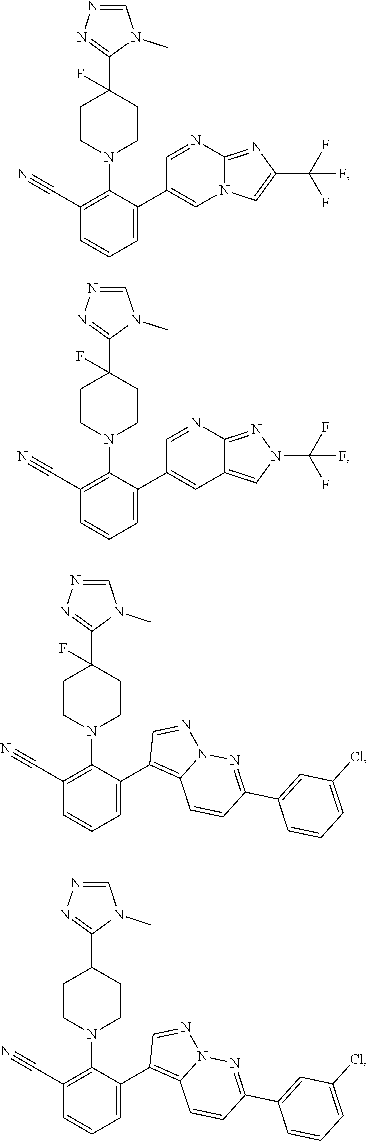 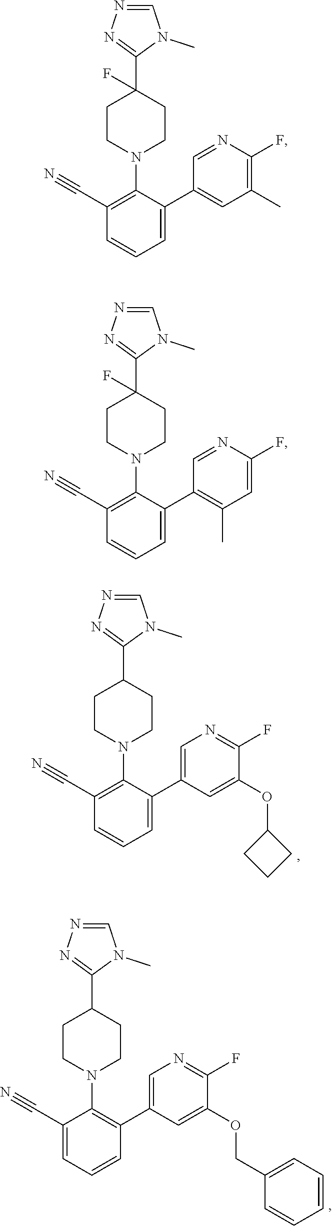 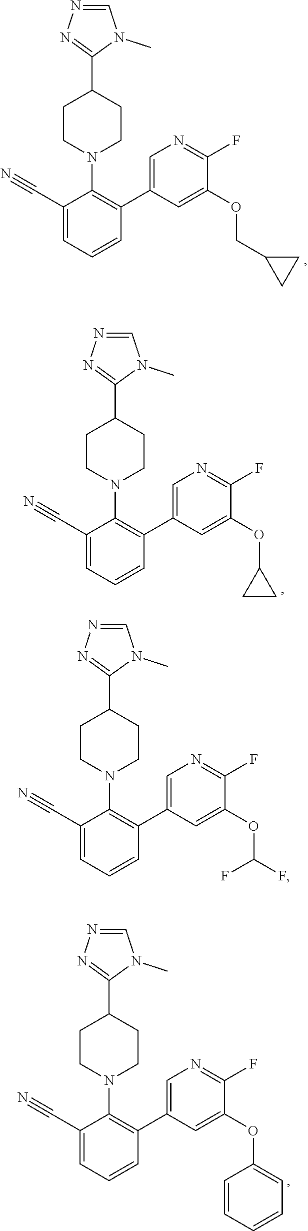 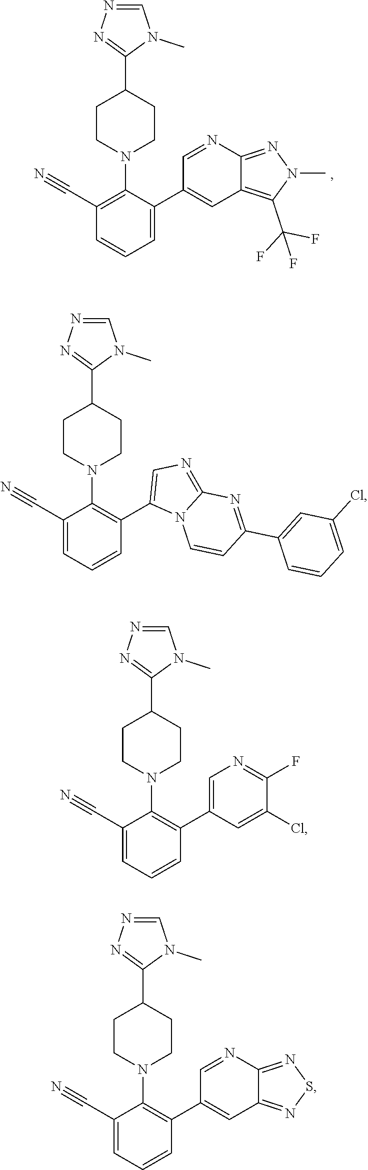 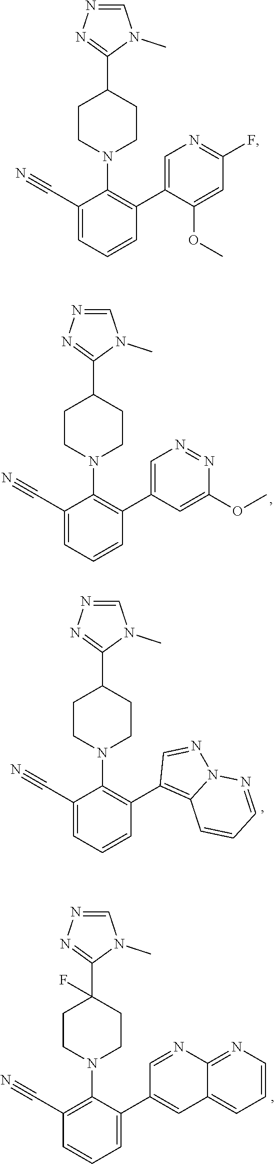 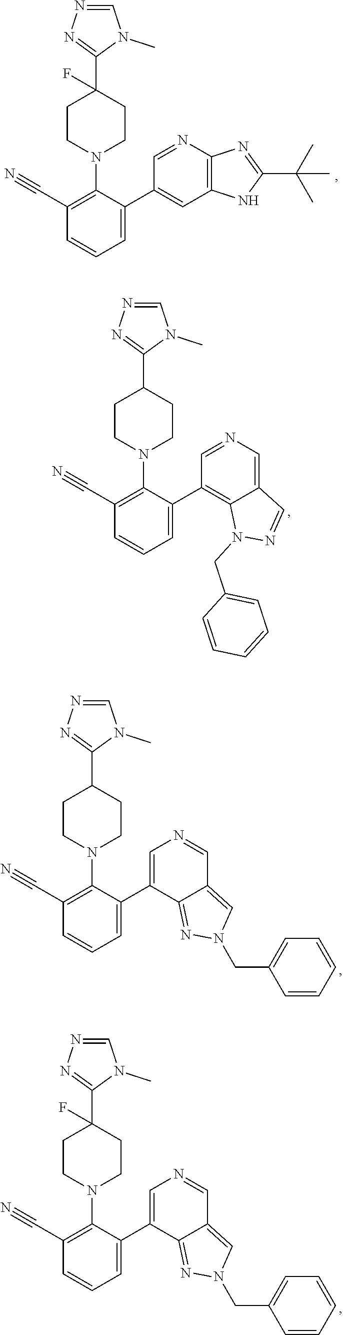 [SMILES: Cn1cnnc1C1(F)CCN(c2c(C#N)cccc2-c2cnc3nc(C(C)(C)C)[nH]c3c2)CC1 ; Cn1cnnc1C1CCN(c2c(C#N)cccc2-c2cncc3cnn(Cc4ccccc4)c23)CC1 ; Cn1cnnc1C1CCN(c2c(C#N)cccc2-c2cncc3cn(Cc4ccccc4)nc23)CC1 ; Cn1cnnc1C1(F)CCN(c2c(C#N)cccc2-c2cncc3cn(Cc4ccccc4)nc23)CC1] 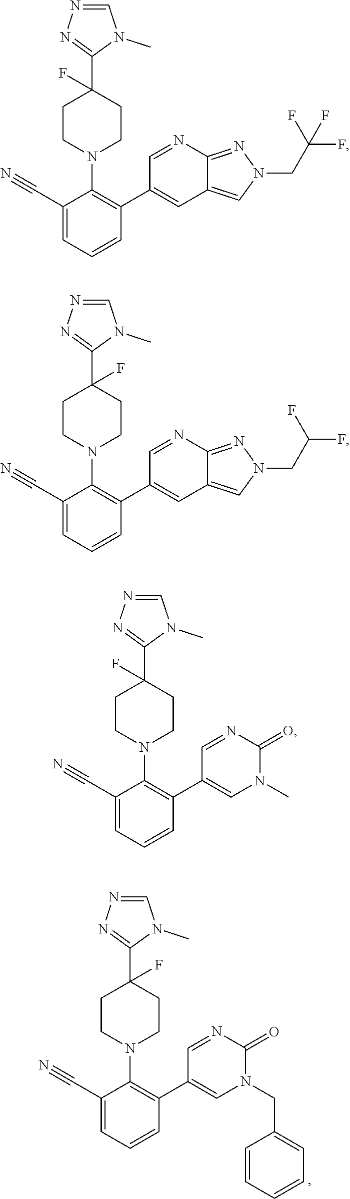 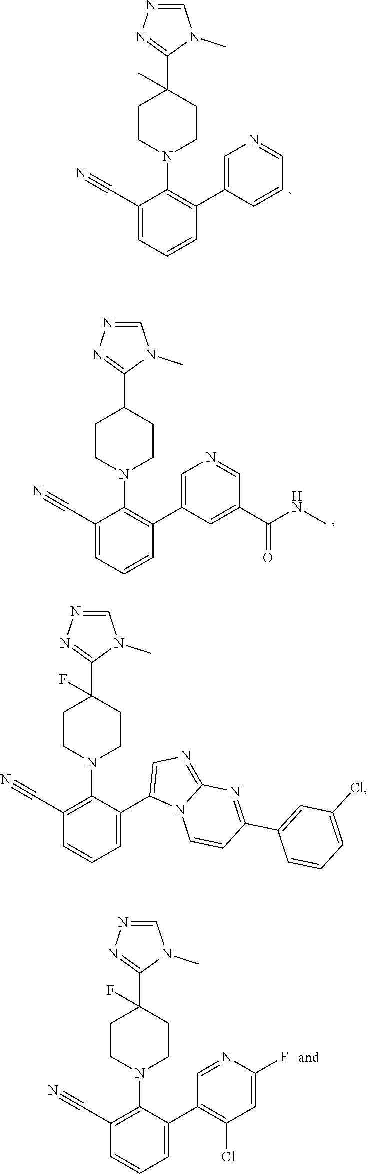 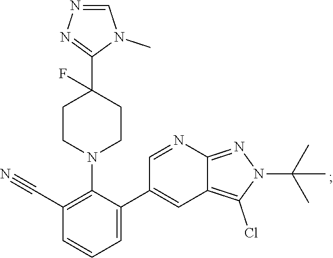 [SMILES: Cn1cnnc1C1(F)CCN(c2c(C#N)cccc2-c2cnc3nn(C(C)(C)C)c(Cl)c3c2)CC1]
or a salt thereof.

10. A pharmaceutically acceptable salt of a compound according to claim 1.

11. A pharmaceutical composition comprising one or more compounds according to claim 1, or pharmaceutically acceptable salts thereof, together with one or more inert carriers and/or diluents.

12. A pharmaceutical composition comprising one or more compounds according to claim 1, or pharmaceutically acceptable salts thereof, and one or more additional therapeutic agents, together with one or more inert carriers and/or diluents.

13. The pharmaceutical composition according to claim 12 wherein the one or more additional therapeutic agents are selected from the group consisting of anticancer agents and antifibrotic agents.

14. (canceled)

15. A method for the treatment of one or more diseases, comprising administering a therapeutically effective amount of a compound of claim 1 to a patient in need thereof, wherein the disease is cancer, a fibrotic diseases, a neurodegenerative disease, atherosclerosis, an infectious disease, or a chronic kidney disease.

16. (canceled)
